SEQUENCE LISTING (311 sequences)

SEQ ID NO: 1 - protein, 28 aa, synthetic construct
  REGION 1..28: note = synthetic peptide
ADDQNPWRAYLDLLFPTDTLLLDLLWCG

SEQ ID NO: 2 - protein, 35 aa, synthetic construct
  REGION 1..35: note = synthetic peptide
AEQNPIYWARYADWLFTTPLLLLDLALLVDADECG

SEQ ID NO: 3 - protein, 32 aa, synthetic construct
  REGION 1..32: note = synthetic peptide
ADDQNPWRAYLDLLFPTDTLLLDLLWDADECG

SEQ ID NO: 4 - protein, 39 aa, synthetic construct
  REGION 1..39: note = synthetic peptide
  SITE 1: note = MISC_FEATURE - acetylated
AAEQNPIYWARYADWLFTTPLLLLDLALLVDADEGTKCG

SEQ ID NO: 5 - protein, 37 aa, synthetic construct
  REGION 1..37: note = synthetic peptide
AAEQNPIYWARYADWLFTTPLLLLDLALLVDADEGTC

SEQ ID NO: 6 - protein, 39 aa, unidentified
  REGION 1..39: note = Description of Unknown: pH-sensitive membrane polypeptide
AAEQNPIYWWARYADWLFTTPLLLLDLALLVDADEGTCG

SEQ ID NO: 7 - protein, 35 aa, unidentified
  REGION 1..35: note = Description of Unknown: Wild-type pH-sensitive membrane polypeptide
AEQNPIYWARYADWLFTTPLLLLDLALLVDADEGT

SEQ ID NO: 8 - protein, 36 aa, unidentified
  REGION 1..36: note = Description of Unknown: Wild-type pH-sensitive membrane polypeptide
GGEQNPIYWARYADWLFTTPLLLLDLALLVDADEGT

SEQ ID NO: 9 - protein, 35 aa, unidentified; identical to SEQ ID NO: 7
  REGION 1..35: note = Description of Unknown: Wild-type pH-sensitive membrane polypeptide

SEQ ID NO: 10 - protein, 38 aa, synthetic construct
  REGION 1..38: note = Description of Artificial Sequence: Synthetic polypeptide
AAEQNPIYWARYADWLFTTPLLLLDLALLVDADEGTCG

SEQ ID NO: 11 - protein, 38 aa, synthetic construct
  REGION 1..38: note = Description of Artificial Sequence: Synthetic polypeptide
GGEQNPIYWARYADWLFTTPLLLLDLALLVDADEGTCG

SEQ ID NO: 12 - protein, 37 aa, synthetic construct
  REGION 1..37: note = Description of Artificial Sequence: Synthetic polypeptide
ACEQNPIYWARYADWLFTTPLLLLDLALLVDADEGTG

SEQ ID NO: 13 - protein, 36 aa, synthetic construct
  REGION 1..36: note = Description of Artificial Sequence: Synthetic polypeptide
ACEQNPIYWARYADWLFTTPLLLLDLALLVDADEGT

SEQ ID NO: 14 - protein, 36 aa, synthetic construct
  REGION 1..36: note = Description of Artificial Sequence: Synthetic polypeptide
AKEQNPIYWARYADWLFTTPLLLLDLALLVDADEGT

SEQ ID NO: 15 - protein, 39 aa, synthetic construct; identical to SEQ ID NO: 4
  REGION 1..39: note = Description of Artificial Sequence: Synthetic polypeptide

SEQ ID NO: 16 - protein, 36 aa, synthetic construct
  REGION 1..36: note = Description of Artificial Sequence: Synthetic polypeptide
AKEQNPIYWARYADWLFTTPLLLLDLALLVDADECT

SEQ ID NO: 17 - protein, 37 aa, synthetic construct
  REGION 1..37: note = Description of Artificial Sequence: Synthetic polypeptide
ACEQNPIYWARYANWLFTTPLLLLNLALLVDADEGTG

SEQ ID NO: 18 - protein, 37 aa, synthetic construct
  REGION 1..37: note = Description of Artificial Sequence: Synthetic polypeptide
ACEQNPIYWARYAKWLFTTPLLLLKLALLVDADEGTG

SEQ ID NO: 19 - protein, 36 aa, synthetic construct
  REGION 1..36: note = Description of Artificial Sequence: Synthetic polypeptide
GGEQNPIYWARYADWLFTTPLLLLDLALLVNANQGT

SEQ ID NO: 20 - protein, 36 aa, synthetic construct
  REGION 1..36: note = Description of Artificial Sequence: Synthetic polypeptide
AAEQNPIYWARYADWLFTTPLLLLALALLVDADEGT

SEQ ID NO: 21 - protein, 36 aa, synthetic construct
  REGION 1..36: note = Description of Artificial Sequence: Synthetic polypeptide
AAEQNPIYWARYAAWLFTTPLLLLDLALLVDADEGT

SEQ ID NO: 22 - protein, 36 aa, synthetic construct
  REGION 1..36: note = Description of Artificial Sequence: Synthetic polypeptide
AAEQNPIYWARYADWLFTTALLLLDLALLVDADEGT

SEQ ID NO: 23 - protein, 36 aa, synthetic construct
  REGION 1..36: note = Description of Artificial Sequence: Synthetic polypeptide
AAEQNPIYWARYADWLFTTPLLLLELALLVDADEGT

SEQ ID NO: 24 - protein, 36 aa, synthetic construct
  REGION 1..36: note = Description of Artificial Sequence: Synthetic polypeptide
AAEQNPIYWARYAEWLFTTPLLLLDLALLVDADEGT

SEQ ID NO: 25 - protein, 39 aa, synthetic construct
  REGION 1..39: note = Description of Artificial Sequence: Synthetic polypeptide
AAEQNPIIYWARYADWLFTDLPLLLLDLLALLVDADEGT

SEQ ID NO: 26 - protein, 37 aa, synthetic construct
  REGION 1..37: note = Description of Artificial Sequence: Synthetic polypeptide
GEQNPIYWAQYADWLFTTPLLLLDLALLVDADEGTCG

SEQ ID NO: 27 - protein, 38 aa, synthetic construct
  REGION 1..38: note = Description of Artificial Sequence: Synthetic polypeptide
GGEQNPIYWARYADWLFTTPLLLDLLALLVDADEGTCG

SEQ ID NO: 28 - protein, 38 aa, synthetic construct
  REGION 1..38: note = Description of Artificial Sequence: Synthetic polypeptide
GGEQNPIYWARYADWLFTTPLLLLLDALLVDADEGTCG

SEQ ID NO: 29 - protein, 38 aa, synthetic construct
  REGION 1..38: note = Description of Artificial Sequence: Synthetic polypeptide
GGEQNPIYWARYDAWLFTTPLLLLDLALLVDADEGTCG

SEQ ID NO: 30 - protein, 38 aa, synthetic construct
  REGION 1..38: note = Description of Artificial Sequence: Synthetic polypeptide
GGEQNPIYWARYAWDLFTTPLLLLDLALLVDADEGTCG

SEQ ID NO: 31 - protein, 36 aa, synthetic construct
  REGION 1..36: note = Description of Artificial Sequence: Synthetic polypeptide
AAEQNPIYWARYADWLFTTGLLLLDLALLVDADEGT

SEQ ID NO: 32 - protein, 37 aa, synthetic construct
  REGION 1..37: note = Description of Artificial Sequence: Synthetic polypeptide
DDDEDNPIYWARYADWLFTTPLLLLHGALLVDADECT

SEQ ID NO: 33 - protein, 38 aa, synthetic construct
  REGION 1..38: note = Description of Artificial Sequence: Synthetic polypeptide
DDDEDNPIYWARYAHWLFTTPLLLLHGALLVDADEGCT

SEQ ID NO: 34 - protein, 37 aa, synthetic construct
  REGION 1..37: note = Description of Artificial Sequence: Synthetic polypeptide
DDDEDNPIYWARYAHWLFTTPLLLLHGALLVNADECT

SEQ ID NO: 35 - protein, 37 aa, synthetic construct
  REGION 1..37: note = Description of Artificial Sequence: Synthetic polypeptide
DDDEDNPIYWARYAHWLFTTPLLLLHGALLVNANECT

SEQ ID NO: 36 - protein, 34 aa, synthetic construct
  REGION 1..34: note = Description of Artificial Sequence: Synthetic polypeptide
AEQNPIYWARYADFLFTTPLLLLDLALLVDADET

SEQ ID NO: 37 - protein, 35 aa, synthetic construct
  REGION 1..35: note = Description of Artificial Sequence: Synthetic polypeptide
AEQNPIYFARYADWLFTTPLLLLDLALLVDADEGT

SEQ ID NO: 38 - protein, 34 aa, synthetic construct
  REGION 1..34: note = Description of Artificial Sequence: Synthetic polypeptide
AEQNPIYFARYADFLFTTPLLLLDLALLWDADET

SEQ ID NO: 39 - protein, 32 aa, synthetic construct
  REGION 1..32: note = Description of Artificial Sequence: Synthetic polypeptide
AKEDQNPYWARYADWLFTTPLLLLDLALLVDG

SEQ ID NO: 40 - protein, 32 aa, synthetic construct
  REGION 1..32: note = Description of Artificial Sequence: Synthetic polypeptide
ACEDQNPYWARYADWLFTTPLLLLDLALLVDG

SEQ ID NO: 41 - protein, 32 aa, synthetic construct
  REGION 1..32: note = Description of Artificial Sequence: Synthetic polypeptide
AEDQNPYWARYADWLFTTPLLLLDLALLVDCG

SEQ ID NO: 42 - protein, 32 aa, synthetic construct
  REGION 1..32: note = Description of Artificial Sequence: Synthetic polypeptide
AEDQNPYWARYADWLFTTPLLLLELALLVECG

SEQ ID NO: 43 - protein, 30 aa, synthetic construct
  REGION 1..30: note = Description of Artificial Sequence: Synthetic polypeptide
AKEDQNPYWRAYADLFTPLTLLDLLALWDG

SEQ ID NO: 44 - protein, 30 aa, synthetic construct
  REGION 1..30: note = Description of Artificial Sequence: Synthetic polypeptide
ACEDQNPYWRAYADLFTPLTLLDLLALWDG

SEQ ID NO: 45 - protein, 27 aa, synthetic construct
  REGION 1..27: note = Description of Artificial Sequence: Synthetic peptide
ACDDQNPWRAYLDLLFPTDTLLLDLLW

SEQ ID NO: 46 - protein, 34 aa, synthetic construct
  REGION 1..34: note = Description of Artificial Sequence: Synthetic polypeptide
TEDADVLLALDLLLLPTTFLWDAYRAWYPNQECA

SEQ ID NO: 47 - protein, 20 aa, synthetic construct
  REGION 1..20: note = Description of Artificial Sequence: Synthetic peptide
AEQNPIYWARYADWLFTTPL

SEQ ID NO: 48 - protein, 21 aa, synthetic construct
  REGION 1..21: note = Description of Artificial Sequence: Synthetic peptide
AEQNPIYWARYADWLFTTPCL

SEQ ID NO: 49 - protein, 21 aa, synthetic construct
  REGION 1..21: note = Description of Artificial Sequence: Synthetic peptide
ACEQNPIYWARYADWLFTTPL

SEQ ID NO: 50 - protein, 20 aa, synthetic construct
  REGION 1..20: note = Description of Artificial Sequence: Synthetic peptide
AEQNPIYFARYADWLFTTPL

SEQ ID NO: 51 - protein, 21 aa, synthetic construct
  REGION 1..21: note = Description of Artificial Sequence: Synthetic peptide
KEDQNPWARYADLLFPTTLAW

SEQ ID NO: 52 - protein, 22 aa, synthetic construct
  REGION 1..22: note = Description of Artificial Sequence: Synthetic peptide
ACEDQNPWARYADLLFPTTLAW

SEQ ID NO: 53 - protein, 24 aa, synthetic construct
  REGION 1..24: note = Description of Artificial Sequence: Synthetic peptide
ACEDQNPWARYADWLFPTTLLLLD

SEQ ID NO: 54 - protein, 22 aa, synthetic construct
  REGION 1..22: note = Description of Artificial Sequence: Synthetic peptide
ACEEQNPWARYAELLFPTTLAW

SEQ ID NO: 55 - protein, 24 aa, synthetic construct
  REGION 1..24: note = Description of Artificial Sequence: Synthetic peptide
ACEEQNPWARYAEWLFPTTLLLLE

SEQ ID NO: 56 - protein, 25 aa, synthetic construct
  REGION 1..25: note = Description of Artificial Sequence: Synthetic peptide
ACEEQNPWARYLEWLFPTETLLLEL

SEQ ID NO: 57 - protein, 36 aa, synthetic construct; identical to SEQ ID NO: 8
  REGION 1..36: note = Description of Artificial Sequence: Synthetic polypeptide

SEQ ID NO: 58 - protein, 35 aa, synthetic construct
  REGION 1..35: note = Description of Artificial Sequence: Synthetic polypeptide
ACEQNPIYWARYADWLFTTPLLLLDLALLVDADET

SEQ ID NO: 59 - protein, 38 aa, synthetic construct; identical to SEQ ID NO: 10
  REGION 1..38: note = Description of Artificial Sequence: Synthetic polypeptide

SEQ ID NO: 60 - protein, 36 aa, synthetic construct
  REGION 1..36: note = Description of Artificial Sequence: Synthetic polypeptide
AEQNPIYWARYADWLFTTPLLLLDLALLVDADEGCT

SEQ ID NO: 61 - protein, 38 aa, synthetic construct; identical to SEQ ID NO: 11
  REGION 1..38: note = Description of Artificial Sequence: Synthetic polypeptide

SEQ ID NO: 62 - protein, 36 aa, synthetic construct; identical to SEQ ID NO: 13
  REGION 1..36: note = Description of Artificial Sequence: Synthetic polypeptide

SEQ ID NO: 63 - protein, 36 aa, synthetic construct; identical to SEQ ID NO: 14
  REGION 1..36: note = Description of Artificial Sequence: Synthetic polypeptide

SEQ ID NO: 64 - protein, 36 aa, synthetic construct; identical to SEQ ID NO: 16
  REGION 1..36: note = Description of Artificial Sequence: Synthetic polypeptide

SEQ ID NO: 65 - protein, 36 aa, synthetic construct; identical to SEQ ID NO: 22
  REGION 1..36: note = Description of Artificial Sequence: Synthetic polypeptide

SEQ ID NO: 66 - protein, 37 aa, synthetic construct
  REGION 1..37: note = Description of Artificial Sequence: Synthetic polypeptide
ACAEQNPIYWARYADWLFTTGLLLLDLALLVDADEGT

SEQ ID NO: 67 - protein, 34 aa, synthetic construct; identical to SEQ ID NO: 36
  REGION 1..34: note = Description of Artificial Sequence: Synthetic polypeptide

SEQ ID NO: 68 - protein, 35 aa, synthetic construct; identical to SEQ ID NO: 37
  REGION 1..35: note = Description of Artificial Sequence: Synthetic polypeptide

SEQ ID NO: 69 - protein, 34 aa, synthetic construct; identical to SEQ ID NO: 38
  REGION 1..34: note = Description of Artificial Sequence: Synthetic polypeptide

SEQ ID NO: 70 - protein, 32 aa, synthetic construct; identical to SEQ ID NO: 39
  REGION 1..32: note = Description of Artificial Sequence: Synthetic polypeptide

SEQ ID NO: 71 - protein, 32 aa, synthetic construct; identical to SEQ ID NO: 40
  REGION 1..32: note = Description of Artificial Sequence: Synthetic polypeptide

SEQ ID NO: 72 - protein, 31 aa, synthetic construct
  REGION 1..31: note = Description of Artificial Sequence: Synthetic polypeptide
AEDQNPYWARYADWLFTTPLLLLDLALLVDG

SEQ ID NO: 73 - protein, 32 aa, synthetic construct; identical to SEQ ID NO: 42
  REGION 1..32: note = Description of Artificial Sequence: Synthetic polypeptide

SEQ ID NO: 74 - protein, 30 aa, synthetic construct; identical to SEQ ID NO: 43
  REGION 1..30: note = Description of Artificial Sequence: Synthetic polypeptide

SEQ ID NO: 75 - protein, 30 aa, synthetic construct; identical to SEQ ID NO: 44
  REGION 1..30: note = Description of Artificial Sequence: Synthetic polypeptide

SEQ ID NO: 76 - protein, 32 aa, synthetic construct
  REGION 1..32: note = Description of Artificial Sequence: Synthetic polypeptide
AKEDQNDPYWARYADWLFTTPLLLLDLALLVG

SEQ ID NO: 77 - protein, 34 aa, synthetic construct; identical to SEQ ID NO: 46
  REGION 1..34: note = Description of Artificial Sequence: Synthetic polypeptide

SEQ ID NO: 78 - protein, 36 aa, unidentified; identical to SEQ ID NO: 8
  REGION 1..36: note = Description of Unknown: Wild-type pH-sensitive membrane polypeptide

SEQ ID NO: 79 - protein, 20 aa, synthetic construct
  REGION 1..20: note = Description of Artificial Sequence: Synthetic peptide
AEQNPIYWARYADWLFTTPL

SEQ ID NO: 80 - protein, 21 aa, synthetic construct
  REGION 1..21: note = Description of Artificial Sequence: Synthetic peptide
AEQNPIYWARYADWLFTTPCL

SEQ ID NO: 81 - protein, 21 aa, synthetic construct
  REGION 1..21: note = Description of Artificial Sequence: Synthetic peptide
ACEQNPIYWARYADWLFTTPL

SEQ ID NO: 82 - protein, 21 aa, synthetic construct
  REGION 1..21: note = Description of Artificial Sequence: Synthetic peptide
ACEQNPIYFARYADWLFTTPL

SEQ ID NO: 83 - protein, 27 aa, synthetic construct
  REGION 1..27: note = Description of Artificial Sequence: Synthetic peptide
ACDDQNPWRAYLDLLFPTDTLLLDLLW

SEQ ID NO: 84 - protein, 27 aa, synthetic construct
  REGION 1..27: note = Description of Artificial Sequence: Synthetic peptide
ACEEQNPWRAYLELLFPTETLLLELLW

SEQ ID NO: 85 - protein, 25 aa, synthetic construct
  REGION 1..25: note = Description of Artificial Sequence: Synthetic peptide
ACDDQNPWARYLDWLFPTDTLLLDL

SEQ ID NO: 86 - protein, 24 aa, synthetic construct
  REGION 1..24: note = Description of Artificial Sequence: Synthetic peptide
CDNNNPWRAYLDLLFPTDTLLLDW

SEQ ID NO: 87 - protein, 25 aa, synthetic construct
  REGION 1..25: note = Description of Artificial Sequence: Synthetic peptide
ACEEQNPWARYLEWLFPTETLLLEL

SEQ ID NO: 88 - protein, 24 aa, synthetic construct
  REGION 1..24: note = Description of Artificial Sequence: Synthetic peptide
ACEDQNPWARYADWLFPTTLLLLD

SEQ ID NO: 89 - protein, 24 aa, synthetic construct
  REGION 1..24: note = Description of Artificial Sequence: Synthetic peptide
ACEEQNPWARYAEWLFPTTLLLLE

SEQ ID NO: 90 - protein, 22 aa, synthetic construct
  REGION 1..22: note = Description of Artificial Sequence: Synthetic peptide
ACEDQNPWARYADLLFPTTLAW

SEQ ID NO: 91 - protein, 21 aa, synthetic construct
  REGION 1..21: note = Description of Artificial Sequence: Synthetic peptide
ACEDQNPWARYAELLFPTTLW

SEQ ID NO: 92 - protein, 20 aa, synthetic construct
  REGION 1..20: note = Description of Artificial Sequence: Synthetic peptide
KEDQNPWARYADLLFPTTLW

SEQ ID NO: 93 - protein, 37 aa, synthetic construct
  REGION 1..37: note = Description of Artificial Sequence: Synthetic polypeptide
DDDEDNPIYWARYAHWLFTTPLLLLHGALLVDADECT

SEQ ID NO: 94 - protein, 37 aa, synthetic construct
  REGION 1..37: note = Description of Artificial Sequence: Synthetic polypeptide
DDDEDNPIYWARYAHWLFTTPLLLLDGALLVDADECT

SEQ ID NO: 95 - protein, 37 aa, synthetic construct; identical to SEQ ID NO: 34
  REGION 1..37: note = Description of Artificial Sequence: Synthetic polypeptide

SEQ ID NO: 96 - protein, 37 aa, synthetic construct; identical to SEQ ID NO: 35
  REGION 1..37: note = Description of Artificial Sequence: Synthetic polypeptide

SEQ ID NO: 97 - protein, 37 aa, synthetic construct; identical to SEQ ID NO: 32
  REGION 1..37: note = Description of Artificial Sequence: Synthetic polypeptide

SEQ ID NO: 98 - protein, 37 aa, synthetic construct; identical to SEQ ID NO: 12
  REGION 1..37: note = Description of Artificial Sequence: Synthetic polypeptide

SEQ ID NO: 99 - protein, 35 aa, synthetic construct; identical to SEQ ID NO: 58
  REGION 1..35: note = Description of Artificial Sequence: Synthetic polypeptide

SEQ ID NO: 100 - protein, 36 aa, synthetic construct; identical to SEQ ID NO: 13
  REGION 1..36: note = Description of Artificial Sequence: Synthetic polypeptide

SEQ ID NO: 101 - protein, 38 aa, synthetic construct; identical to SEQ ID NO: 27
  REGION 1..38: note = Description of Artificial Sequence: Synthetic polypeptide

SEQ ID NO: 102 - protein, 38 aa, synthetic construct; identical to SEQ ID NO: 28
  REGION 1..38: note = Description of Artificial Sequence: Synthetic polypeptide

SEQ ID NO: 103 - protein, 38 aa, synthetic construct; identical to SEQ ID NO: 30
  REGION 1..38: note = Description of Artificial Sequence: Synthetic polypeptide

SEQ ID NO: 104 - protein, 38 aa, synthetic construct
  REGION 1..38: note = Description of Artificial Sequence: Synthetic polypeptide
AAEQNPIYWARYAEWLFTTPLLLLDLALLVDADEGTCG

SEQ ID NO: 105 - protein, 38 aa, synthetic construct
  REGION 1..38: note = Description of Artificial Sequence: Synthetic polypeptide
AAEQNPIYWARYAEWLFTTPLLLLELALLVDADEGTCG

SEQ ID NO: 106 - protein, 38 aa, synthetic construct; identical to SEQ ID NO: 29
  REGION 1..38: note = Description of Artificial Sequence: Synthetic polypeptide

SEQ ID NO: 107 - protein, 38 aa, synthetic construct
  REGION 1..38: note = Description of Artificial Sequence: Synthetic polypeptide
GGEQNPIYWAQYDAWLFTTPLLLLDLALLVDADEGTCG

SEQ ID NO: 108 - protein, 38 aa, synthetic construct
  REGION 1..38: note = Description of Artificial Sequence: Synthetic polypeptide
GGEQNPIYWAQDYAWLFTTPLLLLDLALLVDADEGTCG

SEQ ID NO: 109 - protein, 38 aa, synthetic construct
  REGION 1..38: note = Description of Artificial Sequence: Synthetic polypeptide
AAEQNPIYWARYAAWLFTTPLLLLDLALLVDADEGTCG

SEQ ID NO: 110 - protein, 37 aa, synthetic construct; identical to SEQ ID NO: 17
  REGION 1..37: note = Description of Artificial Sequence: Synthetic polypeptide

SEQ ID NO: 111 - protein, 37 aa, synthetic construct; identical to SEQ ID NO: 35
  REGION 1..37: note = Description of Artificial Sequence: Synthetic polypeptide

SEQ ID NO: 112 - protein, 37 aa, synthetic construct; identical to SEQ ID NO: 34
  REGION 1..37: note = Description of Artificial Sequence: Synthetic polypeptide

SEQ ID NO: 113 - protein, 37 aa, synthetic construct; identical to SEQ ID NO: 32
  REGION 1..37: note = Description of Artificial Sequence: Synthetic polypeptide

SEQ ID NO: 114 - protein, 37 aa, synthetic construct; identical to SEQ ID NO: 93
  REGION 1..37: note = Description of Artificial Sequence: Synthetic polypeptide

SEQ ID NO: 115 - protein, 37 aa, synthetic construct; identical to SEQ ID NO: 94
  REGION 1..37: note = Description of Artificial Sequence: Synthetic polypeptide

SEQ ID NO: 116 - protein, 36 aa, synthetic construct; identical to SEQ ID NO: 19
  REGION 1..36: note = Description of Artificial Sequence: Synthetic polypeptide

SEQ ID NO: 117 - protein, 38 aa, synthetic construct
  REGION 1..38: note = Description of Artificial Sequence: Synthetic polypeptide
AAEQNPIYWARYADWLFTTPLLLLELALLVDADEGTCG

SEQ ID NO: 118 - protein, 38 aa, synthetic construct; identical to SEQ ID NO: 105
  REGION 1..38: note = Description of Artificial Sequence: Synthetic polypeptide

SEQ ID NO: 119 - protein, 39 aa, synthetic construct
  REGION 1..39: note = Description of Artificial Sequence: Synthetic polypeptide
AAEQNPIYWARYADWLFTTPLLLLELALLVDADEGTKCG

SEQ ID NO: 120 - protein, 38 aa, synthetic construct
  REGION 1..38: note = Description of Artificial Sequence: Synthetic polypeptide
GGEQNPIYWAQYADWLFTTPLLLLDLALLVDADEGTCG

SEQ ID NO: 121 - protein, 38 aa, synthetic construct; identical to SEQ ID NO: 107
  REGION 1..38: note = Description of Artificial Sequence: Synthetic polypeptide

SEQ ID NO: 122 - protein, 38 aa, synthetic construct; identical to SEQ ID NO: 108
  REGION 1..38: note = Description of Artificial Sequence: Synthetic polypeptide

SEQ ID NO: 123 - protein, 36 aa, synthetic construct; identical to SEQ ID NO: 19
  REGION 1..36: note = Description of Artificial Sequence: Synthetic polypeptide

SEQ ID NO: 124 - protein, 37 aa, synthetic construct; identical to SEQ ID NO: 34
  REGION 1..37: note = Description of Artificial Sequence: Synthetic polypeptide

SEQ ID NO: 125 - protein, 37 aa, synthetic construct; identical to SEQ ID NO: 35
  REGION 1..37: note = Description of Artificial Sequence: Synthetic polypeptide

SEQ ID NO: 126 - protein, 37 aa, synthetic construct; identical to SEQ ID NO: 18
  REGION 1..37: note = Description of Artificial Sequence: Synthetic polypeptide

SEQ ID NO: 127 - protein, 38 aa, synthetic construct; identical to SEQ ID NO: 108
  REGION 1..38: note = Description of Artificial Sequence: Synthetic polypeptide

SEQ ID NO: 128 - protein, 38 aa, synthetic construct; identical to SEQ ID NO: 107
  REGION 1..38: note = Description of Artificial Sequence: Synthetic polypeptide

SEQ ID NO: 129 - protein, 38 aa, synthetic construct; identical to SEQ ID NO: 120
  REGION 1..38: note = Description of Artificial Sequence: Synthetic polypeptide

SEQ ID NO: 130 - protein, 38 aa, synthetic construct; identical to SEQ ID NO: 109
  REGION 1..38: note = Description of Artificial Sequence: Synthetic polypeptide

SEQ ID NO: 131 - protein, 38 aa, synthetic construct
  REGION 1..38: note = Description of Artificial Sequence: Synthetic polypeptide
AAEQNPIYWARYADWLFTDLPLLLLDLLALLVDADEGT

SEQ ID NO: 132 - protein, 38 aa, synthetic construct; identical to SEQ ID NO: 28
  REGION 1..38: note = Description of Artificial Sequence: Synthetic polypeptide

SEQ ID NO: 133 - protein, 38 aa, synthetic construct; identical to SEQ ID NO: 27
  REGION 1..38: note = Description of Artificial Sequence: Synthetic polypeptide

SEQ ID NO: 134 - protein, 36 aa, synthetic construct; identical to SEQ ID NO: 31
  REGION 1..36: note = Description of Artificial Sequence: Synthetic polypeptide

SEQ ID NO: 135 - protein, 38 aa, synthetic construct; identical to SEQ ID NO: 109
  REGION 1..38: note = Description of Artificial Sequence: Synthetic polypeptide

SEQ ID NO: 136 - protein, 38 aa, synthetic construct; identical to SEQ ID NO: 107
  REGION 1..38: note = Description of Artificial Sequence: Synthetic polypeptide

SEQ ID NO: 137 - protein, 38 aa, synthetic construct; identical to SEQ ID NO: 108
  REGION 1..38: note = Description of Artificial Sequence: Synthetic polypeptide

SEQ ID NO: 138 - protein, 38 aa, synthetic construct; identical to SEQ ID NO: 29
  REGION 1..38: note = Description of Artificial Sequence: Synthetic polypeptide

SEQ ID NO: 139 - protein, 38 aa, synthetic construct
  REGION 1..38: note = Description of Artificial Sequence: Synthetic polypeptide
AAEQNPIYWARYADWLFTTPLLLLALALLVDADEGTCG

SEQ ID NO: 140 - protein, 39 aa, synthetic construct; identical to SEQ ID NO: 4
  REGION 1..39: note = Description of Artificial Sequence: Synthetic polypeptide
  REGION 37: note = Lys(rhodamine)
  REGION 38: note = Cys(phalloidin)

SEQ ID NO: 141 - protein, 39 aa, synthetic construct; identical to SEQ ID NO: 119
  SITE 37: note = Lys (rhodamine)
  SITE 38: note = Cys (phalloidin)

SEQ ID NO: 142 - protein, 38 aa, synthetic construct; identical to SEQ ID NO: 10
  SITE 37: note = Cys (phalloidin)

SEQ ID NO: 143 - protein, 38 aa, synthetic construct; identical to SEQ ID NO: 10
  SITE 37: note = Cys (phalloidin)

SEQ ID NO: 144 - protein, 38 aa, synthetic construct; identical to SEQ ID NO: 11
  REGION 1..38: note = Description of Artificial Sequence: Synthetic polypeptide

SEQ ID NO: 145 - protein, 35 aa, synthetic construct; identical to SEQ ID NO: 58
  REGION 1..35: note = Description of Artificial Sequence: Synthetic polypeptide

SEQ ID NO: 146 - protein, 37 aa, synthetic construct; identical to SEQ ID NO: 12
  REGION 1..37: note = Description of Artificial Sequence: Synthetic polypeptide

SEQ ID NO: 147 - protein, 36 aa, synthetic construct; identical to SEQ ID NO: 13
  REGION 1..36: note = Description of Artificial Sequence: Synthetic polypeptide

SEQ ID NO: 148 - protein, 36 aa, synthetic construct; identical to SEQ ID NO: 19
  REGION 1..36: note = Description of Artificial Sequence: Synthetic polypeptide

SEQ ID NO: 149 - protein, 37 aa, synthetic construct; identical to SEQ ID NO: 34
  REGION 1..37: note = Description of Artificial Sequence: Synthetic polypeptide

SEQ ID NO: 150 - protein, 37 aa, synthetic construct; identical to SEQ ID NO: 35
  REGION 1..37: note = Description of Artificial Sequence: Synthetic polypeptide

SEQ ID NO: 151 - protein, 38 aa, synthetic construct; identical to SEQ ID NO: 11
  REGION 1..38: note = Description of Artificial Sequence: Synthetic polypeptide

SEQ ID NO: 152 - protein, 38 aa, synthetic construct; identical to SEQ ID NO: 10
  SITE 37: note = Cys (phalloidin)

SEQ ID NO: 153 - protein, 39 aa, synthetic construct; identical to SEQ ID NO: 119
  VARIANT 37: note = Lys (rhodamine)
  VARIANT 38: note = Cys (phalloidin)

SEQ ID NO: 154 - protein, 39 aa, synthetic construct; identical to SEQ ID NO: 4
  SITE 37: note = Lys (rhodamine)
  SITE 38: note = Cys (phalloidin)

SEQ ID NO: 155 - protein, 37 aa, synthetic construct; identical to SEQ ID NO: 93
  REGION 1..37: note = Description of Artificial Sequence: Synthetic polypeptide

SEQ ID NO: 156 - protein, 37 aa, synthetic construct; identical to SEQ ID NO: 94
  REGION 1..37: note = Description of Artificial Sequence: Synthetic polypeptide

SEQ ID NO: 157 - protein, 37 aa, synthetic construct; identical to SEQ ID NO: 34
  REGION 1..37: note = Description of Artificial Sequence: Synthetic polypeptide

SEQ ID NO: 158 - protein, 37 aa, synthetic construct; identical to SEQ ID NO: 35
  REGION 1..37: note = Description of Artificial Sequence: Synthetic polypeptide

SEQ ID NO: 159 - protein, 37 aa, synthetic construct; identical to SEQ ID NO: 32
  REGION 1..37: note = Description of Artificial Sequence: Synthetic polypeptide

SEQ ID NO: 160 - protein, 37 aa, synthetic construct; identical to SEQ ID NO: 32
  REGION 1..37: note = Description of Artificial Sequence: Synthetic polypeptide

SEQ ID NO: 161 - protein, 37 aa, synthetic construct; identical to SEQ ID NO: 94
  REGION 1..37: note = Description of Artificial Sequence: Synthetic polypeptide

SEQ ID NO: 162 - protein, 37 aa, synthetic construct; identical to SEQ ID NO: 93
  REGION 1..37: note = Description of Artificial Sequence: Synthetic polypeptide

SEQ ID NO: 163 - protein, 37 aa, synthetic construct; identical to SEQ ID NO: 34
  REGION 1..37: note = Description of Artificial Sequence: Synthetic polypeptide

SEQ ID NO: 164 - protein, 37 aa, synthetic construct; identical to SEQ ID NO: 35
  REGION 1..37: note = Description of Artificial Sequence: Synthetic polypeptide

SEQ ID NO: 165 - protein, 37 aa, synthetic construct; identical to SEQ ID NO: 35
  REGION 1..37: note = Description of Artificial Sequence: Synthetic polypeptide

SEQ ID NO: 166 - protein, 37 aa, synthetic construct; identical to SEQ ID NO: 34
  REGION 1..37: note = Description of Artificial Sequence: Synthetic polypeptide

SEQ ID NO: 167 - protein, 37 aa, synthetic construct; identical to SEQ ID NO: 32
  REGION 1..37: note = Description of Artificial Sequence: Synthetic polypeptide

SEQ ID NO: 168 - protein, 37 aa, synthetic construct; identical to SEQ ID NO: 93
  REGION 1..37: note = Description of Artificial Sequence: Synthetic polypeptide

SEQ ID NO: 169 - protein, 37 aa, synthetic construct; identical to SEQ ID NO: 94
  REGION 1..37: note = Description of Artificial Sequence: Synthetic polypeptide

SEQ ID NO: 170 - protein, 36 aa, synthetic construct; identical to SEQ ID NO: 19
  REGION 1..36: note = Description of Artificial Sequence: Synthetic polypeptide

SEQ ID NO: 171 - protein, 37 aa, synthetic construct; identical to SEQ ID NO: 34
  REGION 1..37: note = Description of Artificial Sequence: Synthetic polypeptide

SEQ ID NO: 172 - protein, 37 aa, synthetic construct; identical to SEQ ID NO: 32
  REGION 1..37: note = Description of Artificial Sequence: Synthetic polypeptide

SEQ ID NO: 173 - protein, 37 aa, synthetic construct; identical to SEQ ID NO: 93
  REGION 1..37: note = Description of Artificial Sequence: Synthetic polypeptide

SEQ ID NO: 174 - protein, 37 aa, synthetic construct; identical to SEQ ID NO: 94
  REGION 1..37: note = Description of Artificial Sequence: Synthetic polypeptide

SEQ ID NO: 175 - protein, 37 aa, synthetic construct; identical to SEQ ID NO: 35
  REGION 1..37: note = Description of Artificial Sequence: Synthetic polypeptide

SEQ ID NO: 176 - protein, 37 aa, synthetic construct; identical to SEQ ID NO: 94
  REGION 1..37: note = Description of Artificial Sequence: Synthetic polypeptide

SEQ ID NO: 177 - protein, 37 aa, synthetic construct; identical to SEQ ID NO: 32
  REGION 1..37: note = Description of Artificial Sequence: Synthetic polypeptide

SEQ ID NO: 178 - protein, 37 aa, synthetic construct; identical to SEQ ID NO: 93
  REGION 1..37: note = Description of Artificial Sequence: Synthetic polypeptide

SEQ ID NO: 179 - protein, 37 aa, synthetic construct; identical to SEQ ID NO: 34
  REGION 1..37: note = Description of Artificial Sequence: Synthetic polypeptide

SEQ ID NO: 180 - protein, 37 aa, synthetic construct; identical to SEQ ID NO: 35
  REGION 1..37: note = Description of Artificial Sequence: Synthetic polypeptide

SEQ ID NO: 181 - protein, 36 aa, synthetic construct; identical to SEQ ID NO: 31
  REGION 1..36: note = Description of Artificial Sequence: Synthetic polypeptide

SEQ ID NO: 182 - protein, 38 aa, synthetic construct; identical to SEQ ID NO: 30
  REGION 1..38: note = Description of Artificial Sequence: Synthetic polypeptide

SEQ ID NO: 183 - protein, 38 aa, synthetic construct; identical to SEQ ID NO: 29
  REGION 1..38: note = Description of Artificial Sequence: Synthetic polypeptide

SEQ ID NO: 184 - protein, 38 aa, synthetic construct; identical to SEQ ID NO: 107
  REGION 1..38: note = Description of Artificial Sequence: Synthetic polypeptide

SEQ ID NO: 185 - protein, 38 aa, synthetic construct; identical to SEQ ID NO: 108
  REGION 1..38: note = Description of Artificial Sequence: Synthetic polypeptide

SEQ ID NO: 186 - protein, 38 aa, synthetic construct; identical to SEQ ID NO: 109
  REGION 1..38: note = Description of Artificial Sequence: Synthetic polypeptide

SEQ ID NO: 187 - protein, 38 aa, synthetic construct; identical to SEQ ID NO: 28
  REGION 1..38: note = Description of Artificial Sequence: Synthetic polypeptide

SEQ ID NO: 188 - protein, 38 aa, synthetic construct; identical to SEQ ID NO: 27
  REGION 1..38: note = Description of Artificial Sequence: Synthetic polypeptide

SEQ ID NO: 189 - protein, 38 aa, synthetic construct; identical to SEQ ID NO: 27
  REGION 1..38: note = Description of Artificial Sequence: Synthetic polypeptide

SEQ ID NO: 190 - protein, 38 aa, synthetic construct; identical to SEQ ID NO: 28
  REGION 1..38: note = Description of Artificial Sequence: Synthetic polypeptide

SEQ ID NO: 191 - protein, 38 aa, synthetic construct; identical to SEQ ID NO: 120
  REGION 1..38: note = Description of Artificial Sequence: Synthetic polypeptide

SEQ ID NO: 192 - protein, 38 aa, synthetic construct; identical to SEQ ID NO: 107
  REGION 1..38: note = Description of Artificial Sequence: Synthetic polypeptide

SEQ ID NO: 193 - protein, 38 aa, synthetic construct; identical to SEQ ID NO: 108
  REGION 1..38: note = Description of Artificial Sequence: Synthetic polypeptide

SEQ ID NO: 194 - protein, 38 aa, synthetic construct; identical to SEQ ID NO: 107
  REGION 1..38: note = Description of Artificial Sequence: Synthetic polypeptide

SEQ ID NO: 195 - protein, 38 aa, synthetic construct; identical to SEQ ID NO: 108
  REGION 1..38: note = Description of Artificial Sequence: Synthetic polypeptide

SEQ ID NO: 196 - protein, 38 aa, synthetic construct; identical to SEQ ID NO: 120
  REGION 1..38: note = Description of Artificial Sequence: Synthetic polypeptide

SEQ ID NO: 197 - protein, 38 aa, synthetic construct; identical to SEQ ID NO: 109
  REGION 1..38: note = Description of Artificial Sequence: Synthetic polypeptide

SEQ ID NO: 198 - protein, 38 aa, synthetic construct; identical to SEQ ID NO: 108
  REGION 1..38: note = Description of Artificial Sequence: Synthetic polypeptide

SEQ ID NO: 199 - protein, 38 aa, synthetic construct; identical to SEQ ID NO: 107
  REGION 1..38: note = Description of Artificial Sequence: Synthetic polypeptide

SEQ ID NO: 200 - protein, 38 aa, synthetic construct; identical to SEQ ID NO: 120
  REGION 1..38: note = Description of Artificial Sequence: Synthetic polypeptide

SEQ ID NO: 201 - protein, 38 aa, synthetic construct; identical to SEQ ID NO: 109
  REGION 1..38: note = Description of Artificial Sequence: Synthetic polypeptide

SEQ ID NO: 202 - protein, 39 aa, synthetic construct; identical to SEQ ID NO: 119
  REGION 1..39: note = Description of Artificial Sequence: Synthetic polypeptide

SEQ ID NO: 203 - protein, 6 aa, synthetic construct
  SITE 4: note = Lys (rhodamine)
  SITE 5: note = Cys (phalloidin)
EGTKCG

SEQ ID NO: 204 - protein, 39 aa, synthetic construct; identical to SEQ ID NO: 4
  VARIANT 37: note = Lys (rhodamine)
  VARIANT 38: note = Cys (phalloidin)

SEQ ID NO: 205 - protein, 37 aa, synthetic construct; identical to SEQ ID NO: 12
  REGION 1..37: note = Description of Artificial Sequence: Synthetic polypeptide

SEQ ID NO: 206 - protein, 38 aa, synthetic construct; identical to SEQ ID NO: 10
  SITE 37: note = Cys (phalloidin)

SEQ ID NO: 207 - protein, 39 aa, synthetic construct; identical to SEQ ID NO: 4
  SITE 37: note = Lys (rhodamine)
  SITE 38: note = Cys (phalloidin)

SEQ ID NO: 208 - protein, 39 aa, synthetic construct; identical to SEQ ID NO: 119
  VARIANT 37: note = Lys (rhodamine)
  VARIANT 38: note = Cys (phalloidin)

SEQ ID NO: 209 - protein, 38 aa, synthetic construct; identical to SEQ ID NO: 131
  REGION 1..38: note = Description of Artificial Sequence: Synthetic polypeptide

SEQ ID NO: 210 - protein, 38 aa, synthetic construct; identical to SEQ ID NO: 109
  REGION 1..38: note = Description of Artificial Sequence: Synthetic polypeptide

SEQ ID NO: 211 - protein, 38 aa, synthetic construct; identical to SEQ ID NO: 107
  REGION 1..38: note = Description of Artificial Sequence: Synthetic polypeptide

SEQ ID NO: 212 - protein, 38 aa, synthetic construct; identical to SEQ ID NO: 108
  REGION 1..38: note = Description of Artificial Sequence: Synthetic polypeptide

SEQ ID NO: 213 - protein, 38 aa, synthetic construct; identical to SEQ ID NO: 29
  REGION 1..38: note = Description of Artificial Sequence: Synthetic polypeptide

SEQ ID NO: 214 - protein, 38 aa, synthetic construct; identical to SEQ ID NO: 104
  REGION 1..38: note = Description of Artificial Sequence: Synthetic polypeptide

SEQ ID NO: 215 - protein, 38 aa, synthetic construct; identical to SEQ ID NO: 105
  REGION 1..38: note = Description of Artificial Sequence: Synthetic polypeptide

SEQ ID NO: 216 - protein, 38 aa, synthetic construct; identical to SEQ ID NO: 139
  REGION 1..38: note = Description of Artificial Sequence: Synthetic polypeptide

SEQ ID NO: 217 - protein, 38 aa, synthetic construct; identical to SEQ ID NO: 117
  REGION 1..38: note = Description of Artificial Sequence: Synthetic polypeptide

SEQ ID NO: 218 - protein, 38 aa, synthetic construct; identical to SEQ ID NO: 105
  REGION 1..38: note = Description of Artificial Sequence: Synthetic polypeptide

SEQ ID NO: 219 - protein, 39 aa, synthetic construct; identical to SEQ ID NO: 119
  SITE 37: note = Lys (rhodamine)
  SITE 38: note = Cys (phalloidin)

SEQ ID NO: 220 - protein, 37 aa, synthetic construct; identical to SEQ ID NO: 18
  REGION 1..37: note = Description of Artificial Sequence: Synthetic polypeptide

SEQ ID NO: 221 - protein, 37 aa, synthetic construct; identical to SEQ ID NO: 17
  REGION 1..37: note = Description of Artificial Sequence: Synthetic polypeptide

SEQ ID NO: 222 - protein, 36 aa, synthetic construct; identical to SEQ ID NO: 22
  REGION 1..36: note = Description of Artificial Sequence: Synthetic polypeptide

SEQ ID NO: 223 - protein, 22 aa, synthetic construct
  REGION 1..22: note = Description of Artificial Sequence: Synthetic peptide
AEQNPIYFARYADLLFPTTLAW

SEQ ID NO: 224 - protein, 22 aa, synthetic construct
  REGION 1..22: note = Description of Artificial Sequence: Synthetic peptide
AEQNPIYWARYADLLFPTTLAF

SEQ ID NO: 225 - protein, 22 aa, synthetic construct
  REGION 1..22: note = Description of Artificial Sequence: Synthetic peptide
AEQNPIYWARYADLLFPTTLAW

SEQ ID NO: 226 - protein, 35 aa, synthetic construct; identical to SEQ ID NO: 58
  REGION 1..35: note = Description of Artificial Sequence: Synthetic polypeptide

SEQ ID NO: 227 - protein, 36 aa, synthetic construct; identical to SEQ ID NO: 8
  REGION 1..36: note = Description of Artificial Sequence: Synthetic polypeptide

SEQ ID NO: 228 - protein, 38 aa, synthetic construct; identical to SEQ ID NO: 10
  REGION 1..38: note = Description of Artificial Sequence: Synthetic polypeptide

SEQ ID NO: 229 - protein, 39 aa, synthetic construct; identical to SEQ ID NO: 4
  REGION 1..39: note = Description of Artificial Sequence: Synthetic polypeptide

SEQ ID NO: 230 - protein, 36 aa, synthetic construct; identical to SEQ ID NO: 16
  REGION 1..36: note = Description of Artificial Sequence: Synthetic polypeptide

SEQ ID NO: 231 - DNA, 18 bp, synthetic construct
  source 1..18: mol_type = other DNA
cctcttacctcagttaca

SEQ ID NO: 232 - DNA, 18 bp, synthetic construct
  source 1..18: mol_type = other DNA
cctcttacctcagttaca

SEQ ID NO: 233 - DNA, 18 bp, synthetic construct
  source 1..18: mol_type = other DNA
cctcttacctcagttaca

SEQ ID NO: 234 - DNA, 18 bp, synthetic construct
  source 1..18: mol_type = other DNA
cctcttacctcagttaca

SEQ ID NO: 235 - DNA, 18 bp, synthetic construct
  source 1..18: mol_type = other DNA
cctctgacctcatttaca

SEQ ID NO: 236 - DNA, 18 bp, synthetic construct
  source 1..18: mol_type = other DNA
cctcttacctcagttaca

SEQ ID NO: 237 - DNA, 18 bp, synthetic construct
  source 1..18: mol_type = other DNA
cctctgacctcatttaca

SEQ ID NO: 238 - DNA, 18 bp, synthetic construct
  source 1..18: mol_type = other DNA
cctcttacctcagttaca

SEQ ID NO: 239 - protein, 38 aa, unidentified; identical to SEQ ID NO: 10
  REGION 1..38: note = Description of Unknown: pH-sensitive membrane polypeptide

SEQ ID NO: 240 - protein, 31 aa, synthetic construct
  REGION 1..31: note = Description of Artificial Sequence: Synthetic polypeptide
AEDQNPYWARYDWLFTTPLLLLDLALLVDCG

SEQ ID NO: 241 - protein, 32 aa, synthetic construct; identical to SEQ ID NO: 42
  REGION 1..32: note = Description of Artificial Sequence: Synthetic polypeptide

SEQ ID NO: 242 - protein, 36 aa, synthetic construct; identical to SEQ ID NO: 60
  REGION 1..36: note = Description of Artificial Sequence: Synthetic polypeptide

SEQ ID NO: 243 - protein, 35 aa, synthetic construct; identical to SEQ ID NO: 58
  REGION 1..35: note = Description of Artificial Sequence: Synthetic polypeptide

SEQ ID NO: 244 - protein, 35 aa, synthetic construct; identical to SEQ ID NO: 7
  REGION 1..35: note = Description of Artificial Sequence: Synthetic polypeptide

SEQ ID NO: 245 - protein, 31 aa, synthetic construct; identical to SEQ ID NO: 72
  REGION 1..31: note = Description of Artificial Sequence: Synthetic polypeptide

SEQ ID NO: 246 - protein, 31 aa, synthetic construct
  REGION 1..31: note = Description of Artificial Sequence: Synthetic polypeptide
AEDQNDPYWARYADWLFTTPLLLLDLALLVG

SEQ ID NO: 247 - protein, 34 aa, synthetic construct; identical to SEQ ID NO: 36
  REGION 1..34: note = Description of Artificial Sequence: Synthetic polypeptide

SEQ ID NO: 248 - protein, 34 aa, synthetic construct
  REGION 1..34: note = Description of Artificial Sequence: Synthetic polypeptide
AEQNPIYFARYADWLFTTPLLLLDLALLVDADET

SEQ ID NO: 249 - protein, 34 aa, synthetic construct; identical to SEQ ID NO: 38
  REGION 1..34: note = Description of Artificial Sequence: Synthetic polypeptide

SEQ ID NO: 250 - protein, 36 aa, synthetic construct; identical to SEQ ID NO: 60
  REGION 1..36: note = Description of Artificial Sequence: Synthetic polypeptide

SEQ ID NO: 251 - protein, 34 aa, synthetic construct
  REGION 1..34: note = Description of Artificial Sequence: Synthetic polypeptide
AEDQNPIYWARYADWLFTTPLLLLDLALLVDCGT

SEQ ID NO: 252 - protein, 35 aa, synthetic construct
  REGION 1..35: note = Description of Artificial Sequence: Synthetic polypeptide
AEDQNDPIYWARYADWLFTTPLLLLELALLVECGT

SEQ ID NO: 253 - protein, 25 aa, synthetic construct
  REGION 1..25: note = Description of Artificial Sequence: Synthetic peptide
ACEEQNPWARYLEWLFPTETLLLEL

SEQ ID NO: 254 - protein, 35 aa, synthetic construct; identical to SEQ ID NO: 7
  REGION 1..35: note = Description of Artificial Sequence: Synthetic polypeptide

SEQ ID NO: 255 - protein, 32 aa, synthetic construct; identical to SEQ ID NO: 39
  REGION 1..32: note = Description of Artificial Sequence: Synthetic polypeptide

SEQ ID NO: 256 - protein, 32 aa, synthetic construct; identical to SEQ ID NO: 76
  REGION 1..32: note = Description of Artificial Sequence: Synthetic polypeptide

SEQ ID NO: 257 - protein, 35 aa, synthetic construct
  REGION 1..35: note = Description of Artificial Sequence: Synthetic polypeptide
AEQNPIYWARYADWLFTTPLLLLDLALLVDADEGC

SEQ ID NO: 258 - protein, 31 aa, synthetic construct
  REGION 1..31: note = Description of Artificial Sequence: Synthetic polypeptide
AEDQNPYWARYADWLFTTPLLLLDLALLVDC

SEQ ID NO: 259 - protein, 31 aa, synthetic construct
  REGION 1..31: note = Description of Artificial Sequence: Synthetic polypeptide
AEDQNPYWARYADWLFTTPLLLLELALLVEC

SEQ ID NO: 260 - protein, 36 aa, synthetic construct; identical to SEQ ID NO: 13
  REGION 1..36: note = Description of Artificial Sequence: Synthetic polypeptide

SEQ ID NO: 261 - protein, 32 aa, synthetic construct; identical to SEQ ID NO: 40
  REGION 1..32: note = Description of Artificial Sequence: Synthetic polypeptide

SEQ ID NO: 262 - protein, 30 aa, synthetic construct; identical to SEQ ID NO: 44
  REGION 1..30: note = Description of Artificial Sequence: Synthetic polypeptide

SEQ ID NO: 263 - protein, 27 aa, synthetic construct
  REGION 1..27: note = Description of Artificial Sequence: Synthetic peptide
ACDDQNPWRAYLDLLFPTDTLLLDLLW

SEQ ID NO: 264 - protein, 27 aa, synthetic construct
  REGION 1..27: note = Description of Artificial Sequence: Synthetic peptide
ACEEQNPWRAYLELLFPTETLLLELLW

SEQ ID NO: 265 - protein, 25 aa, synthetic construct
  REGION 1..25: note = Description of Artificial Sequence: Synthetic peptide
ACDDQNPWARYLDWLFPTDTLLLDL

SEQ ID NO: 266 - protein, 24 aa, synthetic construct
  REGION 1..24: note = Description of Artificial Sequence: Synthetic peptide
CDNNNPWRAYLDLLFPTDTLLLDW

SEQ ID NO: 267 - protein, 25 aa, synthetic construct
  REGION 1..25: note = Description of Artificial Sequence: Synthetic peptide
ACEEQNPWARYLEWLFPTETLLLEL

SEQ ID NO: 268 - protein, 24 aa, synthetic construct
  REGION 1..24: note = Description of Artificial Sequence: Synthetic peptide
CEEQQPWAQYLELLFPTETLLLEW

SEQ ID NO: 269 - protein, 24 aa, synthetic construct
  REGION 1..24: note = Description of Artificial Sequence: Synthetic peptide
CEEQQPWRAYLELLFPTETLLLEW

SEQ ID NO: 270 - protein, 24 aa, synthetic construct
  REGION 1..24: note = Description of Artificial Sequence: Synthetic peptide
ACEDQNPWARYADWLFPTTLLLLD

SEQ ID NO: 271 - protein, 24 aa, synthetic construct
  REGION 1..24: note = Description of Artificial Sequence: Synthetic peptide
ACEEQNPWARYAEWLFPTTLLLLE

SEQ ID NO: 272 - protein, 22 aa, synthetic construct
  REGION 1..22: note = Description of Artificial Sequence: Synthetic peptide
ACEDQNPWARYADLLFPTTLAW

SEQ ID NO: 273 - protein, 22 aa, synthetic construct
  REGION 1..22: note = Description of Artificial Sequence: Synthetic peptide
ACEEQNPWARYAELLFPTTLAW

SEQ ID NO: 274 - protein, 34 aa, synthetic construct; identical to SEQ ID NO: 46
  REGION 1..34: note = Description of Artificial Sequence: Synthetic polypeptide

SEQ ID NO: 275 - protein, 37 aa, synthetic construct
  REGION 1..37: note = Description of Artificial Sequence: Synthetic polypeptide
CDDDDDNPNYWARYANWLFTTPLLLLNGALLVEAEET

SEQ ID NO: 276 - protein, 37 aa, synthetic construct
  REGION 1..37: note = Description of Artificial Sequence: Synthetic polypeptide
CDDDDDNPNYWARYAPWLFTTPLLLLPGALLVEAEET

SEQ ID NO: 277 - protein, 36 aa, synthetic construct; identical to SEQ ID NO: 60
  REGION 1..36: note = Description of Artificial Sequence: Synthetic polypeptide

SEQ ID NO: 278 - protein, 37 aa, synthetic construct
  REGION 1..37: note = Description of Artificial Sequence: Synthetic polypeptide
AKEQNPIYWARYADWLFTTPLLLLDLALLVDADEGTG

SEQ ID NO: 279 - protein, 36 aa, synthetic construct
  REGION 1..36: note = Description of Artificial Sequence: Synthetic polypeptide
ACEQNPIYWARYANWLFTTPLLLLNLALLVDADEGT

SEQ ID NO: 280 - protein, 36 aa, synthetic construct
  REGION 1..36: note = Description of Artificial Sequence: Synthetic polypeptide
DDDEDNPIYWARYADWLFTTPLLLLHGALLVDADET

SEQ ID NO: 281 - protein, 36 aa, synthetic construct; identical to SEQ ID NO: 280
  REGION 1..36: note = Description of Artificial Sequence: Synthetic polypeptide

SEQ ID NO: 282 - protein, 37 aa, synthetic construct
  REGION 1..37: note = Description of Artificial Sequence: Synthetic polypeptide
CDDDEDNPIYWARYAHWLFTTPLLLLHGALLVDADET

SEQ ID NO: 283 - protein, 37 aa, synthetic construct
  REGION 1..37: note = Description of Artificial Sequence: Synthetic polypeptide
DDDEDNPIYWARYAHWLFTTPLLLLHGALLVDADEGT

SEQ ID NO: 284 - protein, 37 aa, synthetic construct
  REGION 1..37: note = Description of Artificial Sequence: Synthetic polypeptide
DDDEDNPIYWARYAHWLFTTPLLLLHGALLVNADEGT

SEQ ID NO: 285 - protein, 37 aa, synthetic construct
  REGION 1..37: note = Description of Artificial Sequence: Synthetic polypeptide
DDDEDNPIYWARYAHWLFTTPLLLLHGALLVNANEGT

SEQ ID NO: 286 - protein, 32 aa, synthetic construct; identical to SEQ ID NO: 76
  REGION 1..32: note = Description of Artificial Sequence: Synthetic polypeptide

SEQ ID NO: 287 - protein, 31 aa, synthetic construct
  REGION 1..31: note = Description of Artificial Sequence: Synthetic polypeptide
AEDQNPYWARYADWLFTTPLLLLELALLVCG

SEQ ID NO: 288 - protein, 28 aa, synthetic construct
  REGION 1..28: note = Description of Artificial Sequence: Synthetic peptide
AKDDQNPWRAYLDLLFPTDTLLLDLLWC

SEQ ID NO: 289 - protein, 27 aa, synthetic construct
  REGION 1..27: note = Description of Artificial Sequence: Synthetic peptide
ACEEQNPWRAYLELLFPTETLLLELLW

SEQ ID NO: 290 - protein, 25 aa, synthetic construct
  REGION 1..25: note = Description of Artificial Sequence: Synthetic peptide
ACDDQNPWARYLDWLFPTDTLLLDL

SEQ ID NO: 291 - protein, 24 aa, synthetic construct
  REGION 1..24: note = Description of Artificial Sequence: Synthetic peptide
CDNNNPWRAYLDLLFPTDTLLLDW

SEQ ID NO: 292 - protein, 24 aa, synthetic construct
  REGION 1..24: note = Description of Artificial Sequence: Synthetic peptide
CEEQQPWAQYLELLFPTETLLLEW

SEQ ID NO: 293 - protein, 24 aa, synthetic construct
  REGION 1..24: note = Description of Artificial Sequence: Synthetic peptide
CEEQQPWRAYLELLFPTETLLLEW

SEQ ID NO: 294 - protein, 37 aa, synthetic construct; identical to SEQ ID NO: 275
  REGION 1..37: note = Description of Artificial Sequence: Synthetic polypeptide

SEQ ID NO: 295 - protein, 36 aa, synthetic construct
  REGION 1..36: note = Description of Artificial Sequence: Synthetic polypeptide
CDDDDDNPNYWARYAPWLFTTPLLLLPGALLVEAEE

SEQ ID NO: 296 - protein, 22 aa, synthetic construct
  REGION 1..22: note = Description of Artificial Sequence: Synthetic peptide
AEQNPIYFARYADLLFPTTLAW

SEQ ID NO: 297 - protein, 22 aa, synthetic construct
  REGION 1..22: note = Description of Artificial Sequence: Synthetic peptide
AEQNPIYWARYADLLFPTTLAF

SEQ ID NO: 298 - protein, 22 aa, synthetic construct
  REGION 1..22: note = Description of Artificial Sequence: Synthetic peptide
AEQNPIYWARYADLLFPTTLAW

SEQ ID NO: 299 - protein, 20 aa, synthetic construct
  REGION 1..20: note = Description of Artificial Sequence: Synthetic peptide
KEDQNPWARYADLLFPTTLW

SEQ ID NO: 300 - protein, 24 aa, synthetic construct
  REGION 1..24: note = Description of Artificial Sequence: Synthetic peptide
ACEEQNPQAEYAEWLFPTTLLLLE

SEQ ID NO: 301 - protein, 25 aa, synthetic construct
  REGION 1..25: note = Description of Artificial Sequence: Synthetic peptide
AAEEQNPWARYLEWLFPTETLLLEL

SEQ ID NO: 302 - protein, 25 aa, synthetic construct
  REGION 1..25: note = Description of Artificial Sequence: Synthetic peptide
AKEEQNPWARYLEWLFPTETLLLEL

SEQ ID NO: 303 - protein, 38 aa, unidentified
  REGION 1..38: note = Description of Unknown: Wild-type pH-sensitive membrane polypeptide
AAEQNPIYWARYADWLFTTPLLLLDLALLVDADEGTGG

SEQ ID NO: 304 - protein, 38 aa, synthetic construct
  VARIANT 1: note = Ala or Asp
  VARIANT 2: note = Ala, Asp or Cys
  VARIANT 4: note = Gln or Asp
  VARIANT 11: note = Arg or Gln
  VARIANT 12: note = Tyr or Asp
  VARIANT 13: note = Ala, Asp or Tyr
  VARIANT 14: note = Asp, Asn, Flu, His, Lys, Ala or Trp
  VARIANT 15: note = Trp or Asp
  VARIANT 19: note = Thr or Asp
  VARIANT 20: note = Pro, Gly or Ala
  VARIANT 24: note = Leu or Asp
  VARIANT 25: note = Asp, Leu, Asn, Glu, His, Lys or Ala
  VARIANT 26: note = Leu, Asp or Gly
  VARIANT 31: note = Asp or Asn
  VARIANT 33: note = Asp or Asn
  VARIANT 34: note = Glu or Gln
  VARIANT 35: note = Gly or Cys
  VARIANT 37: note = Gly or Cys
XXEXNPIYWAXXXXXLFTXXLLLXXXALLVXAXXXTXG

SEQ ID NO: 305 - protein, 42 aa, synthetic construct
  SITE 1: note = May or may not be present
  SITE 21: note = May or may not be present
  SITE 29: note = May or may not be present
  SITE 40: note = May or may not be present
DAAEQNPIYWARYADWLFTTLPLLLLDLLALLVDADEGTKGG

SEQ ID NO: 306 - protein, 38 aa, unidentified
  REGION 1..38: note = Description of Unknown: Wild-type pH-sensitive membrane polypeptide
GGEQNPIYWARYADWLFTTPLLLLDLALLVDADEGTGG

SEQ ID NO: 307 - protein, 38 aa, synthetic construct
XXEXNPIYWAXXXXXLFTXXLLLXXXALLVXAXXXTGG

SEQ ID NO: 308 - protein, 44 aa, synthetic construct
  SITE 1: note = May or may not be present
  SITE 7: note = May or may not be present
  SITE 22: note = May or may not be present
  SITE 30: note = May or may not be present
  SITE 40: note = May or may not be present
  SITE 42: note = Lys, Cys or absent
DGGEQNDPIYWARYADWLFTTLPLLLLDLLALLVDADEGCTXGG

SEQ ID NO: 309 - protein, 38 aa, synthetic construct; identical to SEQ ID NO: 10
  MOD_RES 37: note = S-S linker attached to the nitrogen of amino-phalloidin

SEQ ID NO: 310 - protein, 31 aa, synthetic construct; identical to SEQ ID NO: 240
  MOD_RES 1: note = S-S linker attached to 2-amino phalloidin
  MOD_RES 29: note = Lys residue 29 modified with an alkyl linker attached to rhodamine
  MOD_RES 30: note = Alanine residue 1 modified with a COCH3 group

SEQ ID NO: 311 - protein, 34 aa, synthetic construct
GLAGLAGLLGLEGLLGLPLGLLEGLWLGLELEGN

(19) United States
(12) Patent Application Publication
(10) Pub. No.: US 2024/0067616 A1
(43) Pub. Date: Feb. 29, 2024

(54) PEPTIDE CONJUGATES OF MICROTUBULE-TARGETING AGENTS AS THERAPEUTICS

(71) Applicant: Cybrexa 3, Inc., New Haven, CT (US)

(72) Inventors: Daniel Richard Marshall, New Haven, CT (US); Johanna Marie Csengery, New Fairfield, CT (US); Robert John Maguire, New Haven, CT (US); Robert A. Volkmann, Mystic, CT (US)

(21) Appl. No.: 18/152,987

(22) Filed: Jan. 11, 2023

Related U.S. Application Data
(63) Continuation of application No. 16/924,445, filed on Jul. 9, 2020, now Pat. No. 11,555,019.
(60) Provisional application No. 63/041,324, filed on Jun. 19, 2020.
(60) Provisional application No. 62/872,638, filed on Jul. 10, 2019.

(51) Int. Cl. C07D 265/12; A61K 47/64

(52) U.S. Cl. CPC C07D 265/12; A61K 47/64

(57) ABSTRACT

The present invention relates to peptide conjugates of microtubule-targeting agents such as maytansinoid derivatives which are useful for the treatment of diseases such as cancer.

### CROSS-REFERENCE TO RELATED APPLICATIONS

This application is a continuation of U.S. application Ser. No. 16/924,445, filed Jul. 9, 2020 (now U.S. Pat. No. 11,555,019), which claims the benefit of U.S. Provisional Application Ser. No. 63/041,324 filed Jun. 19, 2020 and U.S. Provisional Application Ser. No. 62/872,638 filed Jul. 10, 2019, which are incorporated herein by reference in their entirety.

### FIELD OF THE INVENTION

The present invention relates to peptide conjugates of microtubule-targeting agents such as maytansinoid derivatives which are useful for the treatment of diseases such as cancer.

### SEQUENCE LISTING

This application contains a Sequence Listing that has been submitted electronically as an XML file named 43236-0008002_SL_ST26.xml. The XML file, created on Feb. 24, 2023, is 416,890 bytes in size. The material in the XML file is hereby incorporated by reference in its entirety.

### BACKGROUND OF THE INVENTION

Cancer is a group of diseases characterized by aberrant control of cell growth. The annual incidence of cancer is estimated to be in excess of 1.6 million in the United States alone. While surgery, radiation, chemotherapy, and hormones are used to treat cancer, it remains the second leading cause of death in the U.S. It is estimated that about 600,000 Americans will die from cancer each year.

Treatment of cancer in humans by systemic administration of pharmaceutical agents often functions by slowing or terminating the uncontrolled replication that is a characteristic of cancer cells. One class of such agents is microtubule-targeting agents. Cell division requires formation of an intact mitotic spindle apparatus, composed of microtubules undergoing random length changes. The random length changes of microtubules is referred to as dynamic instability. Disruption of the dynamic instability of microtubules can lead to the suppression of further cell division. Drugs that target microtubules to suppress dynamic instability are currently used in the clinic as effective anticancer agents for a wide variety of cancers. See Lopus, M, Cancer Lett., 2011, 307(2): 113-118.

The maytansinoids, (e.g., mertansine, DM1, or DM4) are a class of microtubule-targeting agents that have emerged as potential clinical chemotherapeutics. See Lopus, M, Cancer Lett., 2011, 307(2): 113-118; and Widdison, W., J. Med. Chem. 2006, 49:4392-4408 Although DM1 has been shown to be effective in the treatment of several types of cancer, including lymphoma and breast cancer, toxic side effects such as peripheral neuropathy have hindered the clinical development of tubulin-targeting agents such as the maytansinoids. Preferential delivery of maytansinoid compounds, such as DM1, to diseased tissues could avoid these serious side effects. Thus, there is a need for more selective delivery of maytansinoid compounds to diseased tissue.

### SUMMARY

The present disclosure provides, inter alia, a compound of Formula (I):

R²-L-R¹ (I)

or a pharmaceutically acceptable salt thereof, wherein constituent variables are defined herein.

The present disclosure further provides a pharmaceutical composition comprising a compound of the disclosure, or a pharmaceutically acceptable salt thereof, and at least one pharmaceutically acceptable carrier or excipient.

The present disclosure also provides methods of treating a disease or condition (e.g., cancer) by administering to a human or other mammal in need of such treatment a therapeutically effective amount of a compound of the disclosure. In some embodiments, the disease or condition is characterized by acidic or hypoxic diseased tissues.

The present disclosure also provides use of a compound described herein in the manufacture of a medicament for use in therapy. The present disclosure also provides the compounds described herein for use in therapy.

The present disclosure also provides methods for synthesizing the compounds of the disclosure and intermediates useful in these methods.

### BRIEF DESCRIPTION OF THE DRAWINGS

FIG. 1 shows a plot of the effect of free DM4 and Compound 5 on in vitro β-tubulin polymerization (in terms of relative fluorescence units) at various concentrations.

FIG. 2 depicts the kinetic analysis of Compound 5 binding to β-tubulin in vitro as determined by Biacore surface plasmon resonance.

FIG. 3A shows a plot of the mean tumor volume in nude mice bearing HCT116 colorectal flank tumors dosed with DM4 or Compound 5.

FIG. 3B shows the percent change in body weight of nude mice bearing HCT116 colorectal flank tumors dosed with DM4 or Compound 5 relative to day 0.

FIG. 4 depicts a Kaplan-Meier plot of nude mice bearing HCT116 colorectal flank tumors dosed with DM4 or Compound 5.

FIG. 5A depicts the ventral view and extracted lungs of nude mice inoculated with 4T1-RFP fluorescent cells via tail vein injection and imaged 11 days after inoculation and after 3 doses of vehicle or Compound 6.

FIG. 5B depicts a graph of the fluorescent signal from extracted lungs of 4T1-RFP inoculated mice after 3 doses of vehicle or Compound 6.

### DETAILED DESCRIPTION

Provided herein is a compound of Formula (I):

R²-L-R¹ (I)

or a pharmaceutically acceptable salt thereof, wherein:
R¹ is a peptide;
R² is a small molecule microtubule targeting moiety; and
L is a linker, which is covalently linked to moiety R¹ and R².

Provided herein is a compound of Formula (I):

R²-L-R¹ (I)

or a pharmaceutically acceptable salt thereof, wherein:
R¹ is a peptide having 5 to 50 amino acids;
R² is a small molecule microtubule targeting moiety; and
L is a linker, which is covalently linked to moiety R¹ and R².

Also provided herein is a compound of Formula (I):

R²-L-R¹ (I)

or a pharmaceutically acceptable salt thereof, wherein:
R¹ is a peptide capable of selectively delivering R²L- across a cell membrane having an acidic or hypoxic mantle;
R² is a small molecule microtubule targeting moiety; and
L is a linker, which is covalently linked to moiety R¹ and R².

In some embodiments, R² is a maytansine-derived microtubule targeting moiety.

Provided herein is a compound of Formula (I):

R²-L-R¹ (I)

or a pharmaceutically acceptable salt thereof, wherein:
R¹ is a peptide capable of selectively delivering R²L- across a cell membrane having an acidic or hypoxic mantle;
R² is selected from the group consisting of:

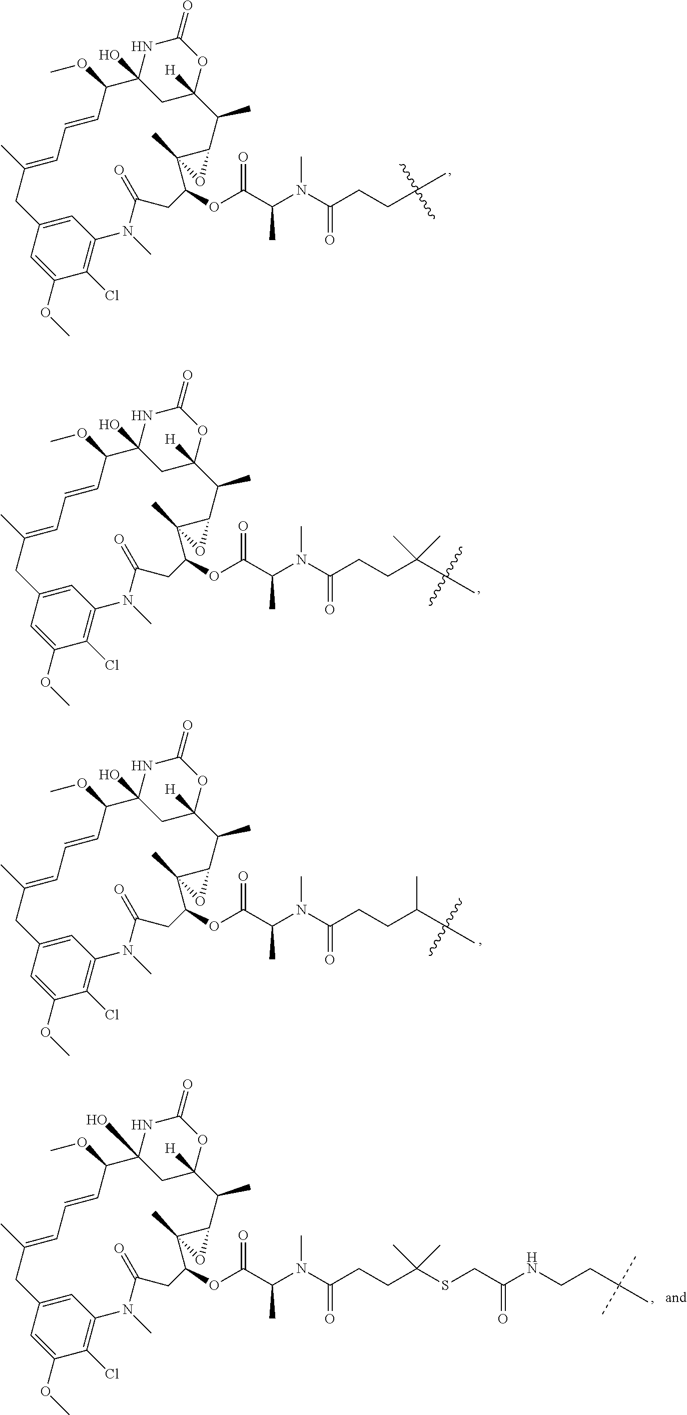

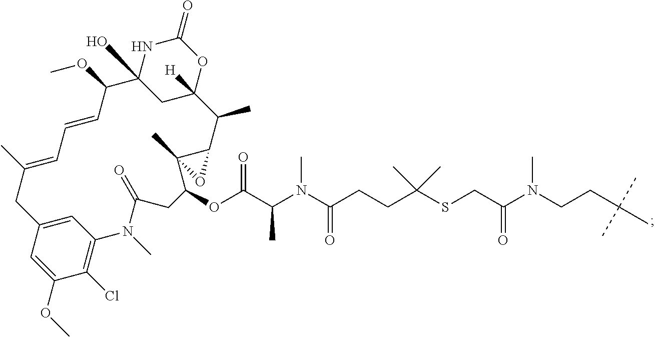

and
L is a linker, which is covalently linked to moiety R¹ and R².

Provided herein is a compound of Formula (I):

R²-L-R¹ (I)

or a pharmaceutically acceptable salt thereof, wherein:
R¹ is a peptide capable of selectively delivering R²L- across a cell membrane having an acidic or hypoxic mantle;
R² is selected from the group consisting of:

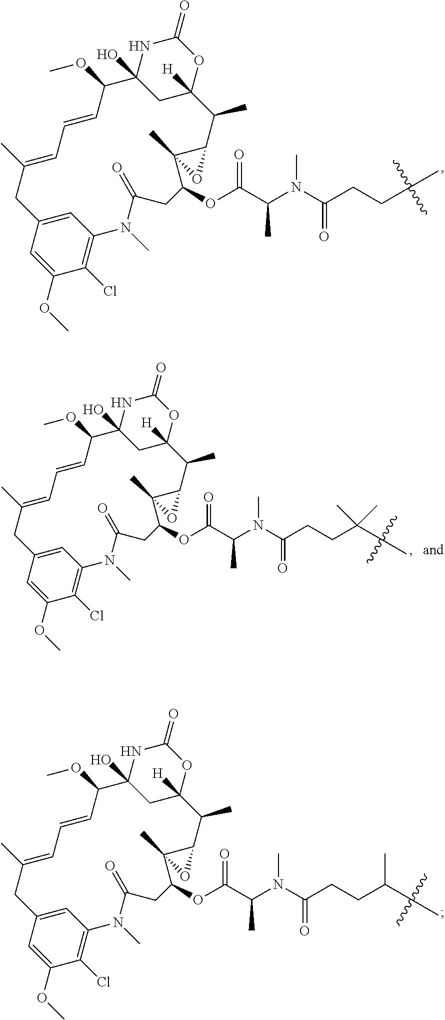

and
L is a linker, which is covalently linked to moiety R¹ and R².

Provided herein is a compound of Formula (I):

R²-L-R¹ (I)

or a pharmaceutically acceptable salt thereof, wherein:
R¹ is a peptide capable of selectively delivering R²L- across a cell membrane having an acidic or hypoxic mantle;
R² is selected from the group consisting of:

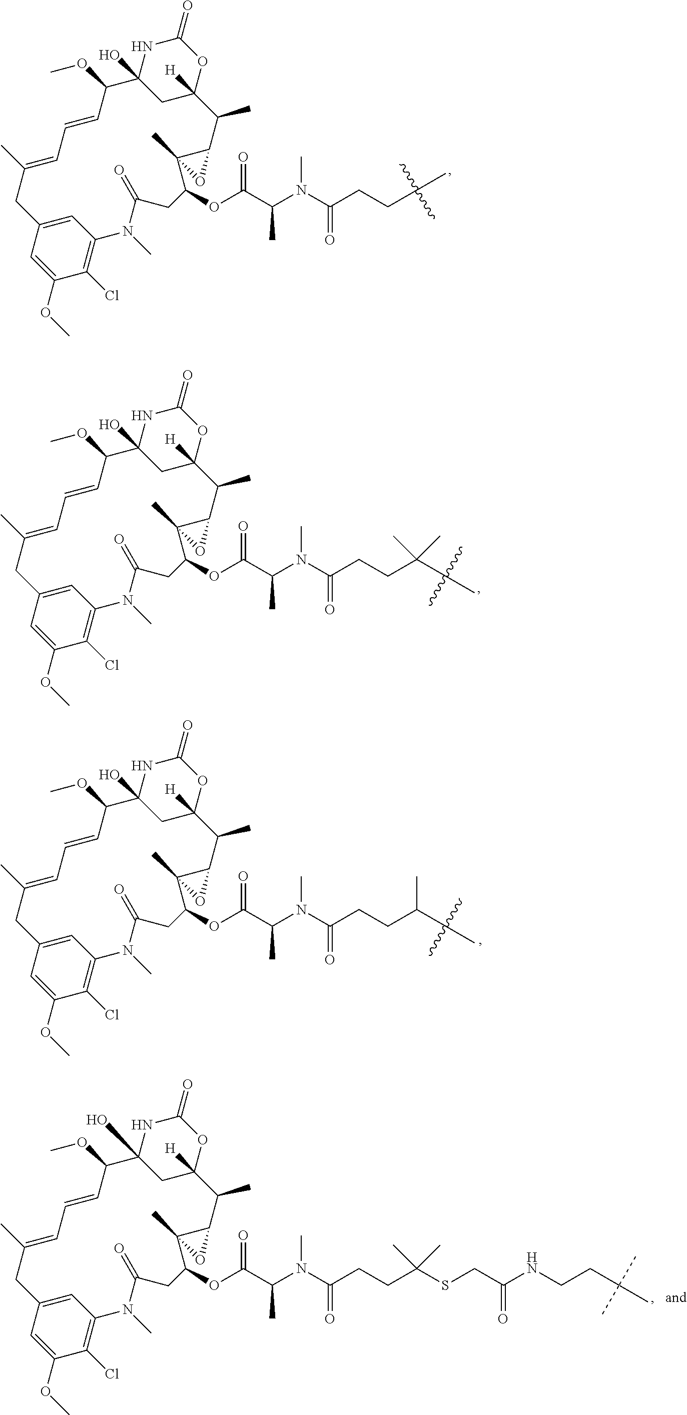

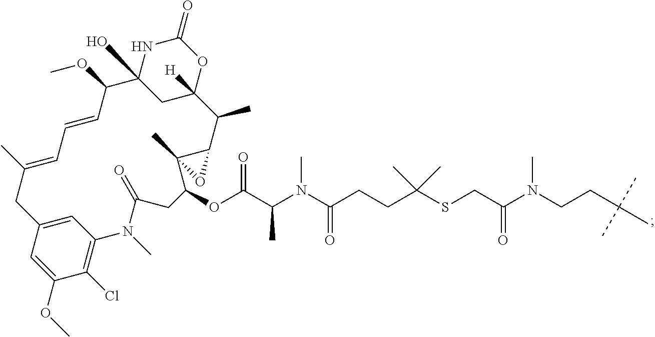

L is a group selected from:

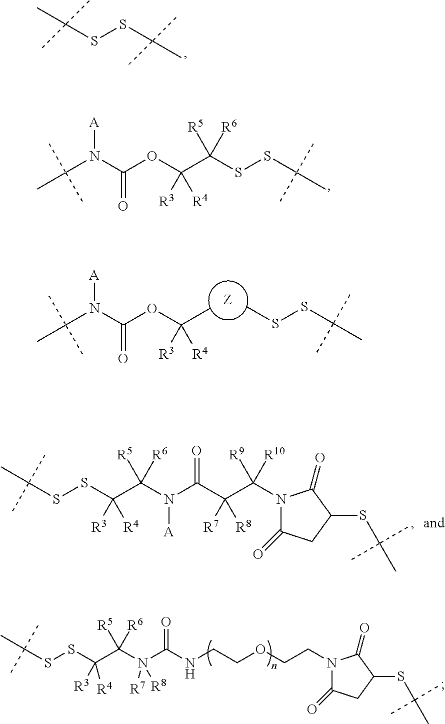

wherein
R³, R⁴, R⁵, R⁶, R⁷, R¹, R⁹, and R¹⁰ are each independently selected from H, C₁₋₄ alkyl, C₁₋₄ alkenyl, C₆₋₁₀ aryl, C₃₋₁₀ cycloalkyl, 5-10 membered heteroaryl, 4-10 membered heterocycloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1} wherein said C₁₋₄ alkyl, C₁₋₄ alkenyl, C₆₋₁₀ aryl, and 5-10 membered heteroaryl are each optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R³ and R⁴ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R³ and R⁴ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁴ and R⁶ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁵ and R⁶ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁷ and R⁸ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁷ and R⁹ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁸ and R¹⁰ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁹ and R¹⁰ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
Z is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S, wherein the C₆₋₁₀ aryl and 5-10 membered heteroaryl are each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
A is H or C₁₋₄ alkyl;
R^{a1}, R^{b1}, R^{c1}, and R^{d1} are each independently selected from H, C₁₋₆ alkyl, C₂₋₆ alkenyl, C₂₋₆ alkynyl, C₁₋₆ haloalkyl, OH, CN, NO₂, and CO₂CH₃; wherein said C₁₋₆ alkyl and C₂₋₆ alkenyl are each optionally substituted with OH, CN, NO₂, or CO₂CH; and
n is 0, 1, or 2.

Provided herein is a compound of Formula (I):

R²-L-R¹ (I)

or a pharmaceutically acceptable salt thereof, wherein:
R¹ is a peptide capable of selectively delivering R²L- across a cell membrane having an acidic or hypoxic mantle;
R² is selected from the group consisting of:

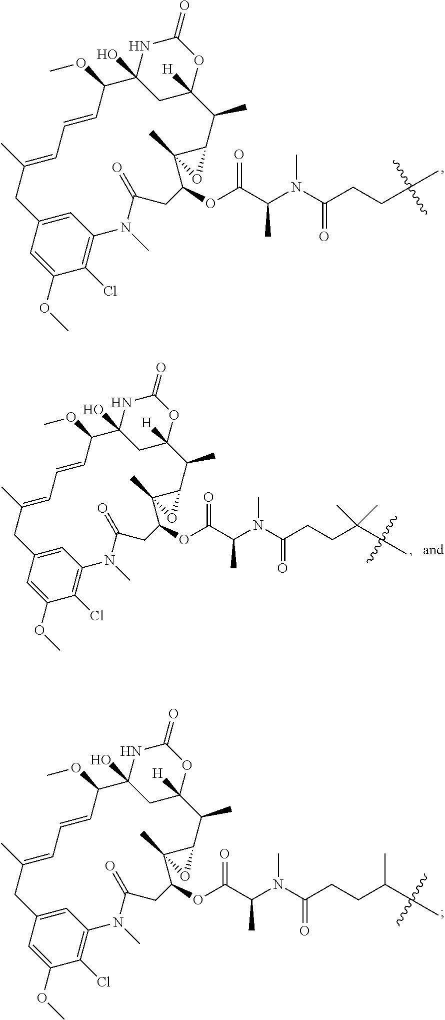

L is a group selected from:

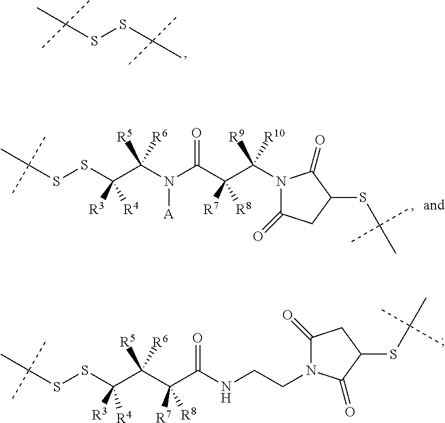

wherein
R³, R⁴, R⁵, R⁶, R⁷, R⁸, R⁹, and R¹⁰ are each independently selected from H, C₁₋₄ alkyl, C₁₋₄ alkenyl, C₆₋₁₀ aryl, 5-10 membered heteroaryl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1}, wherein said C₁₋₄ alkyl, C₁₋₄ alkenyl, C₆₋₁₀ aryl, and 5-10 membered heteroaryl are each optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R³ and R⁴ together with the carbon atom to which they are attached form an C₃₋₇ cycloalkyl group optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R³ and R⁵ together with the carbon atom to which they are attached form an C₃₋₇ cycloalkyl group optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁴ and R⁶ together with the carbon atom to which they are attached form an C₃₋₇ cycloalkyl group optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁵ and R⁶ together with the carbon atom to which they are attached form an C₃₋₇ cycloalkyl group optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁷ and R⁸ together with the carbon atom to which they are attached form an C₃₋₇ cycloalkyl group optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁷ and R⁹ together with the carbon atom to which they are attached form an C₃₋₇ cycloalkyl group optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁸ and R¹⁰ together with the carbon atom to which they are attached form an C₃₋₇ cycloalkyl group optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁹ and R¹⁰ together with the carbon atom to which they are attached form an C₃₋₇ cycloalkyl group optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
A is H or C₁₋₄ alkyl; and
R^{a1}, R^{b1}, R^{c1}, and R^{d1} are each independently selected from H, C₁₋₆ alkyl, C₂₋₆ alkenyl, C₂₋₆ alkynyl, C₁₋₆ haloalkyl, OH, CN, NO₂, and CO₂CH₃; wherein said C₁₋₆ alkyl and C₂₋₆ alkenyl are each optionally substituted with OH, CN, NO₂, or CO₂CH.

In some embodiments, the lefthand side of L attaches to R² and the righthand side of L attaches to R¹.

In some embodiments, a sulfur atom of the disulfide moiety of L is part of a cysteine residue of R¹.

As used herein, “peptide” refers to a targeting moiety comprising a 10-50 amino acid sequence, made up of naturally-occurring amino acid residues and optionally one or more non-naturally-occurring amino acids. In some embodiments, the peptide of R¹ is a peptide of 20 to 40, 20 to 30 amino acids, or 30 to 40 residues. Peptides suitable for use in the compounds of the invention are those that can insert across a cell membrane via a conformational change or a change in secondary structure in response to environmental pH changes. In this way, the peptide can target acidic tissue and selectively translocate polar, cell-impermeable molecules across cell membranes in response to low extracellular pH. In some embodiments, the peptide is capable of selectively delivering a conjugated moiety (e.g., R²L-) across a cell membrane having an acidic or hypoxic mantle having a pH less than about 6.0. In some embodiments, the peptide is capable of selectively delivering a conjugated moiety (e.g., R²L-) across a cell membrane having an acidic or hypoxic mantle having a pH less than about 6.5. In some embodiments, the peptide is capable of selectively delivering a conjugated moiety (e.g., R²L-) across a cell membrane having an acidic or hypoxic mantle having a pH less than about 5.5. In some embodiments, the peptide is capable of selectively delivering a conjugated moiety (e.g., R²L-) across a cell membrane having an acidic or hypoxic mantle having a pH between about 5.0 and about 6.0.

In certain embodiments, the peptide of R¹ includes a cysteine residue which can form the site of attachment to a payload moiety (e.g., R²L-) to be delivered across a cell membrane. In some embodiments, R¹ is attached to L through a cysteine residue of R¹. In some embodiments, the sulfur atom of the cysteine residue can form part of the disulfide bond of the disulfide bond-containing linker L.

Suitable peptides, that can conformationally change based on pH and insert across a cell membrane, are described, for example, in U.S. Pat. Nos. 8,076,451 and 9,289,508 (each of which is incorporated herein by reference in its entirety). Other suitable peptides are described, for example, in Weerakkody, et al., PNAS 110 (15), 5834-5839 (Apr. 9, 2013), which is also incorporated herein by reference in its entirety.

In some embodiments, R¹ is a peptide comprising at least one of the following sequences:

(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG;
(SEQ ID NO. 4; Pv4)
Ac-AAEQNPIYWARYADWLFTTPLLLLDLALLVDADEGTKCG;
(SEQ ID No. 5; Pv5)
AAEQNPIYWARYADWLFTTPLLLLDLALLVDADEGTC;
and
(SEQ ID No. 6; Pv6)
AAEQNPIYWWARYADWLFTTPLLLLDLALLVDADEGTCG;

wherein R¹ is attached to L through a cysteine residue of R¹.

In some embodiments, Riis a peptide comprising at least one of the following sequences:

(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG;
and
(SEQ ID No. 6; Pv6)
AAEQNPIYWWARYADWLFTTPLLLLDLALLVDADEGTCG;

wherein R¹ is attached to L through a cysteine residue of R¹.

In some embodiments, R¹ is a peptide comprising the sequence

(SEQ ID NO. 1; Pv1)
ADDQNPWRAYLDLLFPTDTLLLDLLWCG.

In some embodiments, R¹ is a peptide comprising the sequence

(SEQ ID NO. 2; Pv2)
AEQNPIYWARYADWLFTTPLLLLDLALLVDADECG.

In some embodiments, R¹ is a peptide comprising the sequence

(SEQ ID NO. 3; Pv3)
ADDQNPWRAYLDLLFPTDTLLLDLLWDADECG.

In some embodiments, R¹ is a peptide comprising the sequence

(SEQ ID NO. 4; Pv4)
Ac-AAEQNPIYWARYADWLFTTPLLLLDLALLVDADEGTKCG.

In some embodiments, R¹ is a peptide comprising the sequence

(SEQ ID NO. 5; Pv5)
AAEQNPIYWARYADWLFTTPLLLLDLALLVDADEGTC.

In some embodiments, R¹ is a peptide comprising the sequence

(SEQ ID NO. 6; Pv6)
AAEQNPIYWWARYADWLFTTPLLLLDLALLVDADEGTCG.

In some embodiments, R¹ is a peptide consisting of the sequence

(SEQ ID NO. 1; Pv1)
ADDQNPWRAYLDLLFPTDTLLLDLLWCG.

In some embodiments, R¹ is a peptide consisting of the sequence

(SEQ ID NO. 2; Pv2)
AEQNPIYWARYADWLFTTPLLLLDLALLVDADECG.

In some embodiments, R¹ is a peptide consisting of the sequence

(SEQ ID NO. 3; Pv3)
ADDQNPWRAYLDLLFPTDTLLLDLLWDADECG.

In some embodiments, R¹ is a peptide consisting of the sequence Ac-

(SEQ ID NO. 4; Pv4)
AAEQNPIYWARYADWLFTTPLLLLDLALLVDADEGTKCG.

In some embodiments, R¹ is a peptide consisting of the sequence

(SEQ ID NO. 5; Pv5)
AAEQNPIYWARYADWLFTTPLLLLDLALLVDADEGTC.

In some embodiments, R¹ is a peptide consisting of the sequence

(SEQ ID NO. 6; Pv6)
AAEQNPIYWWARYADWLFTTPLLLLDLALLVDADEGTCG.

In some embodiments, R¹ is a peptide comprising at least one sequence selected from SEQ ID NO: 7 to SEQ ID NO: 311 as shown in Table 1.

In some embodiments, R¹ is a peptide consisting of a sequence selected from SEQ ID NO: 7 to SEQ ID NO: 311 as shown in Table 1.

TABLE 1
Additional R¹ Sequences

| SEQ | |
|---|---|
| ID | |
| NO. | Sequence |
| 7 | AEQNPIYWARYADWLFTTPLLLLDLALLVDADEGT |
| 8 | GGEQNPIYWARYADWLFTTPLLLLDLALLVDADEGT |
| 9 | AEQNPIYWARYADWLFTTPLLLLDLALLVDADEGT |
| 10 | AAEQNPIYWARYADWLFTTPLLLLDLALLVDADEGTCG |
| 11 | GGEQNPIYWARYADWLFTTPLLLLDLALLVDADEGTCG |
| 12 | ACEQNPIYWARYADWLFTTPLLLLDLALLVDADEGTG |
| 13 | ACEQNPIYWARYADWLFTTPLLLLDLALLVDADEGT |
| 14 | AKEQNPIYWARYADWLFTTPLLLLDLALLVDADEGT |
| 15 | AAEQNPIYWARYADWLFTTPLLLLDLALLVDADEGTKCG |
| 16 | AKEQNPIYWARYADWLFTTPLLLLDLALLVDADECT |
| 17 | ACEQNPIYWARYANWLFTTPLLLLNLALLVDADEGTG |
| 18 | ACEQNPIYWARYAKWLFTTPLLLLKLALLVDADEGTG |
| 19 | GGEQNPIYWARYADWLFTTPLLLLDLALLVNANQGT |
| 20 | AAEQNPIYWARYADWLFTTPLLLLALALLVDADEGT |
| 21 | AAEQNPIYWARYAAWLFTTPLLLLDLALLVDADEGT |
| 22 | AAEQNPIYWARYADWLFTTALLLLDLALLVDADEGT |
| 23 | AAEQNPIYWARYADWLFTTPLLLLELALLVDADEGT |
| 24 | AAEQNPIYWARYAEWLFTTPLLLLDLALLVDADEGT |
| 25 | AAEQNPIIYWARYADWLFTDLPLLLLDLLALLVDADEGT |
| 26 | GEQNPIYWAQYADWLFTTPLLLLDLALLVDADEGTCG |
| 27 | GGEQNPIYWARYADWLFTTPLLLDLLALLVDADEGTCG |
| 28 | GGEQNPIYWARYADWLFTTPLLLLLDALLVDADEGTCG |
| 29 | GGEQNPIYWARYDAWLFTTPLLLLDLALLVDADEGTCG |
| 30 | GGEQNPIYWARYAWDLFTTPLLLLDLALLVDADEGTCG |
| 31 | AAEQNPIYWARYADWLFTTGLLLLDLALLVDADEGT |
| 32 | DDDEDNPIYWARYADWLFTTPLLLLHGALLVDADECT |
| 33 | DDDEDNPIYWARYAHWLFTTPLLLLHGALLVDADEGCT |
| 34 | DDDEDNPIYWARYAHWLFTTPLLLLHGALLVNADECT |
| 35 | DDDEDNPIYWARYAHWLFTTPLLLLHGALLVNANECT |
| 36 | AEQNPIYWARYADFLFTTPLLLLDLALLVDADET |
| 37 | AEQNPIYFARYADWLFTTPLLLLDLALLVDADEGT |
| 38 | AEQNPIYFARYADFLFTTPLLLLDLALLWDADET |
| 39 | AKEDQNPYWARYADWLFTTPLLLLDLALLVDG |
| 40 | ACEDQNPYWARYADWLFTTPLLLLDLALLVDG |
| 41 | AEDQNPYWARYADWLFTTPLLLLDLALLVDCG |
| 42 | AEDQNPYWARYADWLFTTPLLLLELALLVECG |
| 43 | AKEDQNPYWRAYADLFTPLTLLDLLALWDG |
| 44 | ACEDQNPYWRAYADLFTPLTLLDLLALWDG |
| 45 | ACDDQNPWRAYLDLLFPTDTLLLDLLW |
| 46 | TEDADVLLALDLLLLPTTFLWD |
| 47 | AEQNPIYWARYADWLFTTPL |
| 48 | AEQNPIYWARYADWLFTTPCL |
| 49 | ACEQNPIYWARYADWLFTTPL |
| 50 | AEQNPIYFARYADWLFTTPL |
| 51 | KEDQNPWARYADLLFPTTLAW |
| 52 | ACEDQNPWARYADLLFPTTLAW |
| 53 | ACEDQNPWARYADWLFPTTLLLLD |
| 54 | ACEEQNPWARYAELLFPTTLAW |
| 55 | ACEEQNPWARYAEWLFPTTLLLLE |
| 56 | ACEEQNPWARYLEWLFPTETLLLEL |
| 57 | GGEQNPIY WARYADWLFTTPLLLLDLALLV DADEGT |
| 58 | ACEQNPIY WARYADWLFTTPLLLLDLALLV |
| 59 | WARYADWLFTTPLLLLDLALLV DADEGTCG |
| 60 | WARYADWLFTTPLLLLDLALLV DADEGCT |
| 61 | GGEQNPIY WARYADWLFTTPLLLLDLALLV DADEGTCG |
| 62 | ACEQNPIY WARYADWLFTTPLLLLDLALLV DADEGT |
| 63 | AKEQNPIY WARYADWLFTTPLLLLDLALLV DADEGT |
| 64 | AKEQNPIY WARYADWLFTTPLLLLDLALLV DADEGT |
| 65 | AAEQNPIY WARYADWLFTTALLLLDLALLV DADEGT |
| 66 | ACAEQNPIY WARYADWLFTTGLLLLDLALLV DADEGT |
| 67 | AEQNPIY WARYADFLFTTALLLLDLALLV DADE_T |
| 68 | AEQNPIY FARYADWLFTTPLLLLDLALLV DADEGT |
| 69 | AEQNPIY FARYADFLFTTPLLLLDLALLW DADE_T |
| 70 | AKEDQNP_Y WARYADWLFTTPLLLLDLALLV DG____ |
| 71 | ACEDQNP_Y WARYADWLFTTPLLLLDLALLV DG____ |
| 72 | AEDQNP_Y WARYADWLFTTPLLLLDLALLV DG____ |
| 73 | AEDQNP_Y WARYADWLFTTPLLLLELALLV ECG___ |
| 74 | AKEDQNP_Y WRAYAD_LFT PLTLLDLLALW DG____ |
| 75 | ACEDQNP_Y WRAYAD_LFT PLTLLDLLALW DG____ |
| 76 | AKEDQNDP_Y WARYADWLFTTPLLLLDLALLV G_____ |
| 77 | TEDADVLLALDLLLLPTTFLWDAYRAWYPNQECA |
| 78 | GGEQNPIY WARYADWLFTTPLLLLDLALLV DADEGT |
| 79 | AEQNPIY WARYADWLFTTPL |
| 80 | AEQNPIY WARYADWLFTTPCL |
| 81 | ACEQNPIY WARYADWLFTTPL |
| 82 | ACEQNPIY FARYADWLFTTPL |
| 83 | ACDDQNP WRAYLDLLFPTDTLLLDLLW |
| 84 | ACEEQNP WRAYLELLFPTETLLLELLW |
| 85 | ACDDQNP WARYLDWLFPTDTLLLDL |
| 86 | CDNNNP WRAYLDLLFPTDTLLLDW |
| 87 | ACEEQNP WARYLEWLFPTETLLLEL |
| 88 | ACEDQNP WARYADWLFPTTLLLLD |
| 89 | ACEEQNP WARYAEWLFPTTLLLLE |
| 90 | ACEDQNP WARYADLLFPTTLAW |
| 91 | ACEDQNP WARYAELLFPTTLW |
| 92 | KEDQNP WARYADLLFPTTLW |
| 93 | DDDEDNP IYWARYAHWLFTTPLLLLHGALLVDADECT |
| 94 | DDDEDNPIYWARYAHWLFTTPLLLLDGALLVDADECT |
| 95 | DDDEDNPIYWARYAHWLFTTPLLLLHGALLVNADECT |
| 96 | DDDEDNPIYWARYAFIWLFTTPLLLLHGALLVNANECT |
| 97 | DDDEDNPIYWARYADWLFTTPLLLLHGALLVDADECT |
| 98 | ACEQNPIYWARYADWLFTTPLLLLDLALLVDADEGIG |
| 99 | ACEQNPIYWARYADWLFTTPLLLLDLALLVDADET |
| 100 | ACEQNPIYWARYADWLFTTPLLLLDLALLVDADEGT |
| 101 | GGEQNPIYWARYADWLFTTPLLLDLLALLVDADEGTCG |
| 102 | GGEQNPIYWARYADWLFTTPLLLLLDALLVDADEGTCG |
| 103 | GGEQNPIYWARYAWDLFTTPLLLLDLALLVDADEGTCG |
| 104 | AAEQNPIYWARYAEWLFTTPLLLLDLALLVDADEGTCG |
| 105 | AAEQNPIYWARYAEWLFTTPLLLLELALLVDADEGTCG |
| 106 | GGEQNPIYWARYDAWLFTTPLLLLDLALLVDADEGTCG |
| 107 | GGEQNPIYWAQYDAWLFTTPLLLLDLALLVDADEGTCG |
| 108 | GGEQNPIYWAQDYAWLFTTPLLLLDLALLVDADEGTCG |
| 109 | AAEQNPIYWARYAAWLFTTPLLLLDLALLVDADEGTCG |
| 110 | ACEQNPIYWARYANWLFTTPLLLLNLALLVDADEGTG |
| 111 | DDDEDNPIYWARYAFIWLFTTPLLLLHGALLVNANECT |
| 112 | DDDEDNPIYWARYAHWLFTTPLLLLHGALLVNADECT |
| 113 | DDDEDNPIYWARYADWLFTTPLLLLHGALLVDADECT |
| 114 | DDDEDNPIYWARYAHWLFTTPLLLLHGALLVDADECT |
| 115 | DDDEDNPIYWARYAHWLFTTPLLLLDGALLVDADECT |
| 116 | GGEQNPIYWARYADWLFTTPLLLLDLALLVNANQGT |
| 117 | AAEQNPIYWARYADWLFTTPLLLLELALLVDADEGTCG |
| 118 | AAEQNPIYWARYAEWLFTTPLLLLELALLVDADEGTCG |
| 119 | AAEQNPIYWARYADWLFTTPLLLLELALLVDADEGTKCG |
| 120 | GGEQNPIYWAQYADWLFTTPLLLLDLALLVDADEGTCG |
| 121 | GGEQNPIYWAQYDAWLFTTPLLLLDLALLVDADEGTCG |
| 122 | GGEQNPIYWAQDYAWLFTTPLLLLDLALLVDADEGTCG |
| 123 | GGEQNPIYWARYADWLFTTPLLLLDALLVNANQGT |
| 124 | DDDEDNPIYWARYAHWLFTTPLLLLHGALLVNADECT |
| 125 | DDDEDNPIYWARYAHWLFTTPLLLLHGALLVNANECT |
| 126 | ACEQNPIYWARYAKWLFTTPLLLLKLALLVDADEGTG |
| 127 | GGEQNPIYWAQDYAWLFTTPLLLLDLALLVDADEGTCG |
| 128 | GGEQNPIYWAQYDAWLFTTPLLLLDLALLVDADEGTCG |
| 129 | GGEQNPIYWAQYADWLFTTPLLLLDLALLVDADEGTCG |
| 130 | AAEQNPIYWARYAAWLFTTPLLLLDLALLVDADEGTCG |
| 131 | AAEQNPIYWARYADWLFTDLPLLLLDLLALLVDADEGT |
| 132 | GGEQNPIYWARYADWLFTTPLLLLLDALLVDADEGTCG |
| 133 | GGEQNPIYWARYADWLFTTPLLLDLLALLVDADEGTCG |
| 134 | AAEQNPIYWARYADWLFTTGLLLLDLALLVDADEGT |
| 135 | AEQNPIYWARYAAWLFTTPLLLLDLALLVDADEGTCG |
| 136 | GGEQNPIYWAQYDAWLFTTPLLLLDLALLVDADEGTCG |
| 137 | GGEQNPIYWAQDYAWLFTTPLLLLDLALLDADEGTCG |
| 138 | GGEQNPIYWARYDAWLFTTPLLLLDLALLVDADEGTCG |
| 139 | AAEQNPIYWARYADWLFTTPLLLLALALLVDADEGTCG |
| 140 | AAEQNPIYWARYADWLFTTPLLLLDLALLVDADEGTKC |
| | G . . . EGTK(rhodamine)C(phalloidin)G |
| 141 | AAEQNPIYWARYADWLFTTPLLLLELALLDADEGTKCG |
| 142 | AAEQNPIYWARYADWLFTTPLLLLDLALLVDADEGTCG |
| 143 | AAEQNPIYWARYADWLFTTPLLLLDLALLVDADEGTC |
| | (phalloidin)G |
| 144 | GGEQNPIYWARYADWLFTTPLLLLDLALLVDADEGTCG |
| 145 | ACEQNPIYWARYADWLFTTPLLLLDLALLVDADET |
| 146 | ACEQNPIYWARYADWLFTTPLLLLDLALLVDADEGTG |
| 147 | ACEQNPIYWARYADWLFTTPLLLLDLALLVDADEGT |
| 148 | GGEQNPIYWARYADWLFTTPLLLLDLALLVNANQGT |
| 149 | DDDEDNPIYWARYAHWLFTTPLLLLHGALLVNADECT |
| 150 | DDDEDNPIYWARYAHWLFTTPLLLLHGALLVNANECT |
| 151 | GGEQNPIYWARYADWLFTTPLLLLDLALLVDADEGTCG |
| 152 | AAEQNPIYWARYADWLFTTPLLLLDLALLVDADEGTC |
| | (phalloidin)G |
| 153 | AAEQNPIYWARYADWLFTTPLLLLELALLVDADEGTKCG |
| 154 | AAEQNPIYWARYADWLFTTPLLLLDLALLVDADEGTKCG |
| 155 | DDDEDNPIYWARYAHWLFTTPLLLLBGALLVDADECT |
| 156 | DDDEDNPIYWARYAHWLFTTPLLLLDGALLVDADECT |
| 157 | DDDEDNPIYWARYAHWLFTTPLLLLBGALLVNADECT |
| 158 | DDDEDNPIYWARYAHWLFTTPLLLLBGALLVNANECT |
| 159 | DDDEDNPIYWARYADWLFTTPLLLLIBGALLVDADECT |
| 160 | DDDEDNPIYWARYADWTFTTPLLLLHGALLVDADECT |
| 161 | DDDEDNPIYWARYAHWLFTTPLLLLDGALLVDADECT |
| 162 | DDDEDNPIYWARYAHWLFTTPLLLLHGALLVDADECT |
| 163 | DDDEDNPIYWARYAHWLFTTPLLLLHGALLVNADECT |
| 164 | DDDEDNPIYWARYHWLFTTPLLLLHGALLVNANECT |
| 165 | DDDEDNPIYWARYAHWLFTTPLLLLHGALLVNANECT |
| 166 | DDDEDNPIYWARYAHWLFTTPLLLLHGALLVNADECT |
| 167 | DDDEDNPIYWARYADWLFTTPLLLLHGALLVDADECT |
| 168 | DDDEDNPIYWARYAHWLFTTPLLLLHGALLVDADECT |
| 169 | DDDEDNPIYWARYAHWLFTTPLLLLDGALLVDADECT |
| 170 | GGEQNPIYWARYADWLFTTPLLLLDLALLVNANQGT |
| 171 | DDDEDNPIYWARYAHWLFTTPLLLLHGALLVNADECT |
| 172 | DDDEDNPIYWARYADWLFTTPLLLLHGALLVDADECT |
| 173 | DDDEDNPIYWARYAHWLFTTPLLLLHGALLVDADECT |
| 174 | DDDEDNPIYWARYAHMLFTTPLLLLDGALLVDADECT |
| 175 | DDDEDNPIYWARYAHWLFTTPLLLLHGALLVNANECT |
| 176 | DDDEDNPIYWARYAHWLFTTPLLLLDGALLVDADECT |
| 177 | DDDEDNPIYWARYADWLFTTPLLLLHGALLVDADECT |
| 178 | DDDEDNPIYWARYAHWLFTTPLLLLHGALLVDADECT |
| 179 | DDDEDNPIYWARYAHWLFTTPLLLLHGALLVNADECT |
| 180 | DDDEDNPIYWARYAHWLFTTPLLLLHGALLVNANECT |
| 181 | AAEQNPIYWARYADWLFTTGLLLLDLALLVDADEGT |
| 182 | GGEQNPIYWARYAWDLFTTPLLLLDLALLVDADEGTCG |
| 183 | GGEQNPIYWARYDAWLFTTPLLLLDLALLVDADEGTCG |
| 184 | GGEQNPIYWAQYDAWLFTTPLLLLDLALLVDADEGTCG |
| 185 | GGEQNPIYWAQDYAWLFTTPLLLLDLALLVDADEGTCG |
| 186 | AAEQNPIYWARYAAWLFTTPLLLLDLALLVDADEGTCG |
| 187 | GGEQNPIYWARYADWLFTTPLLLLDALLVDADEGTCG |
| 188 | GGEQNPIYWARYADWLFTTPLLLDLLALLVDADEGTCG |
| 189 | GGEQNPIYWARYADWLFTTPLLLDLLALLVDADEGTCG |
| 190 | GGEQNPIYWARYADWLFTTPLLLLLDALLVDADEGTCG |
| 191 | GGEQNPIYWAQYADWLFTTPLLLLDLALLVDADEGTCG |
| 192 | GGEQNPIYWAQYDAWLFTTPLLLLDLALLVDADEGTCG |
| 193 | GGEQNP1YWAQDYAWLFTTPLLLLDLALLVDADEGTCG |
| 194 | GGEQNPIYWAQYDAWLFTTPLLLLDLALLVDADEGTCG |
| 195 | GGEQNPIYWAQDYAWLFTTPLLLLDLALLVDADEGTCG |
| 196 | GGEQNPIYWAQYADWLFTTPLLLLDLALLVDADEGTCG |
| 197 | AAEQNPIYWARYAAWLFTTPLLLLDLALLVDADEGTCG |
| 198 | GGEQNPIYWAQDYAWLFTTPLLLLDLALLVDADEGTCG |
| 199 | GGEQNPIYWAQYDAWLFTTPLLLLDLALLVDADEGTCG |
| 200 | GGEQNPIYWAQYADWLFTTPLLLLDLALLVDADEGTCG |
| 201 | AAEQNPIYWARYAAWLFTTPLLLLDLALLVDADEGTCG |
| 202 | AAEQNPIYWARYADWLFTTPLLLLELALLVDADEGTKCG |
| 203 | . . . EGTK(rhidamine)C(phalloidin)G |
| 204 | AAEQNPIYWARYADWLFTTPLLLLDLALLVDADEGTKCG |
| 205 | ACEQNPIYWARYADWLFTTPLLLLDLALLVDADEGTG |
| 206 | AAEQNPIYWARYADWLFTTPLLLLDLALLVDADEGTC |
| | (phalloidin)G |
| 207 | AAEQNPIYWARYADWLFTTPLLLLDLALLVDADEGTKCG |
| 208 | AAEQNPIYWARYADWLFTTPLLLLELALLVDADEGTKCG |
| 209 | AAEQNPIYWARYADWLFTDLPLLLLDLLALLVDADEGT |
| 210 | AAEQNPIYWARYAAWLFTTPLLLLDLALLVDADEGTCG |
| 211 | GGEQNPIYWAQYDAWLFTTPLLLLDLALLVDADEGTCG |
| 212 | GGEQNPIYWAQDYAWLFTTPLLLLDLALLVDADEGTCG |
| 213 | GGEQNPIYWARYDAWLFTTPLLLLDLALLVDADEGTCG |
| 214 | AAEQNPIYWARYAEWLFTTPLLLLDLALLVDADEGTCG |
| 215 | AAEQNPIYWARYAEWLFTTPLLLLELALLVDADEGTCG |
| 216 | AAEQNPIYWARYADWLFTTPLLLLALALLVDADEGTCG |
| 217 | AAEQNPIYWARYADWLFTTPLLLLELALLVDADEGTCG |
| 218 | AAEQNPIYWARYAEWLFTTPLLLLELALLVDADEGTCG |
| 219 | AAEQNPIYWARYADWLFTTPLLLLELALLVDADEGTKCG |
| 220 | ACEQNPIYWARYAKWLFTTPLLLLKLALLVDADEGTG |
| 221 | ACEQNPIYWARYANWLFTTPLLLLNLALLVDADEGTG |
| 222 | AAEQNPIYWARYADWLFTTALLLLDLALLVDADEGT |
| 223 | AEQNPIYFARYADLLFPTTLAW |
| 224 | AEQNPIYWARYADLLFPTTLAF |
| 225 | AEQNPIYWARYADLLFPTTLAW |
| 226 | ACEQNPIYWARYADWLFTTPLLLLDLALLVDADET |
| 227 | GGEQNPIYWARYADWLFTTPLLLLDLALLVDADEGT |
| 228 | AAEQNPIYWARYADWLFTTPLLLLDLALLVDADEGTCG |
| 229 | AAEQNPIYWARYADWLFTTPLLLLDLALLVDADEGTKCG |
| 230 | AKEQNPIYWARYADWLFTTPLLLLDLALLVDADECT |
| 231 | CCTCTTACCTCAGTTACA |
| 232 | D-Arg8D-Arg8-CCTCTTACCTCAGTTACA |
| 233 | D-Lys4D-Lys4-CCTCTTACCTCAGTTACA |
| 234 | S-S-CCTCTTACCTCAGTTACA |
| 235 | S-S-CCTCTGACCTCATTTACA |
| 236 | D-Arg8-DecaD-Arg8-Deca-CCTCTTACCTCAGTTACA |
| 237 | D-Arg8-Deca-mismatchD-Arg8-Deca- |
| | CCTCTGACCTCATTTACA |
| 238 | S-S-CCTCTTACCTCAGTTACA |
| 239 | AAEQNPIYWARYADWLFTTPLLLLDLALLVDADEGTCG |
| 240 | AEDQNPYWARYDWLFTTPLLLLDLALLVDCG |
| 241 | AEDQNPYWARYADWLFTTPLLLLELALLVECG |
| 242 | AEQNPIYWARYADWLFTTPLLLLDLALLVDADEGCT |
| 243 | ACEQNPIYWARYADWLFTTPLLLLDLALLVDADET |
| 244 | AE-QN-PIYWARYADWLFTTPLLLLDLALLVDADEGT- |
| | COOH |
| 245 | AEDQN-P-YWARYADWLFTTPLLLLDLALLVD---G-- |
| | COOH |
| 246 | AEDQNDP-YWARYADWLFTTPLLLLDLALLV----G-- |
| | COOH |
| 247 | AEQNPIYWARYADFLFTTPLLLLDLALLV DADET-COOH |
| 248 | AEQNPI YFARYADWLFTTPLLLLDLALLV DADET- |
| | COOH |
| 249 | AEQNPI YFARYADFLFTTPLLLLDLALLW DADET- |
| | COOH |
| 250 | AE-QN-PI YWARYADWLFTTPLLLLDLALLV DADEGCT- |
| | COOH |
| 251 | AEDQN-PI YWARYADWLFTTPLLLLDLALLV DC--G-T- |
| | COOH |
| 252 | AEDQNDPI YWARYADWLFTTPLLLLELALLV EC--G-T- |
| | COOH |
| 253 | Chelate-ACEEQNPWARYLEWLFPTETLLLEL |
| 254 | AEQNPIY WARYADWLFTTPLLLLDLALLV DADEGT- |
| | COOH |
| 255 | AKEDQNPY WARYADWLFTTPLLLLDLALLV DG-COOH |
| 256 | AKEDQNDPY WARYADWLFTTPLLLLDLALLV G-COOH |
| 257 | AEQNPI YWARYADWLFTTPLLLLDLALLV DADEGC- |
| | Biotin-T-COO H |
| 258 | AEDQNP YWARYADWLFTTPLLLLDLALLV DC-Biotin- |
| | G-COOH |
| 259 | AEDQNP YWARYADWLFTTPLLLLELALLV EC-Biotin- |
| | G-COOH |
| 260 | ACEQNPIY WARYADWLFTTPLLLLDLALLV DADEGT |
| 261 | ACEDQNPY WARYADWLFTTPLLLLDLALLV DG |
| 262 | ACEDQNPY WRAYADLFTPLTLLDLLALW DG |
| 263 | ACDDQNP WRAYLDLLFPTDTLLLDLLW |
| 264 | WRAYLELLFPTETLLLELLW |
| 265 | WARYLDWLFPTDTLLLDL |
| 266 | WRAYLDLLFPTDTLLLDW |
| 267 | WARYLEWLFPTETLLLEL |
| 268 | WAQYLELLFPTETLLLEW |
| 269 | WRAYLELLFPTETLLLEW |
| 270 | WARYADWLFPTTLLLLD |
| 271 | WARYAEWLFPTTLLLLE |
| 272 | ACEDQNP WARYADLLFPTTLAW |
| 273 | ACEEQNP WARYAELLFPTTLAW |
| 274 | Ac-TEDADVLLALDLLLLPTTFLWDAYRAWYPNQECA-Am |
| 275 | CDDDDDNPNY WARYANWLFTTPLLLLNGALLV EAEET |
| 276 | CDDDDDNPNY WARYAPWLFTTPLLLLPGALLV EAEET |
| 277 | Ac-AEQNPIYWARYADWLFTTPLLLLDLALLVDADEGCT |
| 278 | Ac-AKEQNPIYWARYADWLFTTPLLLLDLALLVDADEGTG |
| 279 | ACEQNPIYWARYANWLFTTPLLLLNLALLVDADEGT |
| 280 | Ac-AAEQNPIYWARYADWLFTTPLLLLELALLVDADEGTK |
| | CG |
| 281 | DDDEDNPIYWARYADWLFTTPLLLLHGALLVDADET |
| 282 | CDDDEDNPIYWARYAHWLFTTPLLLLHGALLVDADET |
| 283 | DDDEDNPIYWARYAHWLFTTPLLLLHGALLVDADEGT |
| 284 | DDDEDNPIYWARYAHWLFTTPLLLLHGALLVNADECT |
| 285 | DDDEDNPIYWARYAHWLFTTPLLLLHGALLVNANEGT |
| 286 | AKEDQNDPYWARYADWLFTTPLLLLDLALLVG |
| 287 | AEDQNPYWARYADWLFTTPLLLLELALLVCG |
| 288 | AKDDQNPWRAYLDLLFPTDTLLLDLLWC |
| 289 | ACEEQNPWRAYLELLFPTETLLLELLW |
| 290 | ACDDQNPWARYLDWLFPTDTLLLDL |
| 291 | CDNNNPWRAYLDLLFPTDTLLLDW |
| 292 | CEEQQPWAQYLELLFPTETLLLEW |
| 293 | EEQQPWRAYLELLFPTETLLLEW |
| 294 | CDDDDDNPNYWARYANWLFTTPLLLLNGALLVEAEET |
| 295 | CDDDDDNPNYWARYAPWLFTTPLLLLPGALLVEAEE |
| 296 | AEQNPIYFARYADLLFPTTLAW |
| 297 | AEQNPIYWARYADLLFPTTLAF |
| 298 | AEQNPIYWARYADLLFPTTLAW |
| 299 | KEDQNPWARYADLLFPTTLW |
| 300 | ACEEQNPQAEYAEWLFPTTLLLLE |
| 301 | AAEEQNPWARYLEWLFPTETLLLEL |
| 302 | AKEEQNPWARYLEWLFPTETLLLEL |
| 303 | AAEQNPIYWARYADWLFTTPLLLLDLALLVDADEGTGG |
| 304 | XXEXNPIYWAXXXXXLFTXXLLLXXXALLVXAXXXTXG |
| 305 | DAAEQNPIYWARYADWLFTTLPLLLLDLLALLVDADEGTK |
| | GG |
| 306 | GGEQNPIYWARYADWLFTTPLLLLDLALLVDADEGTGG |
| 307 | XXEXNPIYWAXXXXXLFTXXLLLXXXALLVXAXXXTGG |
| 308 | DGGEQNDPIYWARYADWLFTTLPLLLLDLLALLVDA |
| | DEGCTXGG |
| 309 | AAEQNPIYWARYADWLFTTPLLLLDLALLVDADEGTCG |
| 310 | AEDQNPYWARYDWLFTTPLLLLDLALLVDCG |
| 311 | GLAGLAGLLGLEGLLGLPLGLLEGLWLGLELEGN |

Any of the recited peptides useful in the present invention can be modified to include a cysteine residue by replacing a non-cysteine residue with cysteine, or appending a cysteine residue to either the N-terminus or C-terminus.

In some embodiments, the peptide of R¹ is a conformationally restricted peptide. A conformationally restricted peptide can include, for example, macrocyclic peptides and stapled peptides. A stapled peptide is a peptide constrained by a covalent linkage between two amino acid side-chains, forming a peptide macrocycle. Conformationally restricted peptides are described, for example, in Guerlavais et al., Annual Reports in Medicinal Chemistry 2014, 49, 331-345; Chang et al., Proceedings of the National Academy of Sciences of the United States of America (2013), 110(36), E3445-E3454; Tesauro et al., Molecules 2019, 24, 351-377; Dougherty et al., Journal of Medicinal Chemistry (2019), 62(22), 10098-10107; and Dougherty et al., Chemical Reviews (2019), 119(17), 10241-10287, each of which is incorporated herein by reference in its entirety.

In some embodiments, R¹ is a peptide having 10 to 50 amino acids. In some embodiments, R¹ is a peptide having 20 to 40 amino acids. In some embodiments, R¹ is a peptide having 20 to 40 amino acids. In some embodiments, R¹ is a peptide having 10 to 20 amino acids. In some embodiments, R¹ is a peptide having 20 to 30 amino acids. In some embodiments, R¹ is a peptide having 30 to 40 amino acids.

Suitable small molecule microtubule targeting moieties (e.g., R²) can be cytotoxic compounds like maytansinoids that may have undesirable side effects when delivered systemically because of their possible deleterious effect on normal tissue. Small molecule microtubule targeting agents include, but are not limited to, maytansinoids, aclitaxel, docetaxel, epothilones, discodermolide, the vinca alkaloids, colchicine, combretastatins, and derivatives and analogues of the aforementioned. Microtubule targeting agents are described in Tangutur, A. D., Current Topics in Medicinal Chemistry, 2017 17(22): 2523-2537. Microtubule-targeting agents also include maytansinoids, such as maytansine (DM1) and derivatives and analogues thereof, which are described in Lopus, M, Cancer Lett., 2011, 307(2): 113-118; and Widdison, W., J. Med. Chem. 2006, 49:4392-4408.

In some embodiments, R² is the following group:

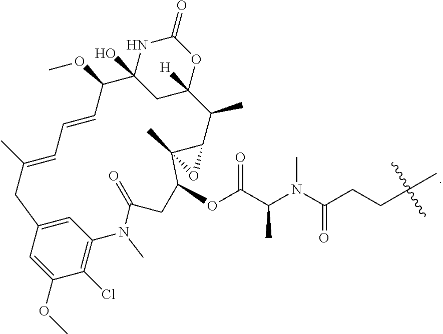

In some embodiments, R² is the following group:

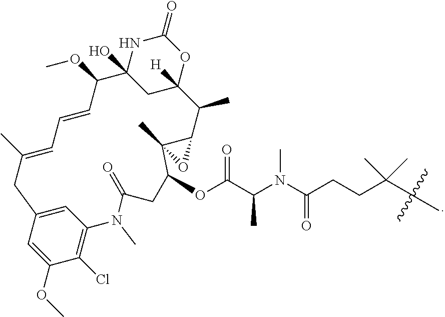

In some embodiments, R² is the following group:

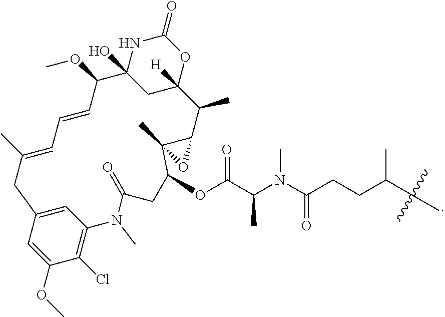

In some embodiments, R² is the following group:

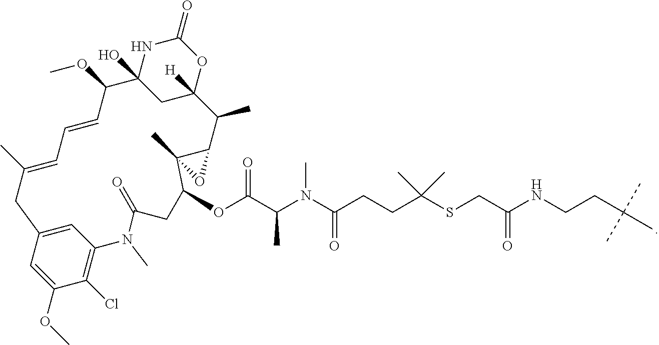

In some embodiments, R² is the following group:

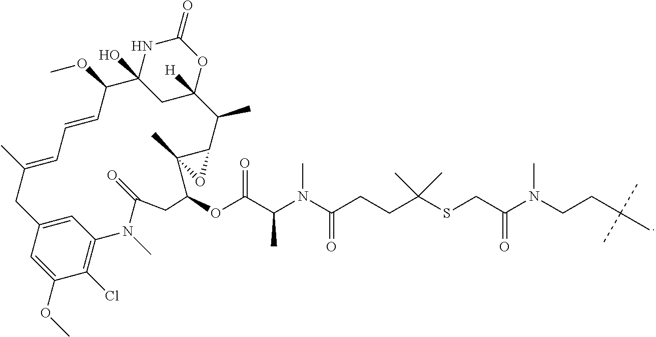

In some embodiments, R² is a maytansinoid. In some embodiments, R² is DM1 or DM4. In some embodiments, R² is DM1. In some embodiments, R² is DM4.

In some embodiments, L is a linking moiety that covalently connects R¹ and R², and functions to release a moiety containing R² in the vicinity of acidic or hypoxic tissue, such as inside a cell of diseased tissue.

In some embodiments, L is a linking chain of 1 to 40, 1 to 30, 1 to 25, 1 to 20, 1 to 15, 1 to 10, or 1 to 5 chain atoms (including both carbon and heteroatoms), which is optionally substituted with 1-10 R^{q} substituents, and wherein one or more chain carbon atoms of L can be oxidized to form a carbonyl (C═O), and wherein one or more N and S chain atoms can each be optionally oxidized to form an amine oxide, sulfoxide or sulfonyl group; wherein
each R^{q} is independently selected from OH, CN, —COOH, NH₂, halo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl, C₃₋₆ cycloalkyl, NH(C₁₋₆ alkyl) and N(C₁₋₆ alkyl)₂, wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5- or 6-membered heteroaryl or 4-6 membered heterocycloalkyl; and
two R^{q} groups together with the chain atoms to which they are attached can form a phenyl, 5-6 membered heteroaryl, 4-6 membered heterocycloalkyl, or C₃₋₆ cycloalkyl ring.

In some embodiments, R^{q} is independently selected from OH, CN, —COOH, NH₂, halo, C₁₋₆ haloalkyl, C₁₋₆ alkyl, C₁₋₆ alkoxy, C₁₋₆ haloalkoxy, NH(C₁₋₆ alkyl) and N(C₁₋₆ alkyl)₂.

In some embodiments, L is the following group:

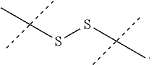

In some embodiments, L is the following group:

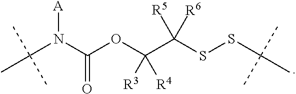

In some embodiments, L is the following group:

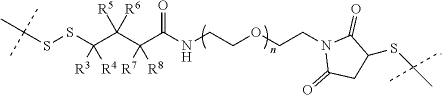

In some embodiments, n is 0. In some embodiments, n is 1. In some embodiments, n is 2.

In some embodiments, L is the following group:

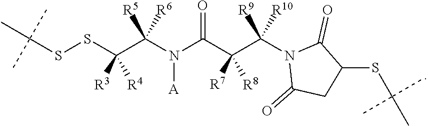

In some embodiments, L is the following group:

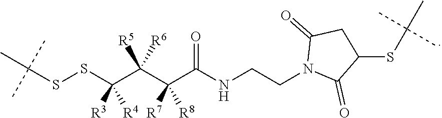

In some embodiments, L is the following group:

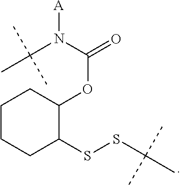

In some embodiments, L is the following group:

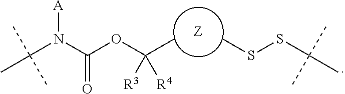

In some embodiments, R³, R⁴, R⁵, R⁶, R⁷, R⁸, R⁹, and R¹⁰ are each independently selected from H and C₁₋₄ alkyl. In some embodiments, R³, R⁴, R⁵, R⁶, R⁷, R⁸, R⁹, and R¹⁰ are each H.

In some embodiments, R³ and R⁴ are each independently selected from H and C₁₋₄ alkyl. In some embodiments, R³ and R⁴ are each H.

In some embodiments, R⁵ and R⁶ are each independently selected from H and C₁₋₄ alkyl. In some embodiments, R⁵ and R⁶ are each H.

In some embodiments, R⁷ and R⁸ are each independently selected from H and C₁₋₄ alkyl. In some embodiments, R⁷ and R⁸ are each H.

In some embodiments, R⁹ and R¹⁰ are each independently selected from H and C₁₋₄ alkyl. In some embodiments, R⁹ and R¹⁰ are each H.

In some embodiments, A is H. In some embodiments, A is C₁₋₄ alkyl. In some embodiments, A is CH₃.

In some embodiments, Z is C₆₋₁₀ aryl, optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1}.

In some embodiments, Z is phenyl, optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1}.

In some embodiments, Z is phenyl.

In some embodiments, the compound of the invention is a compound of Formula (II):

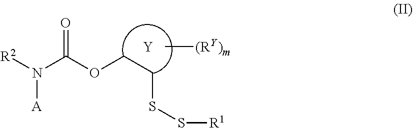

or a pharmaceutically acceptable salt thereof, wherein.
R¹ is a peptide;
R² is a small molecule microtubule targeting moiety;
A is H or C₁₋₄ alkyl;
Ring Y is a monocyclic C₅₋₇ cycloalkyl ring or a monocyclic 5-7 membered heterocycloalkyl ring;
each R^{Y} is independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or two adjacent R^{Y} together with the atoms to which they are attached form a fused monocyclic C₅₋₇ cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
R^{a1}, R^{b1}, R^{c1}, and R^{d1} are each independently selected from H, C₁₋₄ alkyl, C₂₋₄ alkenyl, C₂₋₄ alkynyl, each optionally substituted with 1, 2, or 3 substituents independently selected from halo, OH, CN, and NO₂; and
m is 0, 1, 2, or 3.

In some embodiments of compounds of Formula (II), R¹ is a peptide comprising the sequence of SEQ ID NO: 1, SEQ ID NO:2, SEQ ID NO:3, SEQ ID NO:4, or SEQ ID NO:5.

In some embodiments of compounds of Formula (II), R¹ is Pv1, Pv2, Pv3, Pv4, or Pv5.

In some embodiments of compounds of Formula (II), R¹ is attached to the core via a cysteine residue of R¹ wherein one of the sulfur atoms of the disulfide moiety in Formula II is derived from the cysteine residue.

In some embodiments of compounds of Formula (II), R² is a maytansinoid. In some embodiments of Formula (II), R² is DM1 or DM4. In some embodiments of Formula (II), R² is DM1. In some embodiments of Formula (II), R² is DM4.

In some embodiments of compounds of Formula (II), R² is the following group:

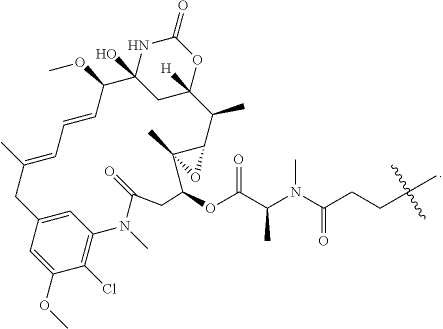

In some embodiments of compounds of Formula (II), R² is the following group:

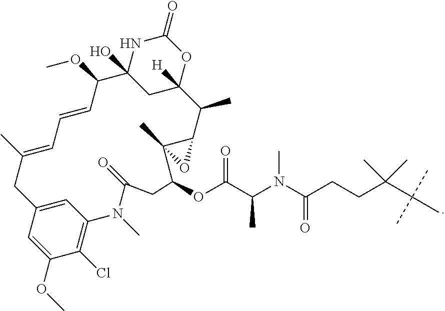

In some embodiments of compounds of Formula (II), R² is the following group:

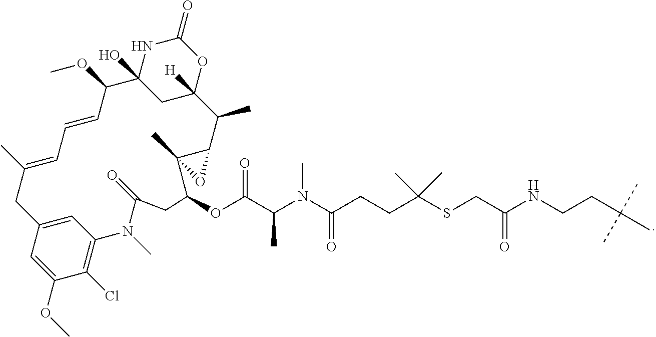

In some embodiments of compounds of Formula (II), R² is the following group:

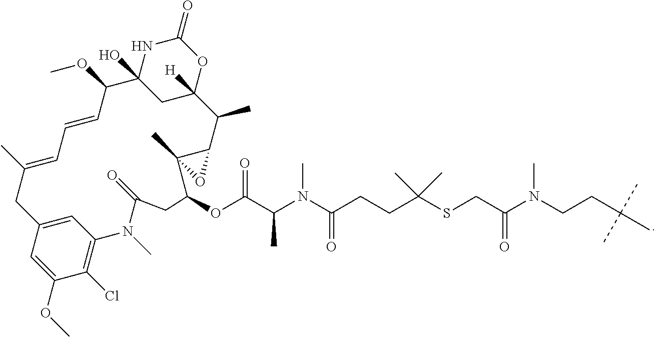

In some embodiments of compounds of Formula (II), A is H. In some embodiments of compounds of Formula (II), A is C₁₋₄ alkyl. In some embodiments of compounds of Formula (II), A is CH₃.

In some embodiments of compounds of Formula (II), Ring Y is a monocyclic C₅₋₇ cycloalkyl ring.

In some embodiments of compounds of Formula (II), Ring Y is a cyclopentyl ring.

In some embodiments of compounds of Formula (II), Ring Y is a cyclohexyl ring.

In some embodiments of compounds of Formula (II), Ring Y is a cycloheptyl ring.

In some embodiments of compounds of Formula (II), Ring Y is a monocyclic 5-7 membered heterocycloalkyl ring.

In some embodiments of compounds of Formula (II), Ring Y is a 5-membered heterocycloalkyl ring.

In some embodiments of compounds of Formula (II), Ring Y is a 6-membered heterocycloalkyl ring.

In some embodiments of compounds of Formula (II), Ring Y is a 7-membered heterocycloalkyl ring.

In some embodiments of compounds of Formula (II), two adjacent R^{Y} together with the atoms to which they are attached form a fused monocyclic C₅₋₇ cycloalkyl ring, a fused monocyclic 5-7 membered heterocycloalkyl ring, a fused C₆₋₁₀ aryl ring, or a fused 6-10 membered heteroaryl ring, each of which is optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1}.

In some embodiments of compounds of Formula (II), m is 0.

In some embodiments of compounds of Formula (II), m is 1.

In some embodiments of compounds of Formula (II), m is 2.

In some embodiments of compounds of Formula (II), m is 3.

In some embodiments, the compounds of the invention is a compound of Formula (III), Formula (IV), or Formula (V):

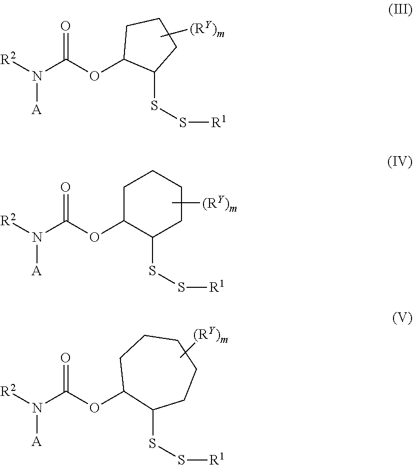

or a pharmaceutically acceptable salt thereof, wherein R¹, R², R^{Y}, A, and m are defined as in any of the embodiments above for Formula (II).

In some embodiments, the compound of formula (I) is selected from:

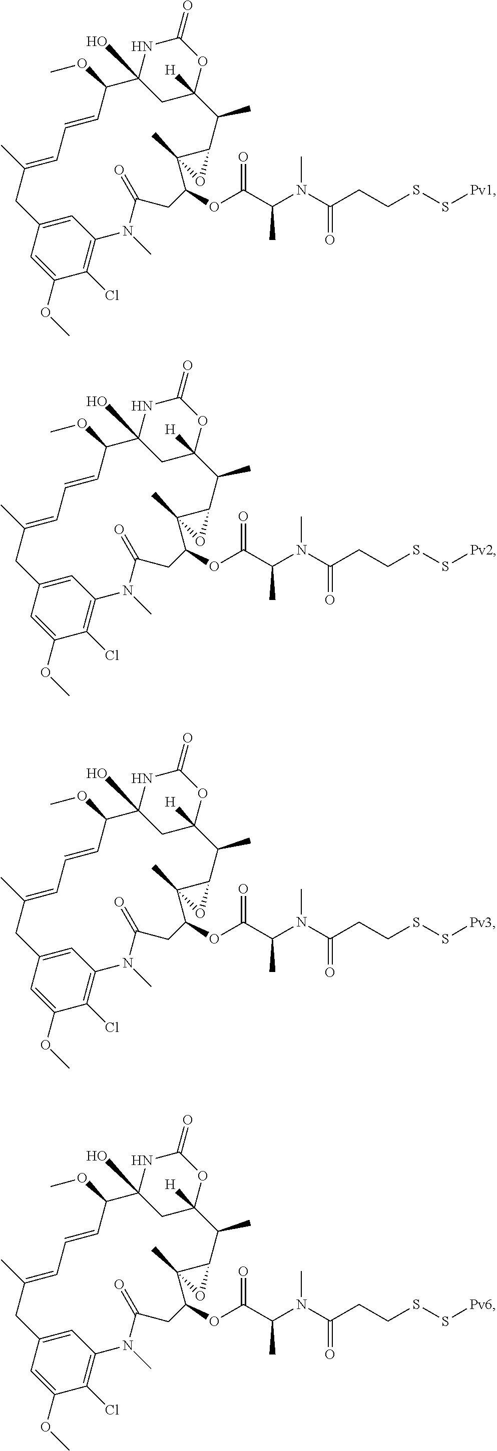

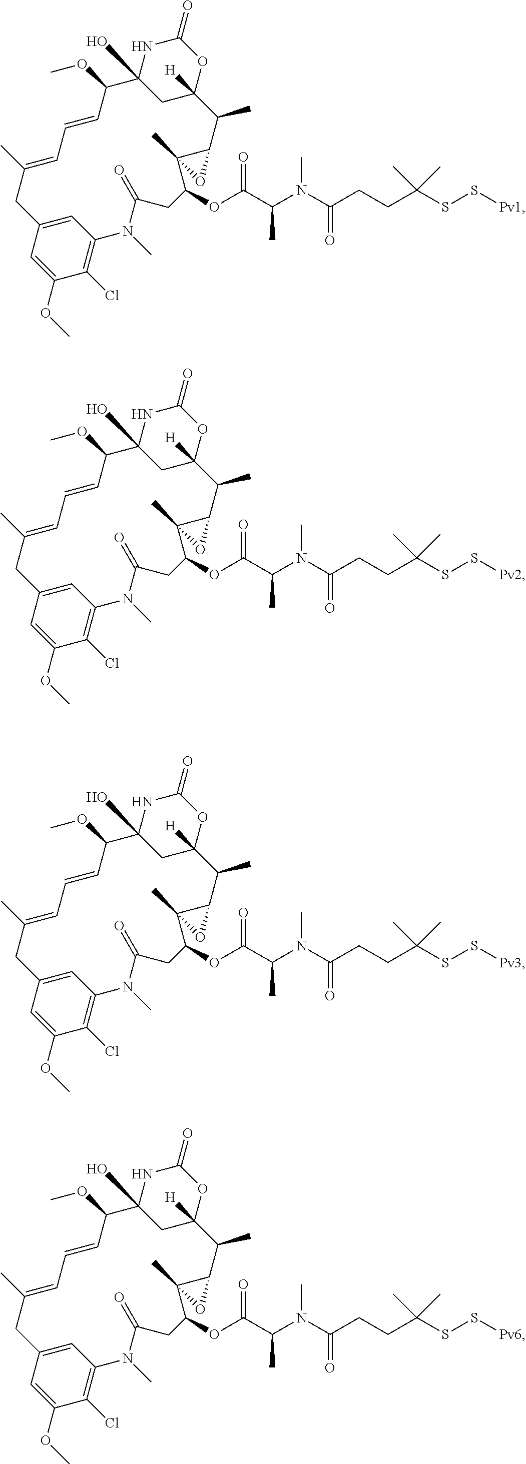

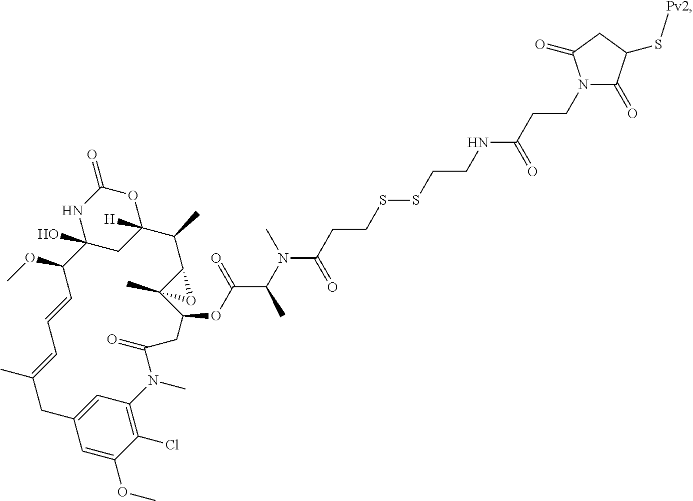

or a pharmaceutically acceptable salt of any of the aforementioned.

In some embodiments, the compound of Formula (I) is selected from:

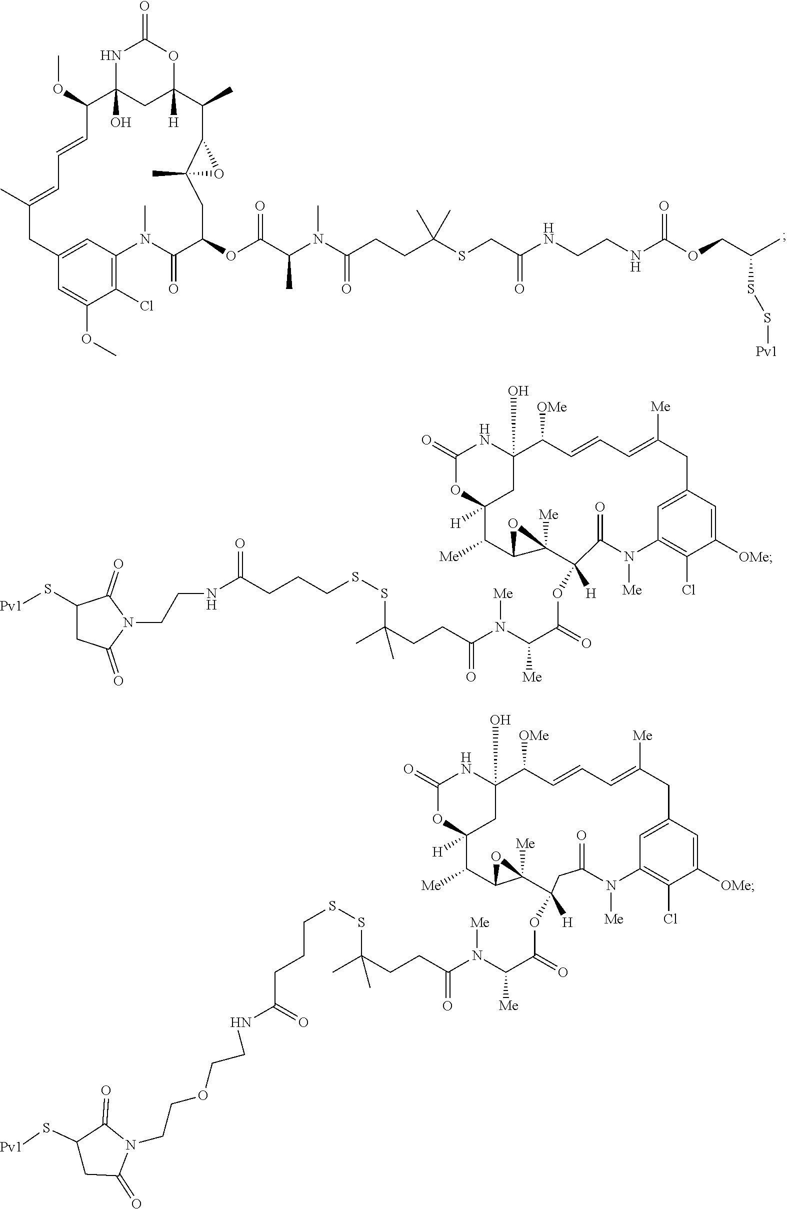

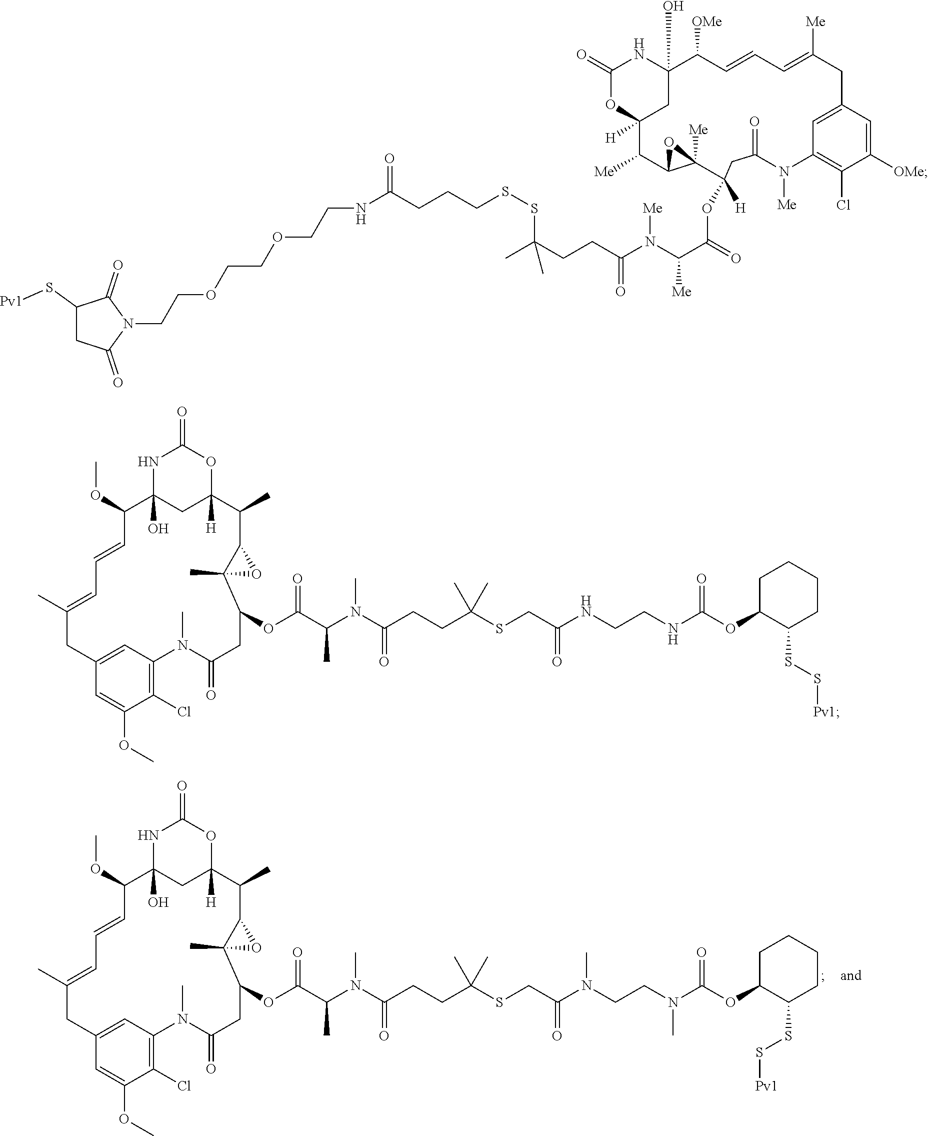

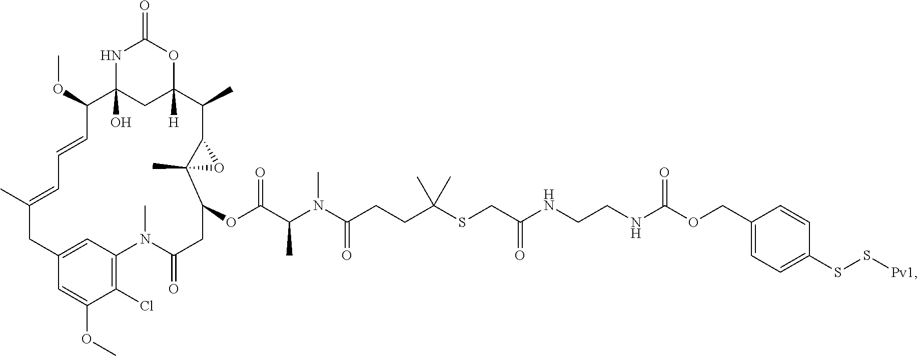

or a pharmaceutically acceptable salt of any of the aforementioned.

In some embodiments, provided herein is a compound having Formula (I-A):

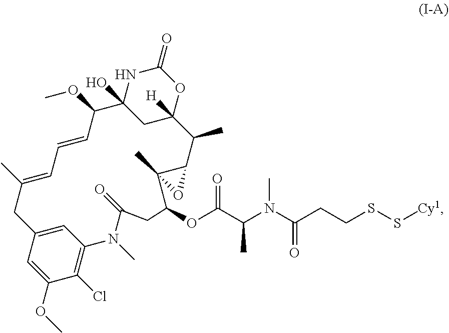

or a salt thereof, wherein Cy¹ is C₆₋₁₀ aryl or 5-10 membered heteroaryl. In some embodiments, Cy¹ is pyridyl.

In some embodiments, provided herein is a compound having Formula (I-B):

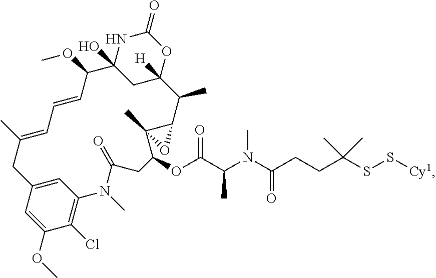

or a salt thereof, wherein Cy¹ is C₆₋₁₀ aryl or 5-10 membered heteroaryl. In some embodiments, Cy¹ is pyridyl.

The molecules of the invention can be tagged, for example, with a probe such as a fluorophore, radioisotope, and the like. In some embodiments, the probe is a fluorescent probe, such as LICOR. A fluorescent probe can include any moiety that can re-emit light upon light excitation (e.g., a fluorophore).

The Amino acids are represented by the IUPAC abbreviations, as follows: Alanine (Ala; A), Arginine (Arg; R), Asparagine (Asn; N), Aspartic acid (Asp; D), Cysteine (Cys; C), Glutamine (Gln; Q), Glutamic acid (Glu; E), Glycine (Gly; G), Histidine (His; H), Isoleucine (Ile; I), Leucine (Leu; L), Lysine (Lys; K), Methionine (Met; M), Phenylalanine (Phe; F), Proline (Pro; P), Serine (Ser; S), Threonine (Thr; T), Tryptophan (Trp; W), Tyrosine (Tyr; Y), Valine (Val; V).

The term “Pv1” means ADDQNPWRAYLDLLFPTDTLLLDLLWCG, which is the the peptide of SEQ ID No. 1.

The term “Pv2” means AEQNPIYWARYADWLFTTPLLLLDLALLVDADECG, which is the peptide of SEQ ID No. 2.

The term “Pv3” means ADDQNPWRAYLDLLFPTDTLLLDLLWDADECG, which is the peptide of SEQ ID No. 3.

The term “Pv4” means Ac-AAEQNPIYWARYADWLFTTPLLLLDLALLVDADEGTKCG, which is the peptide of SEQ ID NO. 4.

The term “Pv5” means AAEQNPIYWARYADWLFTTPLLLLDLALLVDADEGTC, which is the peptide of SEQ ID NO. 5. The term “Pv6” means AAEQNPIYWWARYADWLFTTPLLLLDLALLVDADEGTCG, which is the peptide of SEQ ID NO. 6. In the compounds of the invention, the peptides R¹ are attached to the disulfide linker by a cysteine moiety.

The term “acidic and/or hypoxic mantle” refers to the environment of the cell in the diseased tissue in question having a pH lower than 7.0 and preferably lower than 6.5. An acidic or hypoxic mantle more preferably has a pH of about 5.5 and most preferably has a pH of about 5.0. The compounds of formula (I) insert across a cell membrane having an acidic and/or hypoxic mantle in a pH dependent fashion to insert R²L into the cell, whereupon the disulfide bond of the linker is cleaved to deliver free R²L (or R²L*, wherein L* is a product of degradation). Since the compounds of formula (I) are pH-dependent, they preferentially insert across a cell membrane only in the presence of an acidic or hypoxic mantle surrounding the cell and not across the cell membrane of “normal” cells, which do not have an acidic or hypoxic mantle.

The terms “pH-sensitive” or “pH-dependent” as used herein to refer to the peptide R¹ or to the mode of insertion of the peptide R¹ or of the compounds of the invention across a cell membrane, means that the peptide has a higher affinity to a cell membrane lipid bilayer having an acidic or hypoxic mantle than a membrane lipid bilayer at neutral pH. Thus, the compounds of the invention preferentially insert through the cell membrane to insert R²L to the interior of the cell (and thus deliver R²H as described above) when the cell membrane lipid bilayer has an acidic or hypoxic mantle (a “diseased” cell) but does not insert through a cell membrane when the mantle (the environment of the cell membrane lipid bilayer) is not acidic or hypoxic (a “normal” cell). It is believed that this preferential insertion is achieved as a result of the peptide R¹ forming a helical configuration, which facilitates membrane insertion.

The term “small molecule microtubule targeting moiety” refers to a chemical group that binds to microtubules. The small molecule microtubule targeting moiety can be a group derived from a compound that inhibits the activity of microtubules. For example, the small molecule microtubule targeting moiety may suppress the dynamic stability of microtubules. In some embodiments, the small molecule microtubule targeting moiety has a molecular weight (Da) of about 100-1500, about 100-800, about 500-1,000, about 600-1,000, about 100-500, about 700-900, or about 250-500.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually.

Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene”, employed alone or in combination with other terms, refers to a divalent alkyl linking group. An alkylene group formally corresponds to an alkane with two C—H bond replaced by points of attachment of the alkylene group to the remainder of the compound.

The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “amino” refers to a group of formula —NH₂.

The term “carbonyl”, employed alone or in combination with other terms, refers to a —C(═O)— group, which also may be written as C(O).

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “haloalkoxy”, employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized □ (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring.

A five-membered heteroaryl ring is a heteroaryl group having five ring atoms wherein one or more (e.g., 1, 2 or 3) ring atoms are independently selected from N, O and S.

A six-membered heteroaryl ring is a heteroaryl group having six ring atoms wherein one or more (e.g., 1, 2 or 3) ring atoms are independently selected from N, O and S.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃-7). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 2-pyrrolidinyl, morpholinyl, azetidinyl, tetrahydrofuranyl, tetrahydropyranyl, and piperazinyl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as α-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et. al. J. Med. Chem. 2011, 54, 201-210; R. Xu et. al. J. Label Compd. Radiopharm. 2015, 58, 308-312).

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

The Schemes below provide general guidance in connection with preparing the compounds of the invention. One skilled in the art would understand that the preparations shown in the Schemes can be modified or optimized using general knowledge of organic chemistry to prepare various compounds of the invention.

Compounds of Formula (I) can be prepared, e.g., using a process as illustrated in the schemes below.

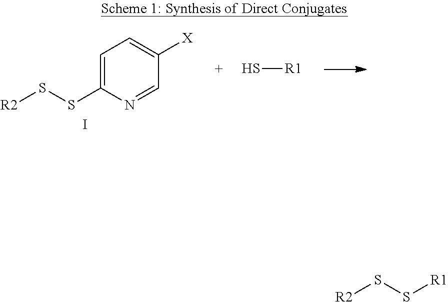

A small molecule microtubule targeting moiety which contains an inherent thiol (R²—SH), such as DM1 or DM4, can be activated by the formation of pyridyl disulfide (wherein X is, for example, H, halo, etc.), which can be displaced with a thiol containing R¹ peptide in a disulfide exchange reaction to give the desired conjugate where —S—S— is the linking moiety L.

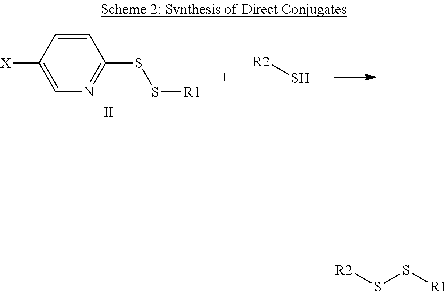

Alternatively, the R¹ peptide, which has an inherent thiol, can be activated by the formation of pyridyl disulfide (II) which can be displaced with a thiol-containing R² in a disulfide exchange reaction to give the desired direct conjugate where L is —S—S—.

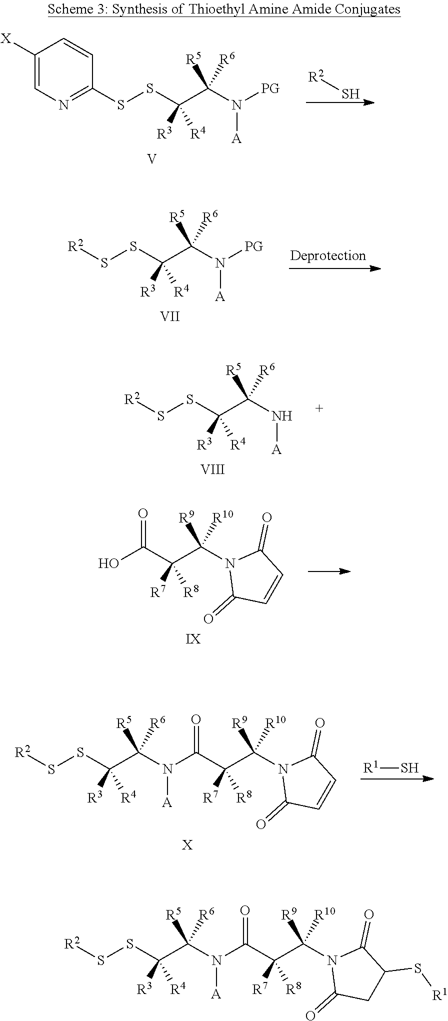

A protected ethyl amine, containing a thiol group that has been activated as pyridyl disulfide V can be reacted with thiol-containing R² in a disulfide exchange reaction to give VII. Disulfide VII can be deprotected to give VIII and further reacted with a propionic maleimide IX in an acid coupling reaction to provide amide X. Amide X can be reacted with a thiol containing peptide in a Michael addition to give the desired conjugate.

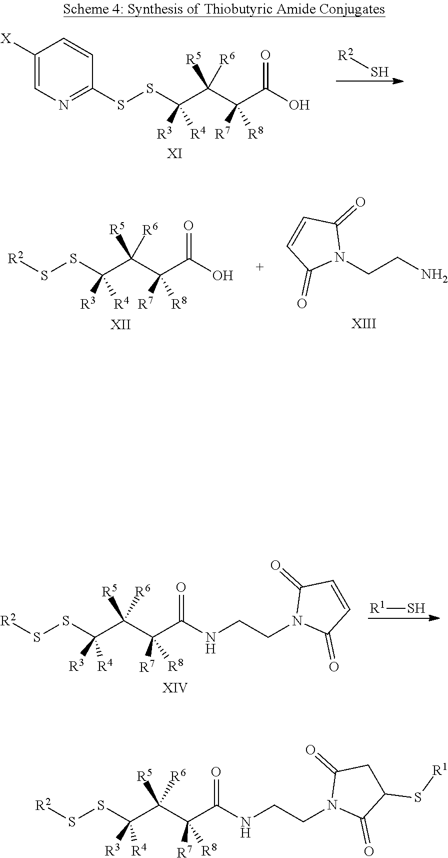

A thiol-containing butyric acid that has been activated as a pyridyl disulfide XI can be reacted with thiol containing R² in a disulfide exchange reaction to give XII. Disulfide acid XII can be reacted with ethylaminomaleimide XIII in an acid coupling reaction to provide amide XIV. Amide XIV can be reacted with a thiol-containing peptide in a Michael addition to give the desired conjugate.

The peptides R¹ may be prepared using the solid-phase synthetic method first described by Merrifield in J.A.C.S., Vol. 85, pgs. 2149-2154 (1963), although other art-known methods may also be employed. The Merrifield technique is well understood and is a common method for preparation of peptides. Useful techniques for solid-phase peptide synthesis are described in several books such as the text “Principles of Peptide Synthesis” by Bodanszky, Springer Verlag 1984. This method of synthesis involves the stepwise addition of protected amino acids to a growing peptide chain which was bound by covalent bonds to a solid resin particle. By this procedure, reagents and by-products are removed by filtration, thus eliminating the necessity of purifying intermediates. The general concept of this method depends on attachment of the first amino acid of the chain to a solid polymer by a covalent bond, followed by the addition of the succeeding protected amino acids, one at a time, in a stepwise manner until the desired sequence is assembled. Finally, the protected peptide is removed from the solid resin support and the protecting groups are cleaved off.

The amino acids may be attached to any suitable polymer. The polymer must be insoluble in the solvents used, must have a stable physical form permitting ready filtration, and must contain a functional group to which the first protected amino acid can be firmly linked by a covalent bond. Various polymers are suitable for this purpose, such as cellulose, polyvinyl alcohol, polymethylmethacrylate, and polystyrene.

### Methods of Use

Provided herein is the use of the compounds of formula (I) in the treatment of diseases, such as cancer or neurodegenerative disease. Another aspect of the present invention is the use of the compounds of formula (I) in the treatment of diseases involving acidic or hypoxic diseased tissue, such as cancer. Hypoxia and acidosis are physiological markers of many disease processes, including cancer. In cancer, hypoxia is one mechanism responsible for development of an acid environment within solid tumors. As a result, hydrogen ions must be removed from the cell (e.g., by a proton pump) to maintain a normal pH within the cell. As a consequence of this export of hydrogen ions, cancer cells have an increased pH gradient across the cell membrane lipid bilayer and a lower pH in the extracellular milieu when compared to normal cells. One approach to improving the efficacy and therapeutic index of cytotoxic agents is to leverage this physiological characteristic to afford selective delivery of compound to hypoxic cells over healthy tissue.

In these methods of treatment, a therapeutically-effective amount of a compound of formula (I) or a pharmaceutically-acceptable salt thereof may be administered as a single agent or in combination with other forms of therapy, such as ionizing radiation or cytotoxic agents in the case of cancer. In combination therapy, the compound of formula (I) may be administered before, at the same time as, or after the other therapeutic modality, as will be appreciated by those of skill in the art. Either method of treatment (single agent or combination with other forms of therapy) may be administered as a course of treatment involving multiple doses or treatments over a period of time.

Examples of cancers that are treatable using the compounds of the present disclosure include, but are not limited to, colorectal cancer, gastric cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In some embodiments, cancers treatable with compounds of the present disclosure include bladder cancer, bone cancer, glioma, breast cancer (e.g., triple-negative breast cancer), cervical cancer, colon cancer, colorectal cancer, endometrial cancer, epithelial cancer, esophageal cancer, Ewing's sarcoma, pancreatic cancer, gallbladder cancer, gastric cancer, gastrointestinal tumors, head and neck cancer (upper aerodigestive cancer), intestinal cancers, Kaposi's sarcoma, kidney cancer, laryngeal cancer, liver cancer (e.g., hepatocellular carcinoma), lung cancer (e.g., non-small cell lung cancer, adenocarcinoma), melanoma, prostate cancer, rectal cancer, renal clear cell carcinoma, skin cancer, stomach cancer, testicular cancer, thyroid cancer, and uterine cancer.

In some embodiments, cancers treatable with compounds of the present disclosure include melanoma (e.g., metastatic malignant melanoma), renal cancer (e.g. clear cell carcinoma), prostate cancer (e.g. hormone refractory prostate adenocarcinoma), breast cancer, triple-negative breast cancer, colon cancer and lung cancer (e.g. non-small cell lung cancer and small cell lung cancer). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In certain embodiments, a compound of formula (I) or a pharmaceutically-acceptable salt thereof may be used in combination with a chemotherapeutic agent, a targeted cancer therapy, an immunotherapy or radiation therapy. The agents can be combined with the present compounds in a single dosage form, or the agents can be administered simultaneously or sequentially as separate dosage forms. In some embodiments, the chemotherapeutic agent, targeted cancer therapy, immunotherapy or radiation therapy is less toxic to the patient, such as by showing reduced bone marrow toxicity, when administered together with a compound of formula (I), or a pharmaceutically acceptable salt thereof, as compared with when administered in combination with the corresponding microtubule targeting agent (e.g., R²—H).

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with the compounds of the present invention include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds according to the invention may also be combined with immunotherapy drugs, including cytokines such as interferon alpha, interleukin 2, and tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (TAXOL™), mithramycin, deoxycoformycin, mitomycin-C, L-asparaginase, interferons (especially IFN-α), etoposide, and teniposide.

Other cytotoxic agents that can be administered in combination with the compounds of the invention include, for example, navelbene, CPT-11, anastrazole, letrazole, capecitabine, reloxafine, cyclophosphamide, ifosamide, and droloxafine.

Also suitable are cytotoxic agents such as, for example, epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB and PD-1, or antibodies to cytokines (IL-10, TGF-α, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines that can be administered in combination with the compounds of the invention include, for example, dendritic cells, synthetic peptides, DNA vaccines and recombinant viruses.

Other suitable agents for use in combination with the compounds of the present invention include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles (Abraxane®).

Compounds of this invention may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present invention. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Compounds of the present invention may be combined with or administered in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with the compounds of the invention. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with compounds of the invention. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds of the present invention include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds of the invention. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds of the present invention. A further example of a PARP inhibitor that can be combined with a compound of the invention is talazoparib.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

The phrase “therapeutically effective amount” of a compound (therapeutic agent, active ingredient, drug, etc.) refers to an amount of the compound to be administered to a subject in need of therapy or treatment which alleviates a symptom, ameliorates a condition, or slows the onset of disease conditions, according to clinically acceptable standards for the disorder or condition to be treated. For instance, a therapeutically effective amount can be an amount which has been demonstrated to have a desired therapeutic effect in an in vitro assay, an in vivo animal assay, or a clinical trial. The therapeutically effective amount can vary based on the particular dosage form, method of administration, treatment protocol, specific disease or condition to be treated, the benefit/risk ratio, etc., among numerous other factors.

Said therapeutically effective amount can be obtained from a clinical trial, an animal model, or an in vitro cell culture assay. It is known in the art that the effective amount suitable for human use can be calculated from the effective amount determined from an animal model or an in vitro cell culture assay. For instance, as reported by Reagan-Shaw et al., FASEB J. 2008: 22(3) 659-61, “μg/ml” (effective amount based on in vitro cell culture assays)=“mg/kg body weight/day” (effective amount for a mouse). Furthermore, the effective amount for a human can be calculated from the effective amount for a mouse based on the fact that the metabolism rate of mice is 6 times faster than that of humans.

As an example of treatment using a compound of formula (I) in combination with a cytotoxic agent, a therapeutically-effective amount of a compound of formula (I) may be administered to a patient suffering from cancer as part of a treatment regimen also involving a therapeutically-effective amount of ionizing radiation or a cytotoxic agent. In the context of this treatment regimen, the term “therapeutically-effective” amount should be understood to mean effective in the combination therapy. It will be understood by those of skill in the cancer-treatment field how to adjust the dosages to achieve the optimum therapeutic outcome.

Similarly, the appropriate dosages of the compounds of the invention for treatment of non-cancerous diseases or conditions (such as cardiovascular diseases) may readily be determined by those of skill in the medical arts.

The term “treating” as used herein includes the administration of a compound or composition which reduces the frequency of, delays the onset of, or reduces the progression of symptoms of a disease involving acidic or hypoxic diseased tissue, such as cancer, stroke, myocardial infarction, or long-term neurodegenerative disease, in a subject relative to a subject not receiving the compound or composition. This can include reversing, reducing, or arresting the symptoms, clinical signs, or underlying pathology of a condition in a manner to improve or stabilize a subject's condition (e.g., regression of tumor growth, for cancer or decreasing or ameliorating myocardial ischemia reperfusion injury in myocardial infarction, stroke, or the like cardiovascular disease). The terms “inhibiting” or “reducing” are used for cancer in reference to methods to inhibit or to reduce tumor growth (e.g., decrease the size of a tumor) in a population as compared to an untreated control population.

All publications (including patents) mentioned herein are incorporated herein by reference for the purpose of describing and disclosing, for example, the constructs and methodologies that are described in the publications, which might be used in connection with the disclosure herein described. The publications discussed throughout the text are provided solely for their disclosure prior to the filing date of the present application.

Disclosed herein are several types of ranges. When a range of any type is disclosed or claimed, the intent is to disclose or claim individually each possible number that such a range could reasonably encompass, including end points of the range as well as any sub-ranges and combinations of sub-ranges encompassed therein. When a range of therapeutically effective amounts of an active ingredient is disclosed or claimed, for instance, the intent is to disclose or claim individually every possible number that such a range could encompass, consistent with the disclosure herein. For example, by a disclosure that the therapeutically effective amount of a compound can be in a range from about 1 mg/kg to about 50 mg/kg (of body weight of the subject).

### Formulation, Dosage Forms and Administration

To prepare the pharmaceutical compositions of the present invention, a compound of Formula (I) or a pharmaceutically-acceptable salt thereof is combined as the active ingredient in intimate admixture with a pharmaceutical carrier according to conventional pharmaceutical compounding techniques, which carrier may take a wide variety of forms depending on the form of preparation desired for administration, e.g., oral or parenteral. In preparing the compositions in oral dosage form, any of the usual pharmaceutical media may be employed, such as for example, water, glycols, oils, alcohols, flavoring agents, preservatives, coloring agents, and the like in the case of oral liquid preparations such as for example, suspensions, elixirs, and solutions; or carriers such as starches, sugars, diluents, granulating agents, lubricants, binders, disintegrating agents, and the like in a case of oral solid preparations, such as for example, powders, capsules, and tablets. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, although other ingredients, for example, to aid solubility or for preservative purposes, may be included. Injectable suspensions may also be prepared, in which case appropriate liquid carriers, suspending agents, and the like may be employed. One of skill in the pharmaceutical and medical arts will be able to readily determine a suitable dosage of the pharmaceutical compositions of the invention for the particular disease or condition to be treated.

### EXAMPLES

As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997. The following definitions describe terms and abbreviations used herein:
Brine: a saturated NaCl solution in water
DCM: dichloromethane
TFA: trifluoroacetic acid
DIPEA: diisopropylethylamine
DMA: dimethylacetamide
DME: dimethoxyethane
DMF: dimethylformamide
DMSO: methylsulfoxide
DTT: dithiothreitol
MSD: mass spec detector
Et₂O: ethyl ether
EtOAc: ethyl acetate
EtOH: ethyl alcohol
HATU: O-(7-azabenzotriazol-1-yl)-N,N,N′,N′-tetramethyluronium hexafluorophosphate
HOBt: 1-hydroxybenzotriazole
RP: reverse phase
HPLC: high performance liquid chromatography
IPA: isopropanol
LAH: lithium aluminum hydride
N-BuLi: n-butyl lithium
LC-MS: liquid chromatography-mass spectrometry
LDA: lithium diisoproylethylamide
Me: methyl
MeOH: methanol
MTBE: methyl t-butyl ether
NMP: N-methylpyrrolidine
Ph: phenyl
PNPC: para-nitrophenylchloroformate
RT or rt: room temperature
SFC: supercritical fluid chromatography
TBAI: tetrabutylammonium iodide
TBME: tert-butylmethyl ether
tBu: tertiary butyl
THF: tetrahydrofuran
TEA: triethylamine
TMEDA: tetramethylethylenediamine
GSH: Glutathione
GS: Glutathione bonded at sulfur
LiOH: lithium hydroxide
DPPA: diphenyl phosphoryl azide
Sn(Bu)₂(Laurate)₂: dibutyltin dilaurate
PBS: phosphate buffered saline
ACN: acetonitrile
AcOH: acetic acid
EEDQ: N-ethoxycarbonyl-2-ethoxy-1,2-dihydroquinoline
DMAP: 4-dimethylaminopyridine
EDC: 1-ethyl-3-(3-dimethylaminopropyl)carbodiimide
   The HPLC methods employed are set forth below.

HPLC Methods
A: Sunfire C18 150×4.6 mm; H₂O/Acetonitrile w/TFA modifier (0.05%); Flow rate: 1 ml/min; Wavelength=217 nM.
B: Ace Equivalence 250×4.6 mm; H₂O/Acetonitrile w/TFA modifier (0.05%); Flow rate: 1 ml/min; Wavelength=217 nM.
C: Sunfire C18 150×30 mm; H₂O/Acetonitrile w/TFA modifier (0.05%); Flow rate: 30 ml/min; Wavelength=217 nM.
D: Sunfire C18 150×4.6 mm; H₂O/Acetonitrile w/AcOH modifier (0.5%); Flow rate: 1 ml/min; Wavelength=217 nM
E: Sunfire C18 150×30 mm; H₂O/Acetonitrile w/AcOH modifier (0.5%); Flow rate: 30 ml/min; Wavelength=217 nM.
F: Agilent 1100/1200/1260 or 1290 systems (coupled or uncoupled with MS).

HPLC Parameters

| Mobile Phase A | 0.1% AcOH in water | |
|---|---|---|
| Mobile Phase B | 0.1% AcOH in ACN | |
| Column | Merck Chromolith RP-18e | |
| Column Temperature | rt | |
| Autosampler | rt | |
| Temperature | | |
| Injection Volume | 5 | μL |
| Flow Rate | 1 | mL/minute |

| Wavelength | Agilent diode array detector at λ = 254, | | |
|---|---|---|---|
| | 220 or 280 nm | | |
| Gradient Program | Time (min) | % A | % B |
| | Initial | 95.00 | 5.00 |
| | 4.00 | 5.00 | 95.00 |
| | 4.99 | 5.00 | 95.00 |
| | 5.00 | 95.00 | 5.00 |
| | 6.00 | 95.00 | 5.00 |

| | |
|---|---|
| Run Time | 6.00 |

G: Agilent 1100/1200/1260 or 1290 systems (coupled or uncoupled with MS).

HPLC Parameters

| Mobile Phase A | 0.1% TFA in water | |
|---|---|---|
| Mobile Phase B | 0.1% TFA in ACN | |
| Column | Agilent Eclipse XDB C8 column | |
| | (3.5 μm, 4.6 × 150 mm) | |
| Column Temperature | 40° C. | |
| Autosampler | rt | |
| Temperature | | |
| Injection Volume | 5 | μL |
| Flow Rate | 1.5 | mL/minute |

| Wavelength | Agilent diode array detector at λ = 254, | | |
|---|---|---|---|
| | 220 or 280 nm | | |
| Gradient Program | Time (min) | % A | % B |
| | Initial | 80.00 | 20.00 |
| | 0.20 | 80.00 | 20.00 |
| | 7.50 | 20.00 | 80.00 |
| | 8.00 | 0.00 | 100.00 |
| | 9.00 | 0.00 | 100.00 |
| | 10.00 | 80.00 | 20.00 |

| | |
|---|---|
| Run Time | 10.00 |

Mass Spectrometry Methods

Maldi-TOF (Matrix-assisted laser desorption/ionization-Time of Flight) mass spectrometry was measured on an Applied Biosystems Voyager System 6268. The sample was prepared as a matrix of α-cyano hydroxy cinnamic acid on an AB Science plate (Part #V700666).

ESI (Electrospray Ionization) mass spectrometry was measured on either an Agilent 1100 series LC-MS with a 1946 MSD or a Waters Xevo Qtof high-resolution MS, both providing a mass/charge species (m/z=3).

The source of the starting materials employed in the Examples are set forth below in the following tables.

TABLE 2
Starting materials for R²

| | | Synthesis, Reference or |
|---|---|---|
| R² Code | R²H Structure | Purchased |
| R²SH-1 | 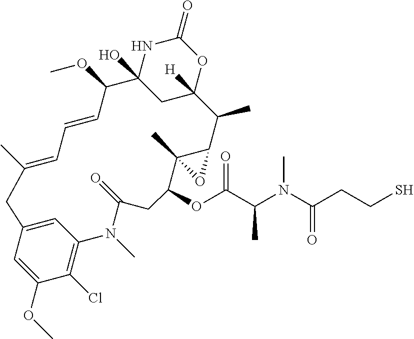 | MedKoo 123212 |
| R²SH-2 | 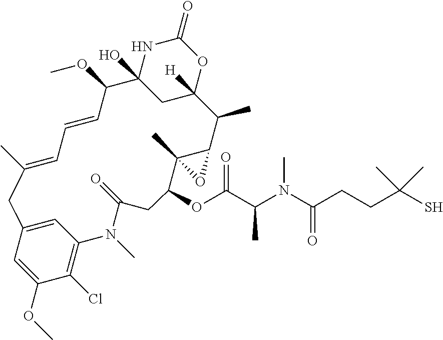 | MedKoo 206839 |
| R²SH-3 | 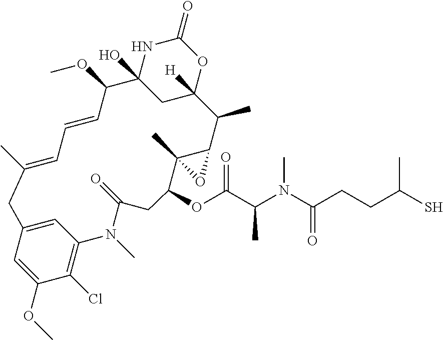 | US 20040235840 A1 |

### Synthesis of Intermediate I (R²S—S-Pyr)

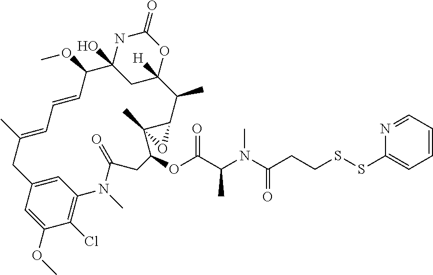

To [(1S,2R,3 S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[methyl(3-sulfanylpropanoyl)amino]propanoate (46.7 mg, 0.06 mmol) in 1 mL of CH₃CN was added 2-(2-pyridyldisulfanyl)pyridine (20.0 mg, 0.09 mmol). The mixture was concentrated and purified (SiO₂, 0-10% MeOH/CH₂Cl₂) to give [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[methyl-[3-(2-pyridyldisulfanyl)propanoyl]amino]propanoate (53.6 mg, yield: 100%). MS m/z 847.1 [M+H]⁺.

### Synthesis of Pv3-S-Pyr (Intermediate II-3)

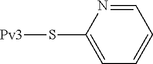

Pv3 (250 mg, 0.06 mmol; as a free flowing solid) and 2-(2-pyridyldisulfanyl)pyridine (0.110 g, 0.5 mol) were dissolved in MeOH (10 mL) and the reaction stirred overnight at room temperature. LC-MS indicates the desired product was formed. The reaction mixture was concentrated and the residue taken up in DMSO and purified by reverse phase column chromatography (40-65% CH₃CN/H₂O (0.5% AcOH), 13 min) to give 212 mg of the desired product (187 mg, yield: 74.9%). MS m/z=3 1273.4.

Intermediates II-1, II-2 and II-6 were prepared analogously to II-3, using Pv1, Pv2, and Pv6, as shown below:

| | | MS |
|---|---|---|
| | | A: Maldi-TOF |
| Intermediate | Structure | B: m/z = 3 |
| II-1 | Pv1-SPyr | B: 1130.1 |
| II-2 | Pv2-SPyr | B: 1373.9 |
| II-3 | Pv3-SPyr | B: 1273.4 |
| II-6 | Pv6-SPyr | B: 1450.3 |

TABLE 3
Starting materials for L groups

| | | Purchased, Reference |
|---|---|---|
| Intermediate | Structure | or Synthesized |
| III-1 | 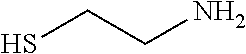 | Enamine EN3000-33931 |
| III-2 | 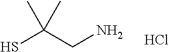 | Astatech 39541 |
| III-3 | 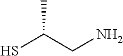 | Enamine EN3000-6731388 |
| III-4 | 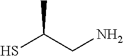 | Enamine EN3000-6731596 |
| III-5 | 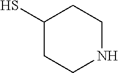 | Astatech 39018 |

### Synthesis of Intermediate VI-2

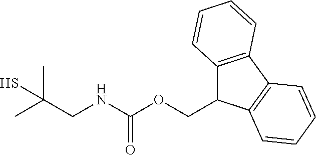 [SMILES: CC(C)(S)CNC(=O)OCC1c2ccccc2-c2ccccc21]

1-Amino-2-methyl-propane-2-thiol hydrochloride (100 mg, 0.706 mmol) was dissolved in CH₂Cl₂ (7 mL) and to it was added 9H-fluoren-9-ylmethyl carbonochloridate (274 mg, 1.06 mmol) and N,N-diisopropylethylamine (182 mg, 1.41 mmol). The reaction mixture was stirred at room temperature overnight. The reaction mixture was washed with water and concentrated. The residue was purified by column chromatography (0-50% EtOAc/hexanes) to give 9H-fluoren-9-ylmethyl N-(2-methyl-2-sulfanyl-propyl)carbamate (213 mg, yield: 92.2%). MS m/z 350.1 [M+Na]⁺.

### Synthesis of Intermediate V-1

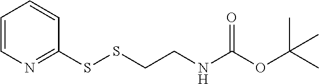 [SMILES: CC(C)(C)OC(=O)NCCSSc1ccccn1]

2-(2-Pyridyldisulfanyl)pyridine (746 mg, 3.38 mmol) was dissolved in MeOH (15 mL) and to it was added tert-butyl N-(2-sulfanylethyl)carbamate (200 mg, 1.13 mmol). The reaction was stirred for 3 h at room temperature. The mixture was concentrated and the residue purified by column chromatography (0-50% EtOAc/hexanes) to give tert-butyl N-[2-(2-pyridyldisulfanyl)ethyl]carbamate (200 mg, yield: 61.9%). MS m/z 287.1 [M+H]⁺.

### Synthesis of Intermediate V-2

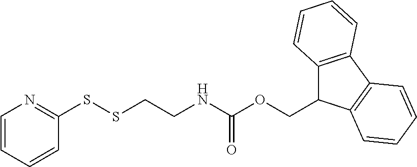 [SMILES: O=C(NCCSSc1ccccn1)OCC1c2ccccc2-c2ccccc21]

2-(2-Pyridyldisulfanyl)ethanamine hydrochloride (200 mg, 0.898 mmol) was dissolved in CH₂Cl₂ and to it was added 9H-fluoren-9-ylmethyl carbonochloridate (348 mg, 1.35 mmol) and N,N-diisopropylethylamine (232 mg, 1.80 mmol). The reaction mixture was stirred at RT for 2 h, washed with water and concentrated. The residue was purified by column chromatography (0-50% EtOAc/hexanes) to give 9H-fluoren-9-ylmethyl N-[2-(2-pyridyldisulfanyl)ethyl]carbamate (288 mg, yield: 78.5%). MS m/z 409.1 [M+H]⁺.

### Synthesis of Intermediate VII-1

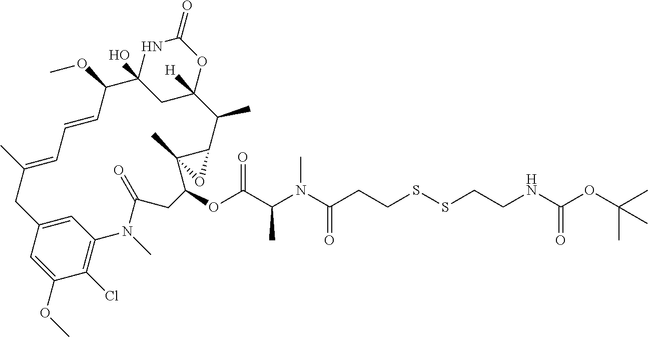

To a vial containing [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[methyl(3-sulfanylpropanoyl)amino]propanoate (25.0 mg, 0.03 mmol) in 1 mL of CH₃CN was added tert-butyl N-[2-(2-pyridyldisulfanyl)ethyl]carbamate (Intermediate V-1, 14.5 mg, 0.051 mmol) and 4-methylmorpholine (0.138 mL, 1.25 mmol). The mixture was stirred for 16 h. LC-MS analysis indicates the desired material. The mixture was concentrated, dissolved in 50 mL of EtOAc and washed with 1×25 mL of sat. NH₄Cl and 1×25 mL of sat. brine. The organic phase was dried with MgSO₄, filtered and concentrated. The crude residue was purified (SiO₂, 0-100% EtOAc/hexanes) to give [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-[2-(tert-butoxycarbonylamino)ethyldisulfanyl]propanoyl-methyl-amino]propanoate (30.9 mg, yield: 100%). MS m/z 913.2 [M+H]⁺.

### Synthesis of Intermediate VII-2

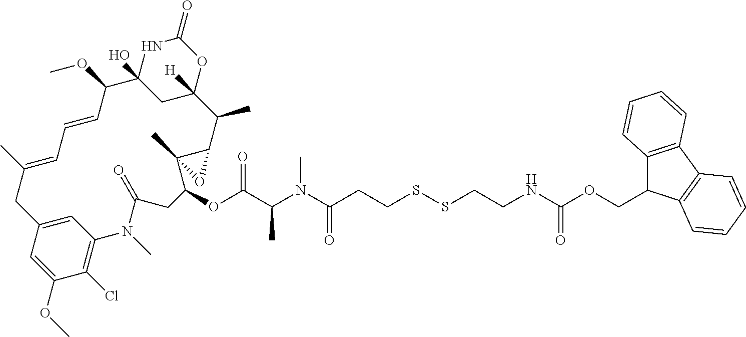

Intermediate VII-2 was prepared analogously to VII-1, using Intermediate V-2 in place of Intermediate V-1.

### Synthesis of Intermediate VII-3

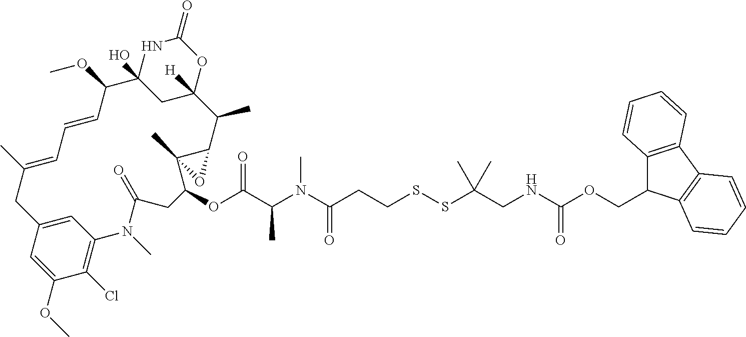

To a vial containing [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-[2-(9H-fluoren-9-ylmethoxycarbonylamino)ethyldisulfanyl]propanoyl-methyl-amino]propanoate (25.0 mg, 0.03 mmol) in 1 mL of CH₃CN was added 9H-fluoren-9-ylmethyl N-(2-methyl-2-sulfanyl-propyl)carbamate (14.5 mg, 0.044 mmol) and 4-methylmorpholine (0.120 mL, 1.09 mmol). The mixture was stirred for 16 h. LC-MS analysis indicated the desired material was formed. The mixture was concentrated, dissolved in 50 mL of EtOAc and washed with 1×25 mL of sat. NH₄Cl and 1×25 mL of sat. brine. The organic phase was dried with MgSO₄, filtered and concentrated. The crude residue was purified (SiO₂, 0-100% EtOAc/hexanes) to [(1S,2R,3 S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-[[2-(9H-fluoren-9-ylmethoxycarbonylamino)-1,1-dimethyl-ethyl]disulfanyl]propanoyl-methyl-amino]propanoate (0.0313 g, yield: 100%). MS m/z 1085.0 [M+Na]⁺.

### Synthesis of Intermediate VIII-1 (BOC Deprotection)

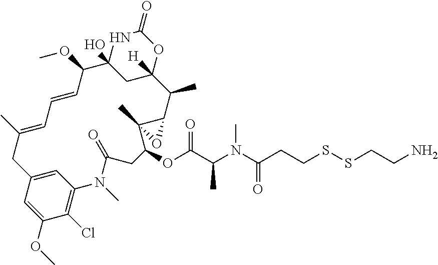

[(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-[2-(tert-butoxycarbonylamino)ethyldisulfanyl]propanoyl-methyl-amino]propanoate (31.9 mg, 0.05 mmol) was dissolved in 0.3/0.1/0.1 mL of CH₃CN/H₂O/TFA. The mixture was stirred for 36 h. LC-MS indicated complete deprotection. The mixture was purified by prep HPLC (20-95% CH₃CN/H₂O w/0.05% TFA) to give [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-(2-aminoethyldisulfanyl)propanoyl-methyl-amino]propanoate;2,2,2-trifluoroacetic acid (22.9 mg, yield: 70.7%). MS m/z 813.2 [M+H]⁺.

### Alternative Synthesis of Intermediate VIII-1 (FMOC Deprotection)

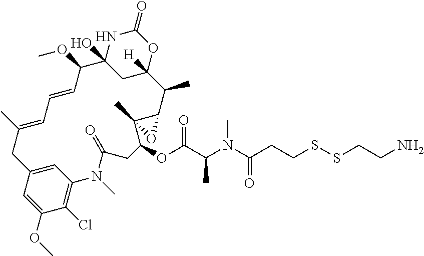

To a vial containing [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-[2-(9H-fluoren-9-ylmethoxycarbonylamino)ethyldisulfanyl]propanoyl-methyl-amino]propanoate (Intermediate VII-2; 29.6 mg, 0.03 mmol) was added 0.5 mL of DMF and 4-methylmorpholine (0.120 mL, 1.09 mmol). The mixture was heated for 16 h at 40° C. LC-MS confirmed complete deprotection. The mixture was purified by (20-95% CH₃CN/H₂O w/0.05% TFA) to give [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-(2-aminoethyldisulfanyl)propanoyl-methyl-amino]propanoate trifluoroacetate (22.9 mg, yield: 86.4%). MS m/z 813.2 [M+H]⁺.

### Synthesis of Intermediate VIII-2

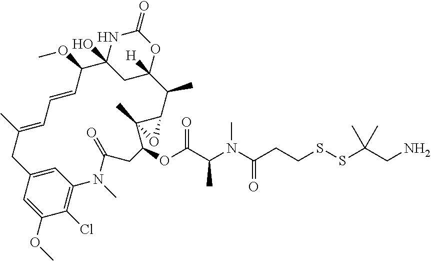

Intermediate VIII-2 was prepared analogously to Intermediate VIII-1. MS m/z 841.2 [M+H]⁺.

### Synthesis of Intermediate X-1

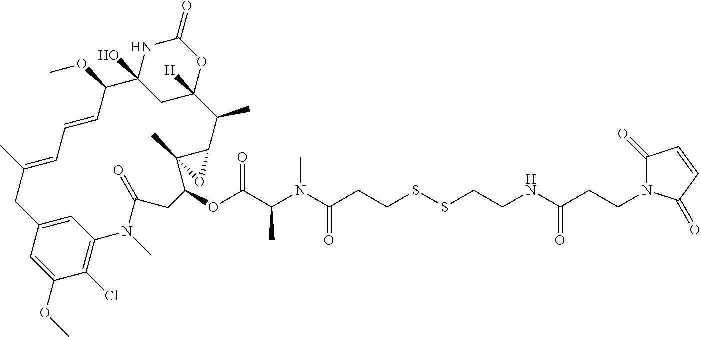

To [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-(2-aminoethyldisulfanyl)propanoyl-methyl-amino]propanoate trifluoroacetate (Intermediate VIII-1; 45.8 mg, 0.05 mmol) in 1 mL of DMF was added 3-(2,5-dioxopyrrol-1-yl)propanoic acid (12.5 mg, 0.074 mmol), TBTU (23.8 g, 0.074 mmol) and DIPEA (0.0169 mL, 0.1 mmol). LC-MS indicated complete conversion to the product. The mixture was diluted with 50 mL of EtOAc. This was washed with 1×25 mL sat NH₄Cl, 4×25 mL H₂O and 1×25 mL of H₂O. The organic phase was dried with MgSO₄, filtered and concentrated. The crude product was purified (SiO₂, 0-10% MeOH/CL₂Cl₂) to give [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-[2-[3-(2,5-dioxopyrrol-1-yl)propanoylamino]ethyldisulfanyl]propanoyl-methyl-amino]propanoate (17.3 mg, yield: 36.5%) MS m/z 986.1 [M+Na]⁺.

### Example 2: Synthesis of Compound 2

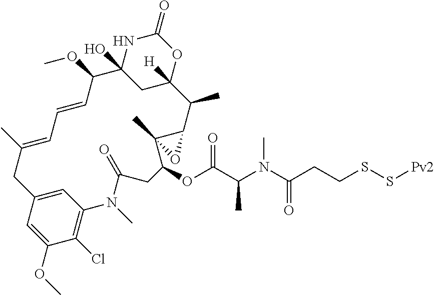

To a vial containing Pv2 (25.0 mg, 0.006 mmol; as a free flowing solid) and [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[methyl-[3-(2-pyridyldisulfanyl)propanoyl]amino]propanoate (7.70 mg, 0.009 mmol) was added 1 mL of degassed DMF and 0.5 mL degassed H₂O. To this was added CH₃CO₂H (0.0103 mL, 0.180 mmol). The mixture was stirred for 72 h. LC-MS indicated formation of desired product. The mixture was purified by prep HPLC (Sunfire C18 150×30 mm; 20-77% H₂O/Acetonitrile w/0.5% AcOH modifier; 15 min run; Flow rate: 30 ml/min; Wavelength=217 nM) to give the desired conjugate (17.0 mg, yield: 59.1%).

### Example 6: Synthesis of Compound 6

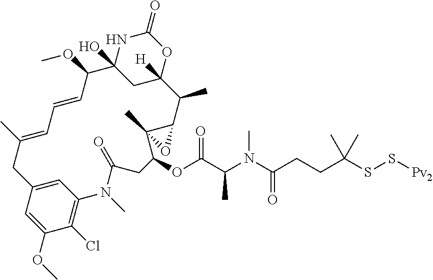

To a vial containing Pv2-SPyr (Intermediate II-2; 27.0 mg, 6.55e-6 mol) and [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10,12,14(26),16,18-pentaen-6-yl] (2S)-2-[methyl-(4-methyl-4-sulfanyl-pentanoyl)amino]propanoate (7.67 mg, 0.01 mmol). To this was added 1 mL of degassed DMF and 0.5 mL degassed H₂O. To this was added CH₃CO₂H (0.015 mL, 0.262 mmol). The mixture was stirred for 72 h. LC-MS indicated formation of desired product. The mixture was purified by prep HPLC (Sunfire C18 150×30 mm; 20-80% H₂O/Acetonitrile w/0.5% AcOH modifier; 16 min run; Flow rate: 30 ml/min; Wavelength=217 nM) to give the desired conjugate (11.4 g, yield: 36.6%).

### Example 9. Synthesis of Compound 9

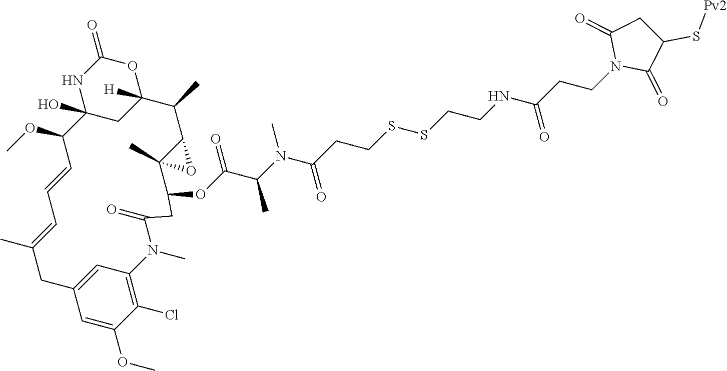

To a vial containing Pv2 (25.0 mg, 0.006 mol; as a free flowing solid) and [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10(26),11,13,16,18-pentaen-6-yl] (2S)-2-[3-[2-[3-(2,5-dioxopyrrol-1-yl)propanoylamino]ethyldisulfanyl]propanoyl-methyl-amino]propanoate (Intermediate X-1; 0.00877 g, 0.01 mmol) was added 1 mL of CH₃CN. The mixture was heterogeneous. To this was added 0.5 mL of CH₃CN, 0.5 mL of H₂O and 0.5 mL of MeOH. Homogeneity was not achieved. The mixture was stirred rigorously for 72 h. LC-MS indicated formation of desired product. The mixture was purified by prep HPLC (Sunfire C18 150×30 mm; 45-61% H₂O/Acetonitrile w/0.05% TFA modifier; 13 min run; Flow rate: 30 ml/min; Wavelength=217 nM) to give desired conjugate (21.1 mg, yield: 70.0%).

Compounds 1, 3, and 4 were synthesized analogously to the compound of Compound 2, using Pv1, Pv3, and Pv4, respectively. Compounds 5, 7, and 8 were synthesized analogously to Compound 6, using Intermediates II-1, II-3, and II-6, respectively.

TABLE 4
Example Compounds

| | | MS | |
|---|---|---|---|
| | | A: Maldi- | Conditions |
| | | TOF (M+) | % ACN/H₂O |
| | | B: ESI | Run Time |
| Compound | Structure | (m/z = 3) | RT |
| 1 | 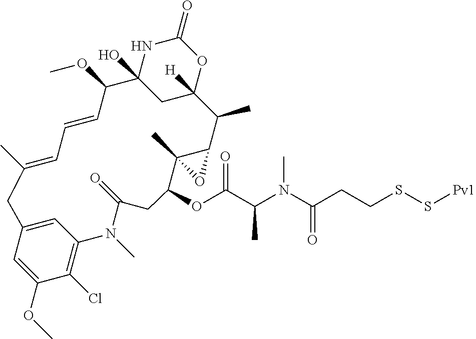 | B: 1339.3 | D 20-95% 11 min 7.28 min |
| 2 | 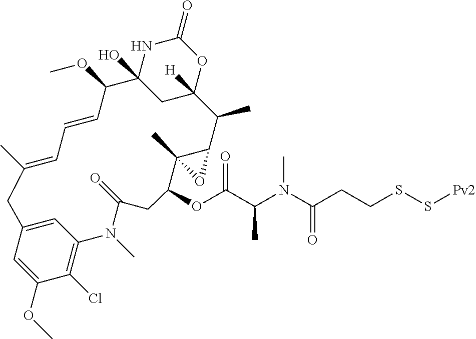 | B: 1582.7 | A 20-95% 11 min 7.0 min |
| 3 | 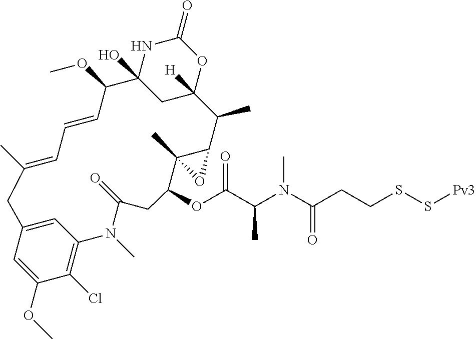 | B: 1483.8 | D 20-95% 11 min 7.82 min |
| 4 | 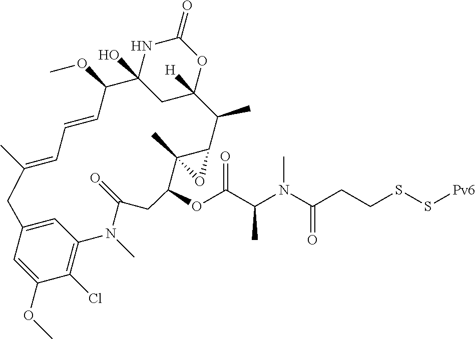 | B: 1659.4 | A 20-95% 11 min 6.92 min |
| 5 | 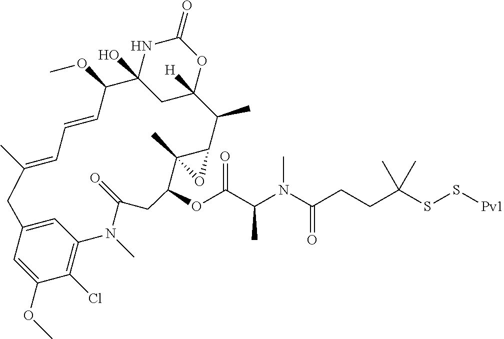 | B: 1352.8 | A 20-95% 11 min 6.81 min |
| 6 | 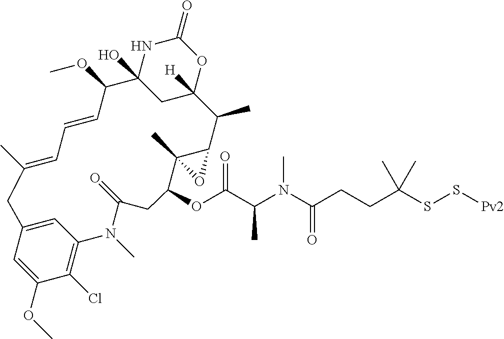 | B: 1597.0 | A 20-95% 11 min 7.44 min |
| 7 | 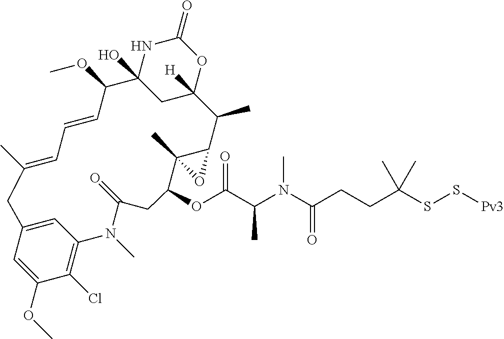 | B: 1487.1 | A 20-95% 11 min 6.60 min |
| 8 | 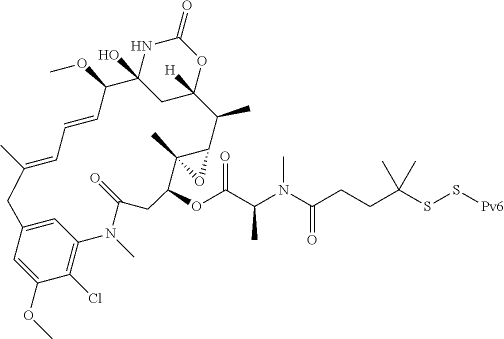 | B: 1673.4 | A 20-95% 11 min 7.14 min |
| 9 | 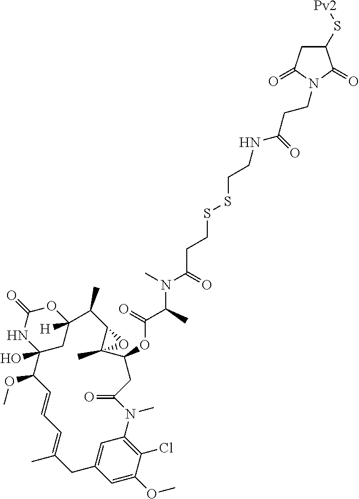 | B: 1659.4 | A 20-95% 11 min 7.16 min |

### Example 5: Detailed Synthesis of Compound 5

To Pv1 (50.0 mg, 1.48e-5 mol) and [(1S,2R,3S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10,12,14(26),16,18-pentaen-6-yl] (2S)-2-[methyl-(4-methyl-4-sulfanyl-pentanoyl)amino]propanoate (0.0150 g, 1.92e-5 mol) in 3 mL of 2:1 CH₃CN/H₂O was added N-methylmorpholine (0.0600 mL, 0.000546 mol). The mixture was stirred for 36 h. LC-MS analysis indicated formation of the desired material. The mixture was purified by Gilson prep HPLC (Sunfire C18 30×150 mm; 20-80% CH₃CN/H₂O w/0.05% TFA; 16 min run; 13.5 min) to give desired conjugate. The mixture was purified by Gilson prep HPLC (Sunfire C18 30×150 mm; 20-72% CH₃CN/H₂O w/0.05% TFA; 15 min run; 12.5 min; retention time: 6.847 min) to give Compound 5 (0.0322 g, 7.94e-6 mol, yield: 53.8%). ESI (m/z=3): 1352.8.

### Example 5a: Alternative Synthesis of Compound 5

### Step 1. Preparation of Pv1-S-Pyridyl

Peptide Pv1 and 2,2′-dipyridyl disulfide were dissolved in MeOH and the reaction was stirred overnight. LC-MS indicated the desired product was formed. The reaction mixture was concentrated and the residue was taken up in DMSO and purified by reverse phase column chromatography (40-75% ACN/H₂O (0.5% AcOH), 15 min) to give 212 mg of the desired product.

### Step 2. Preparation of Compound 5

To a vial containing Pv1-SPyr (25.0 mg, 736e-6 mol) and [(1S,2R,3S,6S,16E,18E,20R,21S)-11-chloro-21-hydroxy12,20-dimethoxy-2,5,9,16-tetramethyl-8,23-dioxo-4,24-dioxa-9,22-diazatetracyclo[19.3.1.110,14.03,5]hexacosa10,12,14(26),16,18-pentaen-6-yl] (2S)-2-[methyl-(4-methyl-4-sulfanyl-pentanoyl)amino]propanoate (0.00864 g, 1.11e-5 mol). To this mixture was added 1 mL of degassed DMF and 0.5 mL degassed H₂O. CH₃CO₂H (0.017 mL, 0.000295 mol) was added. The mixture was stirred for 72 h. LC-MS indicated formation of the desired product. The mixture was purified by Gilson prep HPLC (Sunfire C18 30×150 mm; 20-80 CH₃CN/H₂O w/0.5% AcOH; 16 min run; 12.9 min) to give Compound 5 (0.00750 g, 1.85e-6 mol, yield: 25.1%).

### Example 10: Synthesis of Compound 10

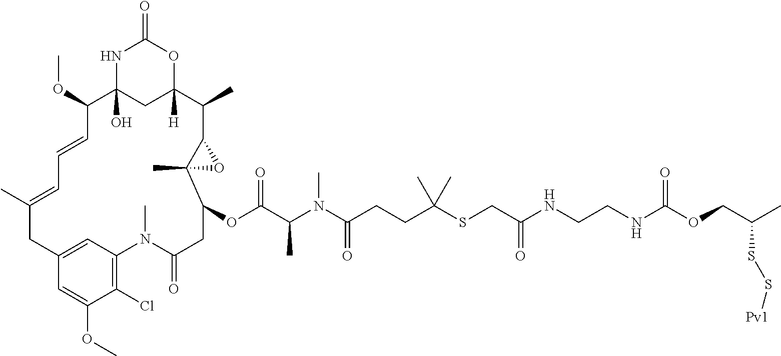

Step 1. (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(4-((2-((2,5-dioxopyrrolidin-1-yl)oxy)-2-oxoethyl)thio)-4-methylpentanoyl)-N-methyl-L-alaninate

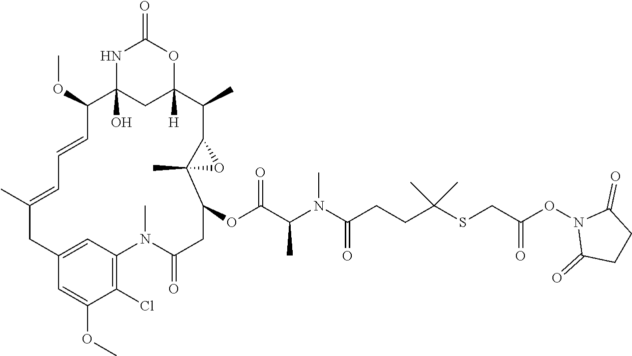

180 mg of DM4 (0.23 mmol) and 57 mg of bromoacetic acid N-hydroxysuccinimide ester (0.24 mmol) were dissolved in DMF (4.6 mL) and cooled in ice-water bath. 36.2 μL of DBU (0.24 mmol) was added at once and the mixture was allowed to warm to RT. At that moment LC/MS indicated nearly 95% conversion and the reaction was quenched with addition of 0.1 mL AcOH. Crude reaction mixture was directly loaded onto a 50 g C18 Aq column and purified via standard 10-100% B gradient (A: water w. 0.05% AcOH; B: water w. 0.05% AcOH). Product containing fractions were lyophilized to afford 160 mg of product (77% yield). HPLC purity at 254 nm: 96%. Retention time: 2.83 min (Method F). LCMS: 935.4 MH⁺.

### Step 2. 1⁴S,1⁶S,3²S,3³S,2R,4S,10E,12E,14R)-8⁶-chloro-1+-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(4-((2-((2-aminoethyl)amino)-2-oxoethyl)thio)-4-methylpentanoyl)-N-methyl-L-alaninate

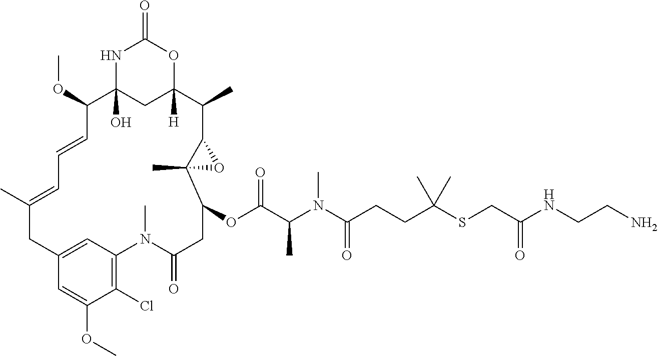

25 mg of 1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(4-((2-((2,5-dioxopyrrolidin-1-yl)oxy)-2-oxoethyl)thio)-4-methylpentanoyl)-N-methyl-L-alaninate (0.027 mmol) and 36 mg of N1-((4-methoxyphenyl)diphenylmethyl)ethane-1,2-diamine (0.11 mmol, 4 eq) were dissolved in dioxane (1 mL). After 3 h, the reaction appeared to be complete on LC/MS. The mixture was concentrated to dryness and dissolved in 80% AcOH in water (2 mL). LC/MS showed complete deprotection of the intermediate and the mixture was directly freeze-dried. The residue was dissolved in DMSO (1 mL) and loaded onto 15.5 g C18 Aq column and purified via standard 5-100% B gradient (A: water w. 0.05% AcOH; B: water w. 0.05% AcOH). Product containing fractions were lyophilized to afford 18 mg of product. HPLC purity at 254 nm: 95%. Retention time: 2.17 min (Method F). LCMS: 880.4 MH⁺.

### Step 3. (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (2S,18S)-2,3,7,7-tetramethyl-4,10,15-trioxo-18-(pyridin-2-yldisulfaneyl)-16-oxa-8-thia-3,11,14-triazanonadecanoate

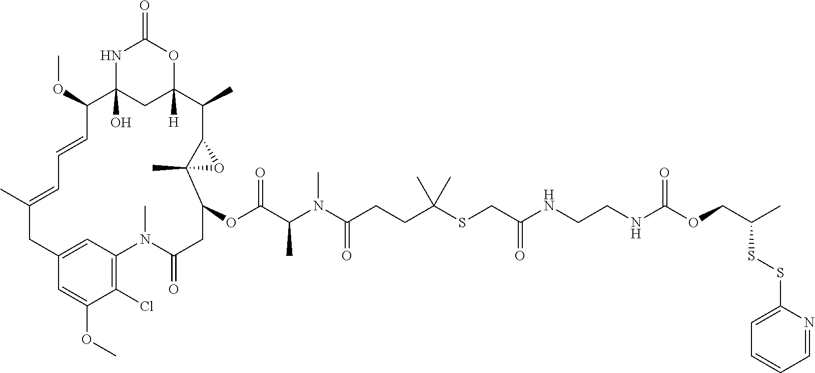

A solution of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(4-((2-((2-aminoethyl)amino)-2-oxoethyl)thio)-4-methylpentanoyl)-N-methyl-L-alaninate (14 mg, 0.016 mmol) in DMF (0.2 mL) was added to solid (S)-4-nitrophenyl (2-(pyridin-2-yldisulfaneyl)propyl) carbonate (6.6 mg, 0.018 mmol). Catalytic HOAt and DIEA (10 mL, 0.057 mmol) were added to the resultant solution and stirred at room temperature for 3 hours. The solution was neutralized with acetic acid (10 mL) and applied to a reverse phase column (RediSEP C18 (15.5 g)) and eluted with a gradient of acetonitrile (30% to 95%) in water with acetic acid (0.05%) to afford 18 mg (85% yield) of the title product. HPLC purity at 254 nm: 99%. Retention time: 2.85 min (Method F). LCMS: 1129.4 MNa⁺.

### Step 4. Synthesis of Compound 10

A solution of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (2S,18S)-2,3,7,7-tetramethyl-4,10,15-trioxo-18-(pyridin-2-yldisulfaneyl)-16-oxa-8-thia-3,11,14-triazanonadecanoate (17.7 mg, 0.00857 mmol) in DMF (1 mL) was treated with sodium bicarbonate (1.8 mg, 0.0214 mmol) and water (50 mL). The resultant solution was treated with peptide, Pv1 (31.5 mg, 0.0899 mmol) and stirred at room temperature for 3 hours, then applied to a reverse phase column, RediSep C18 (15.5 g) and eluted with a gradient of acetonitrile (30% to 70%) in water with ammonium acetate (10 mM). The fractions were combined, frozen and lyophilized to afford the product as a white solid, 18.7 mg (50%). HPLC purity at 254 nm: 99%. Retention time: 6.49 min (Method G) LCMS: 2138.0 (M+2H)/2⁺, 1425.3 (M+3H)/3⁺.

### Example 11: Synthesis of Compound 11

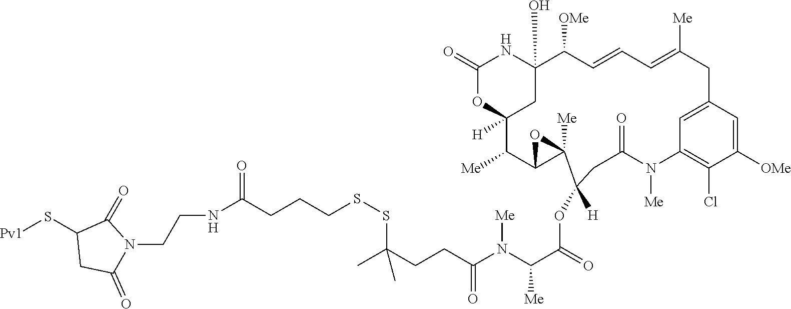

Step 1. (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(4-((4-((2-(2,5-dioxo-2,5-dihydro-1H-pyrrol-1-yl)ethyl)amino)-4-oxobutyl)disulfaneyl)-4-methylpentanoyl)-N-methyl-L-alaninate

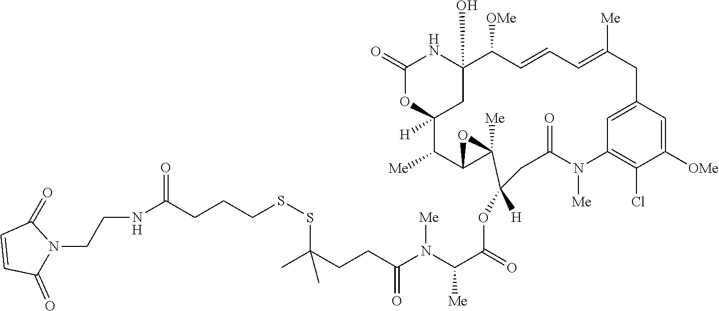 [SMILES: COc1cc2cc(c1Cl)N(C)C(=O)C[C@H](OC(=O)[C@H](C)N(C)C(=O)CCC(C)(C)SSCCCC(=O)NCCN1C(=O)C=CC1=O)[C@]1(C)O[C@H]1[C@H](C)[C@@H]1C[C@@](O)(NC(=O)O1)[C@H](OC)/C=C/C=C(\C)C2]

DM4 (10 mg, 0.013 mmol) and succinimidyl 4-(2-pyridyldithio)butanoate (6 mg, 0.02 mmol) were mixed in DMF (0.26 mL). Triethylamine was added (0.015 mL) and the mixture was stirred for 2 h. 1-(2-aminoethyl)-1H-pyrrole-2,5-dione hydrochloride (5 mg, 0.026 mmol) was added and after 3 h the mixture was directly loaded onto a RediSEP C18 Aq (15.5 g) column and eluted with a gradient of acetonitrile (30% to 95%) in water with acetic acid (0.05%) to afford 6 mg (40% yield) of the title product. HPLC purity at 254 nm: 92%. Retention time: 2.83 min (Method F). LCMS: 1020.4 MH⁺.

### Step 2. Synthesis of Compound 11

A solution of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(4-((4-((2-(2,5-dioxo-2,5-dihydro-1H-pyrrol-1-yl)ethyl)amino)-4-oxobutyl)disulfaneyl)-4-methylpentanoyl)-N-methyl-L-alaninate (6 mg, 0.006 mmol) and Pv1 peptide (22.3 mg, 0.006 mmol) were dissolved in DMF (0.12 mL) and treated with triethylamine (0.001 mL). After 30 minutes the reaction mixture was directly loaded onto a RediSEP C8 (15.5 g) column and eluted with a gradient of acetonitrile (35% to 75%) in water with TFA (0.05%) to afford 16 mg (64% yield) of the title compound. HPLC purity at 254 nm: 98%. Retention time: 6.19 min (Method G). LCMS: 2150.2 (M+2H)/2⁺, 1433.3 (M+3H)/3⁺.

### Example 12. Synthesis of Compound 12

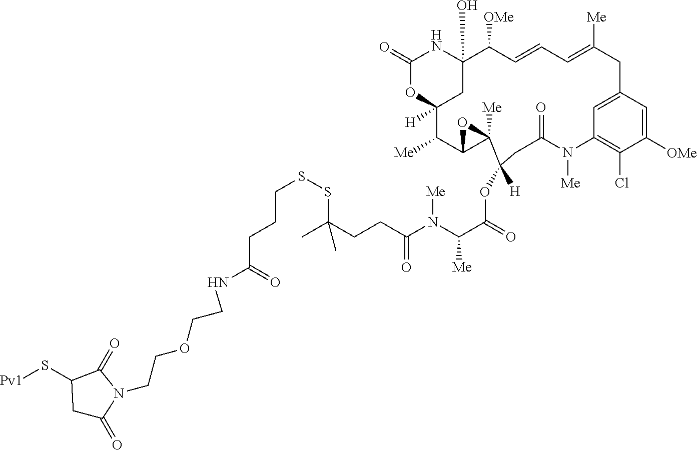

### Step 1. ((1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-1-(2,5-dioxo-2,5-dihydro-1H-pyrrol-1-yl)-16,16,20,21-tetramethyl-10,19-dioxo-3,6-dioxa-14,15-dithia-9,20-diazadocosan-22-oate

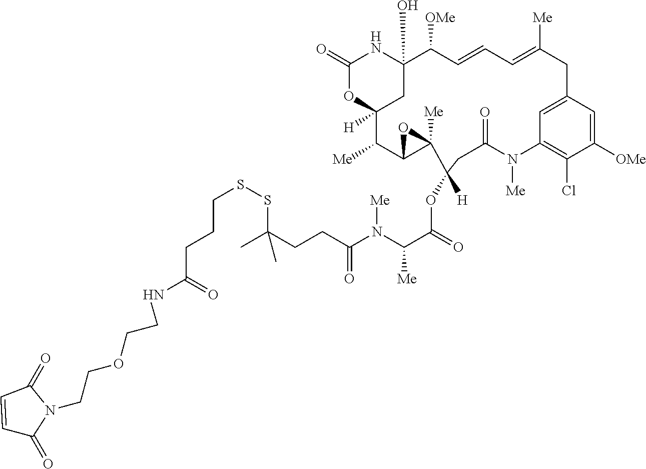 [SMILES: COc1cc2cc(c1Cl)N(C)C(=O)C[C@H](OC(=O)[C@H](C)N(C)C(=O)CCC(C)(C)SSCCCC(=O)NCCOCCN1C(=O)C=CC1=O)[C@]1(C)O[C@H]1[C@H](C)[C@@H]1C[C@@](O)(NC(=O)O1)[C@H](OC)/C=C/C=C(\C)C2]

DM4 (20 mg, 0.026 mmol) and succinimidyl 4-(2-pyridyldithio)butanoate (12 mg, 0.04 mmol) were mixed in DMF (0.75 mL). Triethylamine was added (0.045 mL) and the mixture was stirred for 2 h. 1-(21-(2-(2-(2-aminoethoxy)ethoxy)ethyl)-1H-pyrrole-2,5-dione hydrochloride (9 mg, 0.036 mmol) was added and after 3 h the mixture was directly loaded onto a RediSEP C18 Aq (15.5 g) column and eluted with a gradient of acetonitrile (30% to 95%) in water with acetic acid (0.05%) to afford 17 mg (61% yield). HPLC purity at 254 nm: 99%. Retention time: 2.84 min (Method F). LCMS: 1108.4 MH⁺.

### Step 2. Synthesis of Compound 12

A solution of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-1-(2,5-dioxo-2,5-dihydro-1H-pyrrol-1-yl)-16,16,20,21-tetramethyl-10,19-dioxo-3,6-dioxa-14,15-dithia-9,20-diazadocosan-22-oate (15 mg, 0.014 mmol) and Pv1 peptide (52 mg, 0.015 mmol) were dissolved in DMF (0.28 mL) and treated with triethylamine (0.006 mL). After 30 minutes the reaction mixture was directly loaded onto a RediSEP C8 (15.5 g) column and eluted with a gradient of acetonitrile (35% to 60%) in water with TFA (0.05%) to afford 25 mg (34% yield). HPLC purity at 254 nm: 98%. Retention time: 6.26 min (Method G). LCMS: 2194.0 (M+2H)/2⁺, 1463.0 (M+3H)/3⁺.

### Example 13. Synthesis of Compound 13

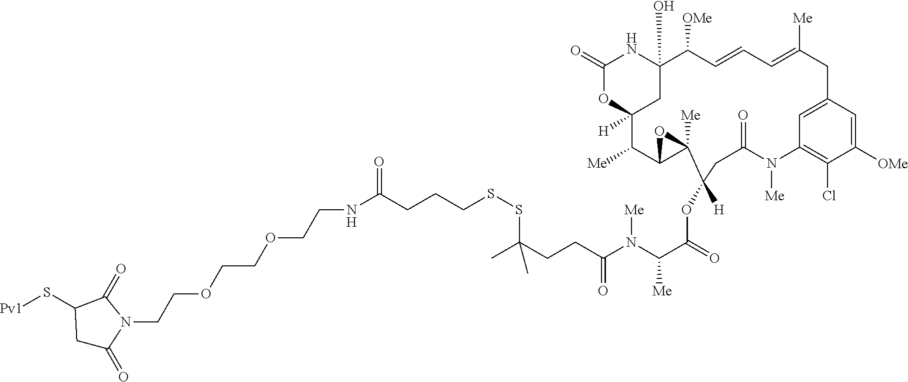

### Step 1. 1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-1-(2,5-dioxo-2,5-dihydro-1H-pyrrol-1-yl)-13,13,17,18-tetramethyl-7,16-dioxo-3-oxa-11,12-dithia-6,17-diazanonadecan-19-oate

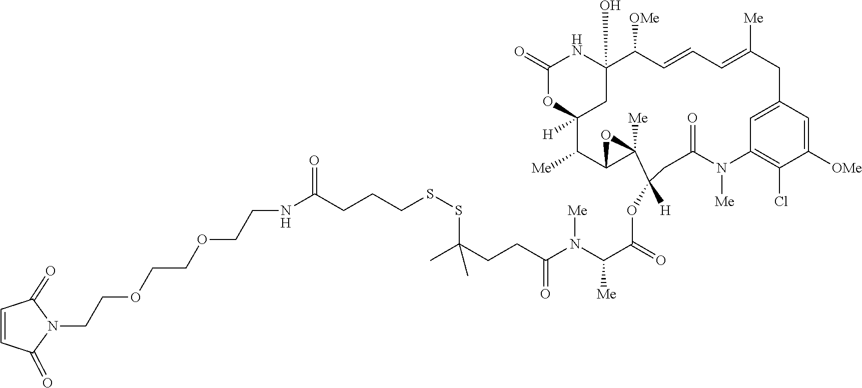 [SMILES: COc1cc2cc(c1Cl)N(C)C(=O)C[C@H](OC(=O)[C@H](C)N(C)C(=O)CCC(C)(C)SSCCCC(=O)NCCOCCOCCN1C(=O)C=CC1=O)[C@]1(C)O[C@H]1[C@H](C)[C@@H]1C[C@@](O)(NC(=O)O1)[C@H](OC)/C=C/C=C(\C)C2]

DM4 (20 mg, 0.026 mmol) and succinimidyl 4-(2-pyridyldithio)butanoate (12 mg, 0.04 mmol) were mixed in DMF (0.75 mL). Triethylamine was added (0.045 mL) and the mixture was stirred for 2 h. 1-(2-(2-aminoethoxy)ethyl)-1H-pyrrole-2,5-dione hydrochloride (5 mg, 0.024 mmol) was added and after 3 h the mixture was directly loaded onto a RediSEP C18 Aq (15.5 g) column and eluted with a gradient of acetonitrile (30% to 95%) in water with acetic acid (0.05%) to afford 13 mg (41% yield) of the title product. HPLC purity at 254 nm: 94%. Retention time: 2.85 min (Method F). LCMS: 1064.4 MH⁺.

### Step 2. Synthesis of Compound 13

A solution of (14S,16S,32S,33S,2R,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl (S)-1-(2,5-dioxo-2,5-dihydro-1H-pyrrol-1-yl)-16,16,20,21-tetramethyl-10,19-dioxo-3,6-dioxa-14,15-dithia-9,20-diazadocosan-22-oate (18 mg, 0.017 mmol) and Pv1 peptide (63 mg, 0.019 mmol) were dissolved in DMF (0.34 mL) and treated with triethylamine (0.007 mL). After 30 minutes the reaction mixture was directly loaded onto a RediSEP C8 (15.5 g) column and eluted with a gradient of acetonitrile (35% to 60%) in water with TFA (0.05%) to afford 25 mg (34% yield) of the title compound. HPLC purity at 254 nm: 99%. Retention time: 6.24 min (Method G). LCMS: 2172.0 (M+2H)/2⁺, 1448.7 (M+3H)/3⁺.

### Example 14. Synthesis of Compound 14

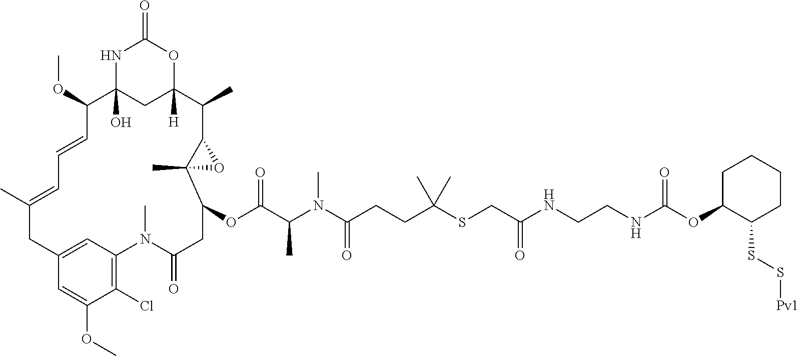

Step 1. (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-9,9,13,14-tetramethyl-1,6,12-trioxo-1-(((1S,2S)-2-(pyridin-2-yldisulfaneyl)cyclohexyl)oxy)-8-thia-2,5,13-triazapentadecan-5-oate

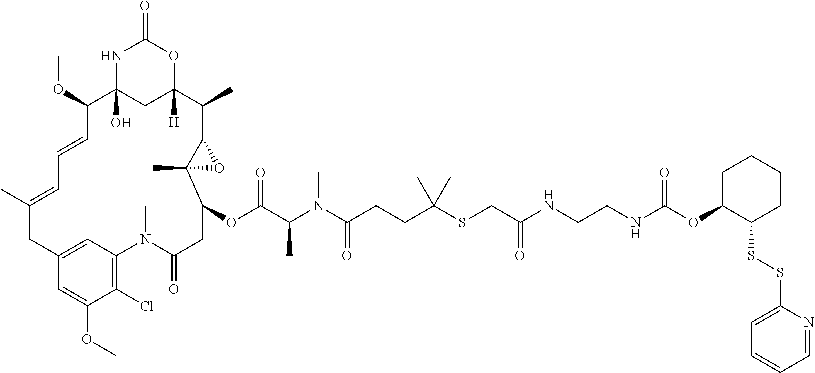

A solution of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(4-((2-((2-aminoethyl)amino)-2-oxoethyl)thio)-4-methylpentanoyl)-N-methyl-L-alaninate (14 mg, 0.016 mmol; Example 10, Step 2) in DMF (0.2 mL) was added to solid 4-nitrophenyl ((1S,2S)-2-(pyridin-2-yldisulfaneyl)cyclohexyl) carbonate (7.2 mg, 0.018 mmol). Catalytic HOAt and DIEA (10 mL, 0.057 mmol) were added to the resultant solution and stirred at room temperature for 3 hours. The solution was neutralized with acetic acid (10 mL) and applied to a reverse phase column, RediSEP C18 (15.5 g) and eluted with a gradient of acetonitrile (30% to 95%) in water with acetic acid (0.05%) to afford 15 mg (80% yield) of the title compound. HPLC purity at 254 nm: 98%. Retention time: 3.05 min (Method F). LCMS: 1147.4 MH⁺.

### Step 2. Synthesis of Compound 14

A solution of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-9,9,13,14-tetramethyl-1,6,12-trioxo-1-(((1S,2S)-2-(pyridin-2-yldisulfaneyl)cyclohexyl)oxy)-8-thia-2,5,13-triazapentadecan-15-oate (17 mg, 0.015 mmol) and Pv1 peptide (47 mg, 0.013 mmol) were dissolved in DMF (0.34 mL) and treated with triethylamine (0.007 mL). After 30 minutes the reaction mixture was directly loaded onto a RediSEP C8 (15.5 g) column and eluted with a gradient of acetonitrile (35% to 60%) in water with TFA (0.05%) to afford 28 mg (37% yield). HPLC purity at 254 nm: 99%. Retention time: 7.36 min (Method G). LCMS: 2158.0 (M+2H)/2⁺, 1439.0 (M+3H)/3⁺.

### Example 15. Synthesis of Compound 15

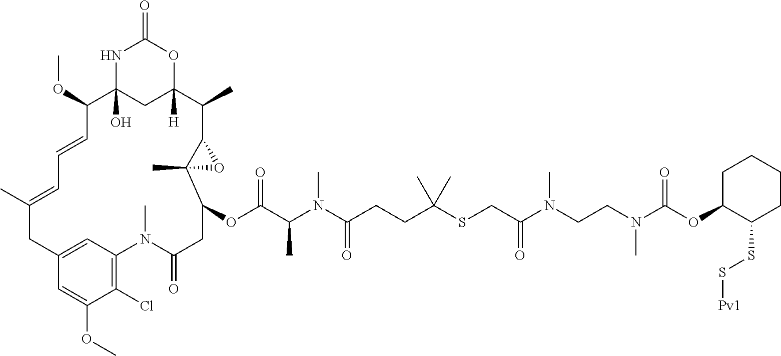

Step 1. (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-5,9,9,13,14-pentamethyl-6,12-dioxo-8-thia-2,5,13-triazapentadecan-15-oate

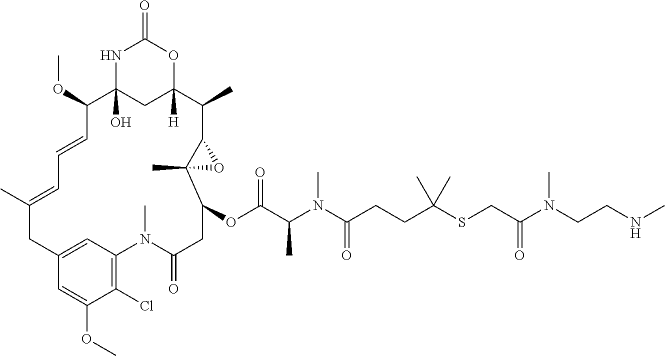

25 mg of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(4-((2-((2,5-dioxopyrrolidin-1-yl)oxy)-2-oxoethyl)thio)-4-methylpentanoyl)-N-methyl-L-alaninate (0.027 mmol) and 40 mg of N1-((3-methoxyphenyl)diphenylmethyl)-N1,N2-dimethylethane-1,2-diamine (0.11 mmol, 4 eq) were dissolved in dioxane (1 mL). After 3 h, the reaction appeared to be complete on LC/MS. 0.05 mL of TFA was added and the mixture was loaded onto a 15.5 g C18 Aq column and purified via standard 5-100% B gradient (A: water w. 0.05% TFA; B: ACN w. 0.05% TFA). Product containing fractions were lyophilized to afford 22 mg of product (75% yield). HPLC purity at 254 nm: 98%. Retention time: 2.25 min (Method F). LCMS: 908.4 MH⁺.

### Step 2. (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-2,5,9,9,13,14-hexamethyl-1,6,12-trioxo-1-(((i S,2S)-2-(pyridin-2-yldisulfaneyl)cyclohexyl)oxy)-8-thia-2,5,13-triazapentadecan-15-oate

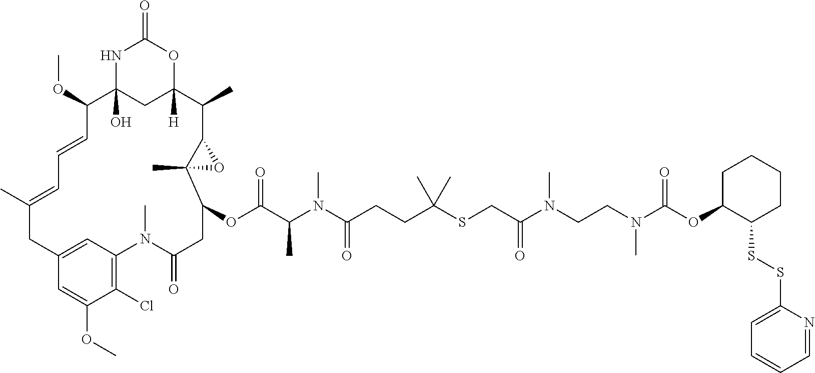

A solution of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-5,9,9,13,14-pentamethyl-6,12-dioxo-8-thia-2,5,13-triazapentadecan-15-oate (15 mg, 0.016 mmol) in DMF (0.2 mL) was added to solid 4-nitrophenyl ((1S,2S)-2-(pyridin-2-yldisulfaneyl)cyclohexyl) carbonate (6.6 mg, 0.018 mmol). Catalytic HOAt and DIEA (10 mL, 0.057 mmol) were added to the resultant solution and stirred at room temperature for 3 hours. The solution was neutralized with acetic acid (10 mL) and applied to a reverse phase column, RediSEP C18 (15.5 g) and eluted with a gradient of acetonitrile (30% to 95%) in water with acetic acid (0.05%) to afford 16 mg (82% yield) of the title product. HPLC purity at 254 nm: 97%. Retention time: 3.36 min (Method F). LCMS: 1175.5 MH⁺.

### Step 3. Synthesis of Compound 15

A solution of (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-2,5,9,9,13,14-hexamethyl-1,6,12-trioxo-1-(((1S,2S)-2-(pyridin-2-yldisulfaneyl)cyclohexyl)oxy)-8-thia-2,5,13-triazapentadecan-15-oate (16 mg, 0.014 mmol) and Pv1 peptide (52 mg, 0.015 mmol) were dissolved in DMF (0.28 mL) and treated with triethylamine (0.008 mL). After 30 minutes the reaction mixture was directly loaded onto a RediSEP C8 (15.5 g) column and eluted with a gradient of acetonitrile (35% to 60%) in water with TFA (0.05%) to afford 32 mg (44% yield) of the title compound. HPLC purity at 254 nm: 99%. Retention time: 6.86 min (Method G). LCMS: 2172.0 (M+2H)/2⁺, 1448.4 (M+3H)/3⁺.

### Example 16. Synthesis of Compound 16

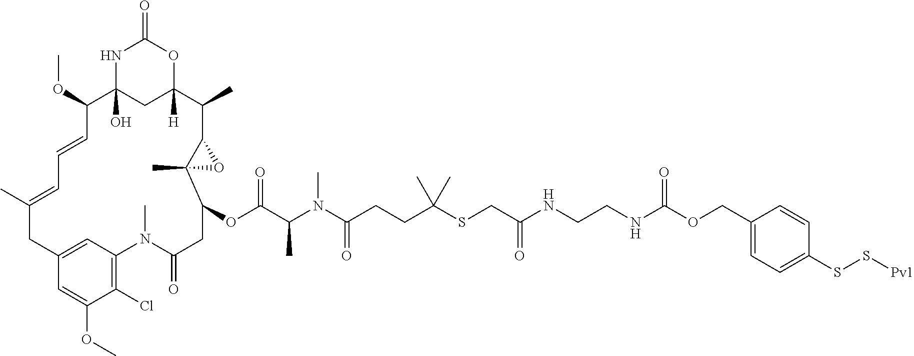

### Step 1. (4-((5-nitropyridin-2-yl)disulfaneyl)phenyl)methanol

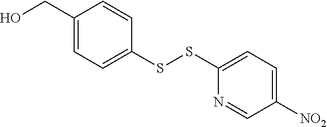 [SMILES: O=[N+]([O-])c1ccc(SSc2ccc(CO)cc2)nc1]

A solution of (4-mercaptophenyl)methanol (0.74 g, 4.83 mmol) in THE (10 mL) was treated with 5-nitro-2-((4-nitrophenyl)disulfaneyl)pyridine (1.0 g, 3.23 mmol). The resultant suspension was stirred at room temperature for 2 hours, and the solvent was evaporated in vacuo. The residue was dissolved in DCM and applied to a RediSep silica gel column and eluted with a gradient of ethyl acetate (10% to 60%) in hexanes to afford the product (0.499 g, 52% yield). HPLC purity at 254 nm: 90%. Retention time: 2.72 min (Method F). MS data, 295.1 (M+H)⁺. ¹HNMR (DMSO-d₆) δ 9.18 (s, 1H), 8.58 (d of d, 1H), 8.02 (d, 1H), 7.56 (d, 2H), 7.34 (d, 2H), 5.24 (t, 1H) and 4.47 (d, 2H).

### Step 2. 4-nitrophenyl (4-((5-nitropyridin-2-yl)disulfaneyl)benzyl) carbonate

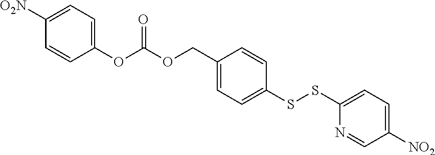 [SMILES: O=C(OCc1ccc(SSc2ccc([N+](=O)[O-])cn2)cc1)Oc1ccc([N+](=O)[O-])cc1]

A solution of 4-nitrophenyl chloroformate (255 mg, 1.26 mmol) in THE (5 mL) was cooled on an ice-bath and treated with a solution (4-((5-nitropyridin-2-yl)disulfaneyl)phenyl)methanol (220 mg, 0.748 mmol), triethyl amine (0.7 mL, 5.03 mmol), and 4-dimethyaminopyridine (45 mg, 0.368 mmol) in THE (5 mL) added over about 15 minutes. The ice-bath was removed, and the solution was stirred at room temperature for one hour and stored in a freezer overnight. The solvent was evaporated in vacuo, and the residue was dissolved in DCM, applied to a RediSep silica gel column (12 g) and eluted with a gradient of ethyl acetate (2% to 100%) in hexanes. The product was purified further by reverse phase chromatography on a RediSep C18 cartridge (50 g) eluted with a gradient of acetonitrile (30% to 95%) in water with acetic acid (0.05%), to afford the product, 55 mg (16%). HPLC purity at 254 nm: >99%. Retention time: 3.77 min (Method F). MS data, 460.7 (M+H)⁺. ¹HNMR(CDCl₃) δ 9.29 (d, 1H), 8.39 (d of d, 1H), 8.28 (d of d, 2H), 7.83 (d of d, 1H), 7.55 (d of d, 2H), 7.44 (d of d), 7.36 (d of d, 2H) and 5.26 (d, 2H).

### Step 3. (14S,16S,32S,33S,2R,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl (S)-11,11,15,16-tetramethyl-1-(4-((5-nitropyridin-2-yl)disulfaneyl)phenyl)-3,8,14-trioxo-2-oxa-10-thia-4,7,15-triazaheptadecan-17-oate

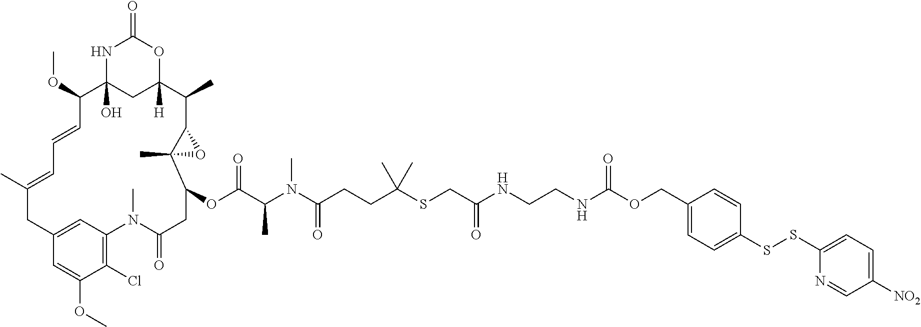

A solution of (14S,16S,32S,33S,2R,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl N-(4-((2-((2-aminoethyl)amino)-2-oxoethyl)thio)-4-methylpentanoyl)-N-methyl-L-alaninate (15 mg, 0.017 mmol; Example 10, Step 2) in DMF (1 mL) was added to solid 4-nitrophenyl (4-((5-nitropyridin-2-yl)disulfaneyl)benzyl) carbonate (26 mg, 0.0566 mmol). Catalytic HOAt and DIEA (10 mL, 0.057 mmol) were added to the resultant solution and stirred at room temperature for 3 hours. The solution was neutralized with acetic acid (7 mL, 0.122 mmol) and applied to a reverse phase column, RediSEP C18 (15.5 g) and eluted with a gradient of acetonitrile (30% to 95%) in water with acetic acid (0.05%). Further purification on a silica gel column, RediSep (4 g), using a gradient of methanol (0.2% to 6%) in DCM as the eluant, afforded the title product (10.3 mg, 50% yield). HPLC purity at 254 nm: >99%. Retention time: 3.16 min (Method F). MS data, 1182.3 (M+H−H₂O)⁺, 1201.3 (M+H)⁺, 1222.3 (M+Na)⁺.

### Step 4. Synthesis of Compound 16

A solution of (14S,16S,32S,33S,2R,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl (S)-11,11,15,16-tetramethyl-1-(4-((5-nitropyridin-2-yl)disulfaneyl)phenyl)-3,8,14-trioxo-2-oxa-10-thia-4,7,15-triazaheptadecan-17-oate (10.3 mg, 0.00857 mmol) in DMF (1 mL) was treated with sodium bicarbonate (1.8 mg, 0.0214 mmol) and water (50 mL). The resultant solution was treated with peptide, Pv1 (31.5 mg, 0.0899 mmol) and stirred at room temperature for 3 hours, then applied to a reverse phase column, RediSep C18 (15.5 g) and eluted with a gradient of acetonitrile (30% to 70%) in water with ammonium acetate (10 mM). The fractions were combined, frozen and lyophilized to afford the product as a white solid, 18.7 mg (50%). HPLC purity at 254 nm: 99%. Retention time: 6.63 min (Method G). MS data, 2162.4 (M+2H)/2⁺, 1441.8 (M+3H)/3⁺, 1082.6 (M+4H)/4⁺, 1435.7 (M+3H−H₂O)/3⁺.

### Example A. Growth Delay Assay

Cells were plated in 96 well black walled-clear bottom plates (Griener), DLD-1 WT cells at 2500 cells per well, FaDu, and HeLa cells at 5000 cells per well, and HCT116 at 3000 cells per well, in growth media containing 1000 FBS. Cells were allowed to adhere at room temperature for 60 minutes before returning to a 37 C, 5% CO₂ incubator. After 24 hours, media was removed and replaced with fresh growth media containing various drug concentrations. Each drug concentration was added in triplicate. Non-drug treated controls contained growth media only. Cells were returned to the incubator. Ninety-six hours after addition of drug, cells were fixed with 40% paraformaldehyde for 20 minutes and stained with Hoechst at 1 μg/mL. The plates were imaged on a Cytation 5 auto imager (BioTek) and cells were counted using CellProfiler (http://cellprofiler.org). The percent cell growth delay was calculated and data plotted using GraphPad Prism

TABLE 5
Growth Delay Assay data

| | DLD-1 | HCT116 | FaDu | HeLa |
|---|---|---|---|---|
| Example | (IC₅₀, nM) | (IC₅₀, nM) | (IC₅₀, nM) | (IC₅₀, nM) |
| R²SH-1 | 9.8 | 4.3 | 2.8 | 2.6 |
| (see Table 2) | | | | |
| R²SH-2 | 0.45 | 0.20 | 0.13 | 0.02 |
| (see Table 2) | | | | |
| 1 | 60.5 | 21.8 | 10.4 | 7.4 |
| 2 | 114 | 21.1 | 15.4 | 8.0 |
| 3 | 45.1 | 19.3 | 10.1 | 6.9 |
| 4 | 11.7 | 2.5 | 1.4 | 0.85 |
| 5 | 9.4 | 2.7 | 2.3 | 0.95 |
| 6 | 8.3 | 3.3 | 5.2 | 1.8 |
| 7 | NC* | 3.4 | 4.6 | 2.0 |
| 8 | 9.8 | 3.0 | 3.4 | 1.6 |

NC* = Not calculated

### Example B: Effect on In Vitro Tubulin Polymerization

A fluorescence-based tubulin polymerization assay (Cytoskeleton Cat #BK011P) was performed to quantitate the impact of unconjugated DM4 and Compound 5 on in vitro tubulin polymerization. DM4 and Compound 5 were prepared as 10 mM stocks in DMSO then diluted at 10× to 200, 50 and 5 μM in ultrapure distilled water for a final DMSO concentration of 0.2%. Kit reagents were defrosted rapidly then kept cold on ice to prevent premature polymerization. A tubulin reaction mixture was prepared on ice by mixing purified porcine brain tubulin, GTP, and glycerol buffer all in 1× kit buffer for final concentrations of 2 mg/mL tubulin, 1 mM GTP and 15% glycerol. 5 μL of DM4, Compound 5 or DMSO control were added to a pre-warmed black, half-well reaction plate at 37° C. for no longer than 1 minute, to warm, but not allow for evaporation. 50 μL of tubulin reaction mixture was rapidly added to each well and immediately placed in a pre-warmed Cytation 5 imaging reader (BioTek). A kinetic reading was performed at 360 excitation/450 emission for 2 hours at 37° C., with readings every 2.5 minutes to follow the enhancement of fluorescence due to incorporation of a fluorescent reporter into microtubules as polymerization occurs.

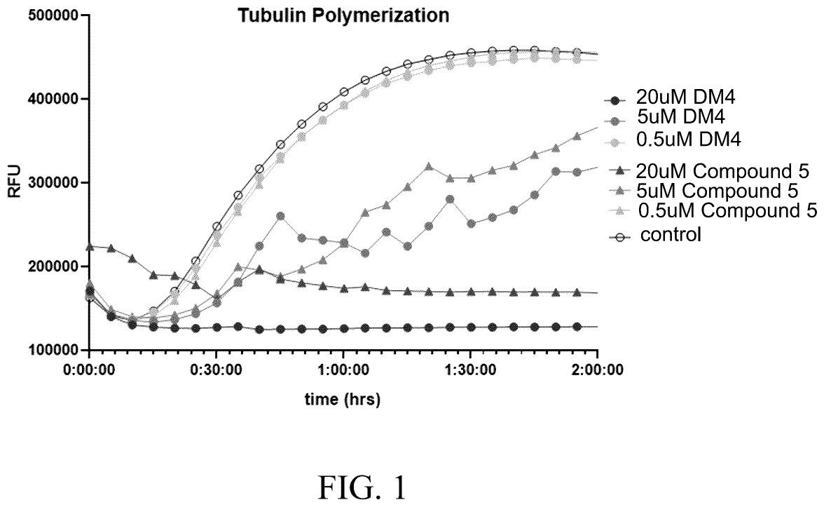

FIG. 1 shows a plot of the effect of free DM4 and Compound 5 on in vitro β-tubulin polymerization (in terms of relative fluorescence units) at 0.5 μM, 5 μM, and 20 μM.

### Example C: Kinetic Analysis of Conjugate Binding

Binding experiments were performed using a Biacore S200 instrument. A Series S sensor chip with pre-immobilized streptavidin was conditioned with 1 M NaCl in 50 mM NaOH. Biotin-labeled human tubulin derived from HeLa cells was immobilized to the sensor chip at a concentration of 125 μg/mL in HBS-P+ buffer at a flow rate of 10 μl/min. A final 3000 RU (response units) of protein was directly immobilized to the chip. After tubulin immobilization, the sensor chip was washed with 50% isopropanol, 50 mM NaOH and 1 M NaCl and subsequently allowed to equilibrate in assay buffer for 4 hours. A streptavidin-biotin capture blank (reference FC) was used to monitor non-specific binding.

To collect kinetic binding data, Compound 5 diluted in assay buffer was injected over the flow cells at concentrations ranging from 100 μM to 0.048 μM and 50 μM to 0.024 μM, at a flow rate of 60 μL/minute and a temperature of 25° C. The complex was allowed to dissociate for 60 seconds. Binding of compound to tubulin was monitored in real time to obtain on (Ka) and off (Koff) rates. The affinity constant (KD) was calculated by steady state kinetics.

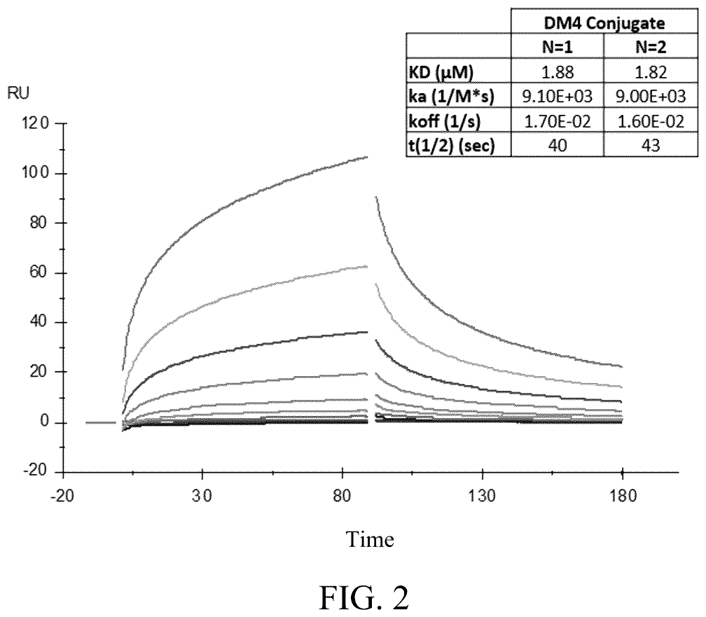

FIG. 2 depicts the kinetic analysis of Compound 5 binding to β-tubulin in vitro as determined by Biacore surface plasmon resonance. Compound 5 is able to bind to β-tubulin with a similar KD as free DM4 (3.55 μM) and slower on/off rates relative to free DM4.

### Example D: Efficacy of Compound 5 in a Mouse Colorectal Cancer Model

Six-week-old female athymic nude Foxn^{nu} mice were obtained from Taconic Labs (Cat #NCRNU-F) and were housed 5 per cage on Alpha-Dri bedding in a disposable caging system. Human HCT116 cells derived from colorectal carcinoma were diluted 1:1 in Phenol Red-free Matrigel and subcutaneously implanted into the left flank of each mouse at a density of 2.5×10⁶ cells in 100 μL. When xenografts reached a mean volume of 100-200 mm³, mice were randomized into groups and treated as detailed in the table below. Mice were administered intraperitoneal (IP) doses of vehicle or 0.21, 0.29, 0.35, 0.42 μmole/kg Compound 5 (equivalent to 1.1, 1.4, 1.7, or 2 mg/kg Compound 5) or 0.42 μmole/kg unconjugated DM4 (equivalent to 0.33 mg/kg unconjugated DM4). Doses were prepared by diluting 0.1 mg/μL DMSO stocks in 5% mannitol in citrate buffer and were administered QDX4 with a two day interval between the second and third doses, at a volume of 12 mL/kg (300 μL per 25 g mouse). Xenograft tumors were measured by calipers and volume was calculated using the equation for ellipsoid volume: Volume=π/6×(length)×(width)². Body weight of animals was measured at the same time as tumor volume assessment. Animals were removed from the study due to death, tumor size exceeding 2000 mm³ or loss of >20% body weight. Kaplan-Meier analysis was used to evaluate survival rate based on death or removal from study.

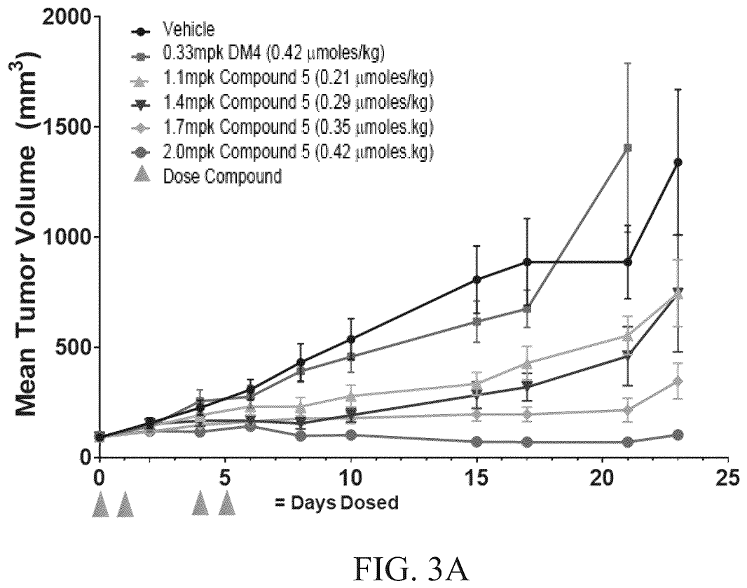

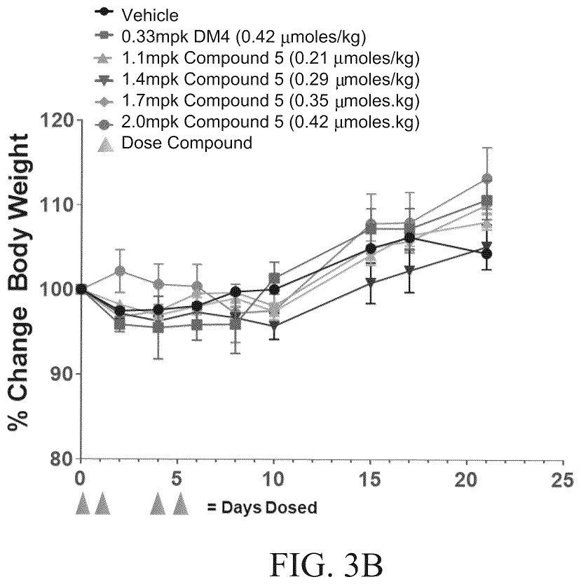

FIG. 3A shows a plot of the mean tumor volume in nude mice bearing HCT116 colorectal flank tumors dosed with DM4 or Compound 5.

FIG. 3B shows the percent change in body weight of nude mice bearing HCT116 colorectal flank tumors dosed with DM4 or Compound 5 relative to day 0.

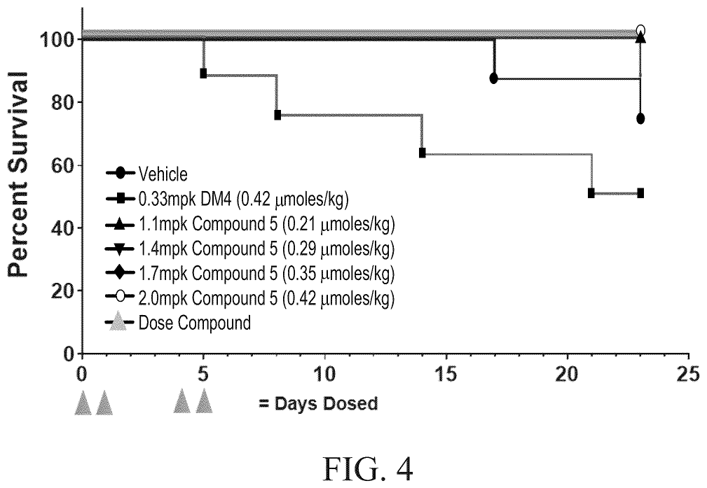

FIG. 4 depicts a Kaplan-Meier plot of nude mice bearing HCT116 colorectal flank tumors dosed with DM4 or Compound 5. Animals were removed from the study due to either death, tumor size exceeding 2000 mm³ or due to loss of greater than 20% body weight. Free DM4 induced the spontaneous death of half of the DM4 group of animals during the post-dosing period. As shown in FIG. 4, Compound 5 safely delivers amounts of DM4 in vivo that otherwise result in systemic toxicity and death when dosed as free DM4.

### Example E: Effect of Compound 6 on Lung Metastases in a Mouse Lung Cancer Model

Mouse 4T1-iRFP cancer cells derived from mouse mammary carcinoma and transfected with near infrared fluorescent protein (iRFP) were cultured as a monolayer at 37° C. in a humidified atmosphere with 5% CO₂. Cells were passaged between one and three days prior to implantation and media was replaced every 2-3 days as needed to maintain cell viability. Cells were not allowed to exceed 80% confluency. On the day of implantation, cells were trypsinized, washed with complete media and pelleted by centrifugation at 1200 rpm for 5 minutes. The supernatant was decanted, and cells were washed three times with sterile PBS and pelleted by centrifugation. During the final centrifugation, viability was determined using trypan blue exclusion. Cells were resuspended in sterile PBS a final concentration of 5×10⁵ cells/100 μL. Cells were drawn into sterile 1 cc tuberculin syringes with a 27-gauge needle. Air bubbles were removed, and excess cell mixture was expelled back into the conical tube leaving an injection volume of 100 μL in each syringe. The 100 μL of cells were injected directly into the medial tail vein of six-week-old female athymic nude Foxn^{nu} mice (Taconic Labs Cat #NCRNU-F).

Three days after cell injection, mice were administered intraperitoneal doses of vehicle or 2.5 mg/kg Compound 6 once daily for 2 days followed by 2 days of no treatment, followed by a single dose of Compound 6, for a total of three total doses of Compound 6. Eleven days after injection, mice were euthanized, and the lungs were removed for imaging using the LI-COR PEARL Trilogy small animal imager to visualize and quantitate lung metastasis and evaluate compound effect on tumor growth.

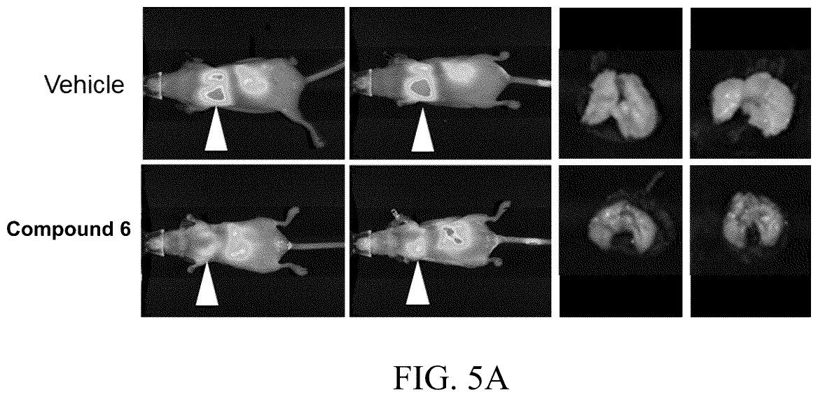

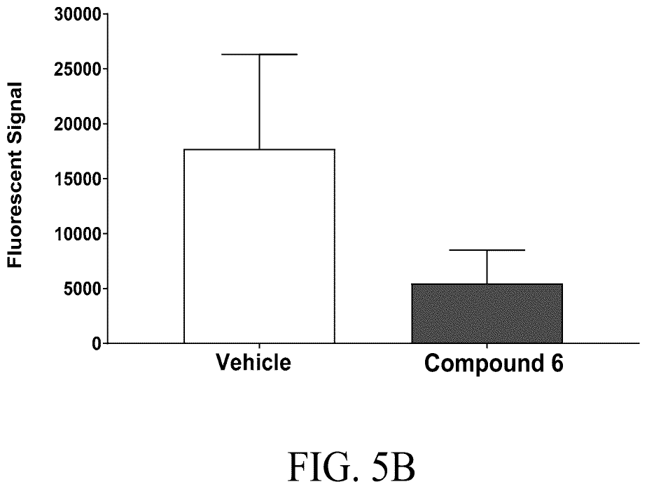

FIG. 5A depicts the ventral view and extracted lungs of nude mice inoculated with 4T1-RFP fluorescent cells via tail vein injection and imaged 11 days after inoculation and after 3 doses of vehicle or Compound 6.

FIG. 5B depicts a graph of the fluorescent signal from extracted lungs of 4T1-RFP inoculated mice after 3 doses of vehicle or Compound 6.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1-29. (canceled)

30. A method of treating cancer in a patient in need thereof comprising administering to the patient a therapeutically effective amount of a compound of ci Formula (I):
R²-L-R¹ (I)
or a pharmaceutically acceptable salt thereof, wherein:
R¹ is a peptide capable of selectively delivering R²L- across a cell membrane having an acidic or hypoxic mantle;
R² is selected from the group consisting of: 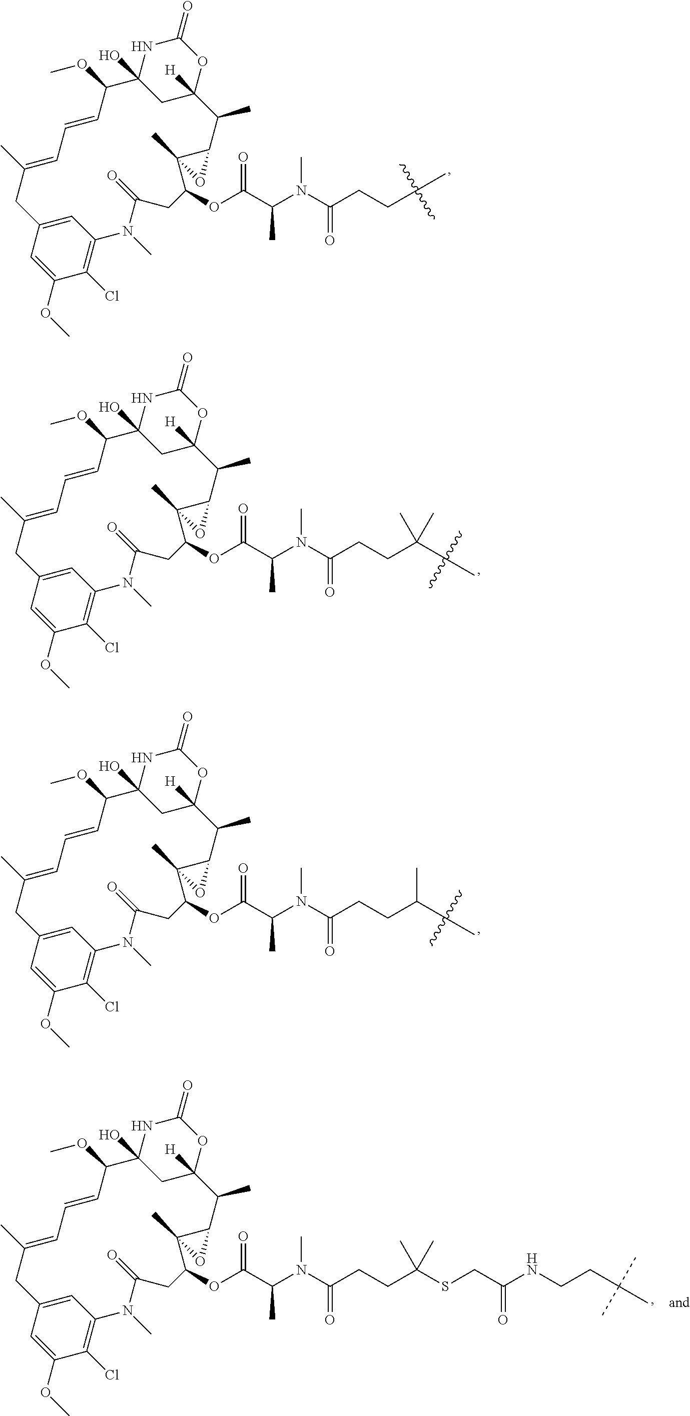 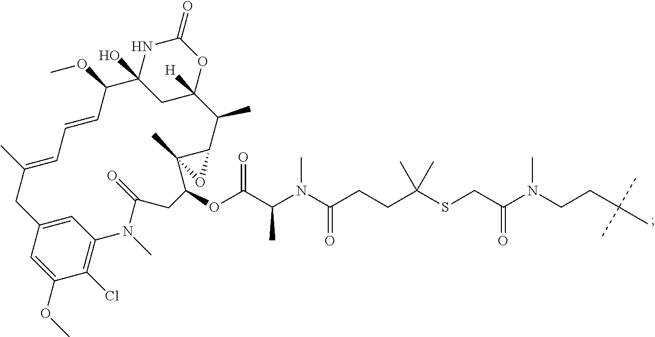
L is the following group: 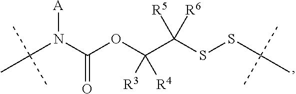
wherein
R³, R⁴, R⁵, and R⁶ are each independently selected from H, C₁₋₄ alkyl, C₁₋₄ alkenyl, C₆₋₁₀ aryl, C₃₋₁₀ cycloalkyl, 5-10 membered heteroaryl, 4-10 membered heterocycloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1}, wherein said C₁₋₄ alkyl, C₁₋₄ alkenyl, C₆₋₁₀ aryl, and 5-10 membered heteroaryl are each optionally substituted with 1, 2, or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R³ and R⁴ together with the carbon atom to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R³ and R⁵ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁴ and R⁶ together with the carbon atoms to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
or R⁵ and R⁶ together with the carbon atom to which they are attached form a C₃₋₁₄ cycloalkyl group or 4-14 membered heterocycloalkyl group, each optionally substituted with 1, 2, or 3 substituents independently selected from C₁₋₄ alkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, and NR^{c1}C(O)NR^{c1}R^{d1};
A is H or C₁₋₄ alkyl; and
R^{a1}, R^{b1}, R^{c1} and R^{d1} are each independently selected from H, C₁₋₆ alkyl, C₂₋₆ alkenyl, C₂₋₆ alkynyl, C₁₋₆ haloalkyl, OH, CN, NO₂, and CO₂CH₃; wherein said C₁₋₆ alkyl and C₂₋₆ alkenyl are each optionally substituted with OH, CN, NO₂, or CO₂CH.

31. The method of claim 30, wherein the cancer is selected from bladder cancer, bone cancer, glioma, breast cancer, cervical cancer, colon cancer, colorectal cancer, endometrial cancer, epithelial cancer, esophageal cancer, Ewing's sarcoma, pancreatic cancer, gallbladder cancer, gastric cancer, gastrointestinal tumors, head and neck cancer, intestinal cancers, Kaposi's sarcoma, kidney cancer, laryngeal cancer, liver cancer, lung cancer, melanoma, prostate cancer, rectal cancer, renal clear cell carcinoma, skin cancer, stomach cancer, testicular cancer, thyroid cancer, and uterine cancer.

32. The method of claim 30, wherein the cancer is selected from lung cancer, colorectal cancer, and gastric cancer.

33. The method of claim 30, wherein R¹ is a peptide capable of selectively delivering R²L- across a cell membrane having an acidic or hypoxic mantle having a pH less than about 6.0.

34. The method of claim 30, wherein R¹ is a peptide comprising at least one of the following sequences:
(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG;
and
(SEQ ID No. 6; Pv6)
AAEQNPIYWWARYADWLFTTPLLLLDLALLVDADEGTCG;
wherein R¹ is attached to L through a cysteine residue of R¹.

35. The method of claim 30, wherein R¹ is a peptide comprising at least the following sequence:
(SEQ ID NO. 1; Pv1)
ADDQNPWRAYLDLLFPTDTLLLDLLWCG.

36. The method of claim 30, wherein R¹ is a peptide comprising at least the following sequence:
(SEQ ID NO. 2; Pv2)
AEQNPIYWARYADWLFTTPLLLLDLALLVDADECG.

37. The method of claim 30, wherein R¹ is a peptide comprising at least the following sequence:
(SEQ ID NO. 3; Pv3)
ADDQNPWRAYLDLLFPTDTLLLDLLWDADECG.

38. The method of claim 30, wherein R¹ is a peptide comprising at least the following sequence:
(SEQ ID NO. 6; Pv6)
AAEQNPIYWWARYADWLFTTPLLLLDLALLVDADEGTCG.

39. The method of claim 30, wherein R² is:

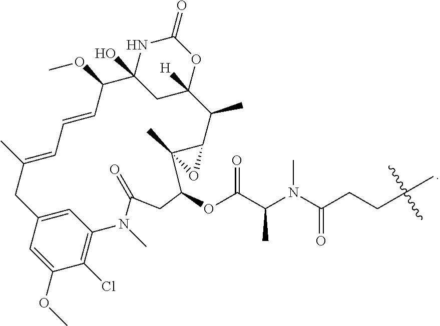

40. The method of claim 30, wherein R² is:

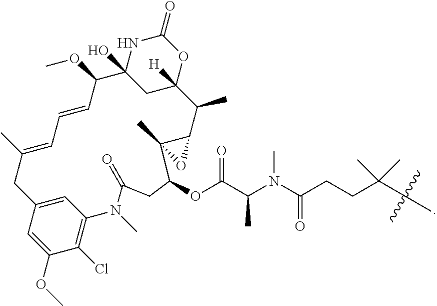

41. The method of claim 30, wherein R² is:

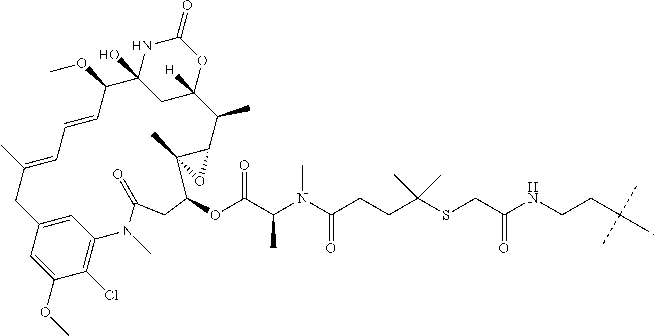

42. The method of claim 30, wherein R² is:

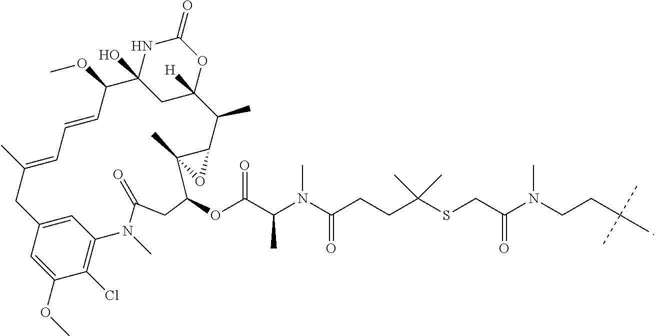

43. The method of claim 30, wherein L is:

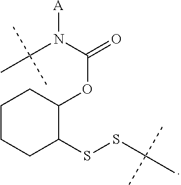

44. The method of claim 30, wherein R³, R⁴, R⁵, and R⁶ are each independently selected from H and C₁₋₄ alkyl.

45. The method of claim 30, wherein R³, R⁴, R⁵, and R⁶ are each H.

46. The method of claim 30, wherein A is H.

47. The method of claim 30, wherein A is CH₃.

48. The method of claim 30, wherein the compound is selected from: 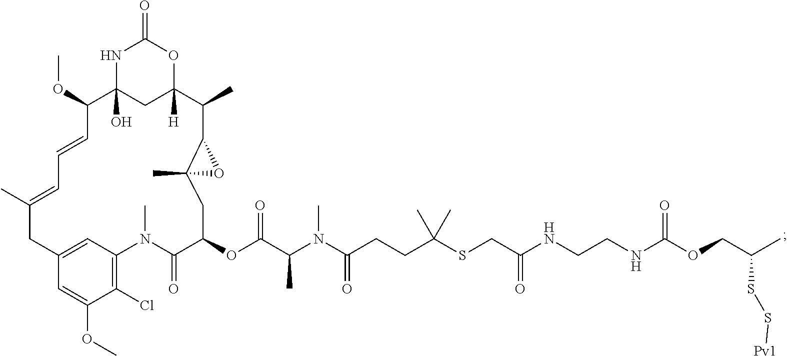 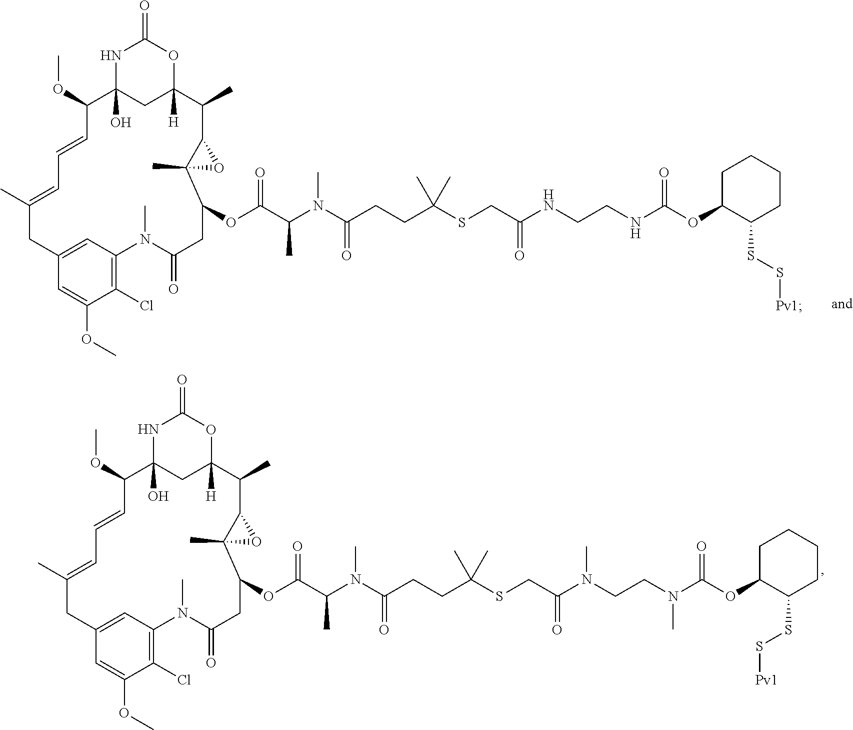
or a pharmaceutically acceptable salt of any of the aforementioned,
wherein Pv1 is a peptide comprising at least the following sequence:
(SEQ ID NO. 1)
ADDQNPWRAYLDLLFPTDTLLLDLLWCG

49. The method of claim 30, wherein the compound is: 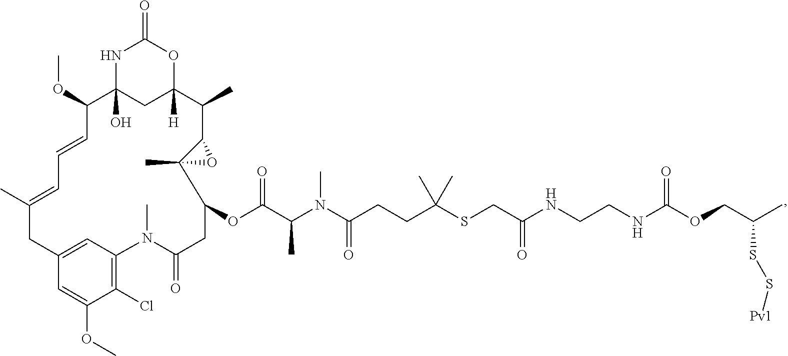
or a pharmaceutically acceptable salt thereof, wherein Pv1 is a peptide comprising at least the following sequence:
(SEQ ID NO. 1)
ADDQNPWRAYLDLLFPTDTLLLDLLWCG

50. The method of claim 30, wherein the compound is: 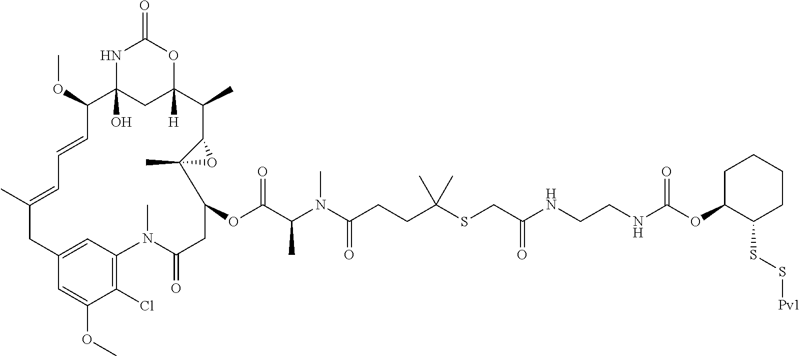
or a pharmaceutically acceptable salt thereof, wherein Pv1 is a peptide comprising at least the following sequence:
(SEQ ID NO. 1)
ADDQNPWRAYLDLLFPTDTLLLDLLWCG

51. The method of claim 30, wherein the compound is: 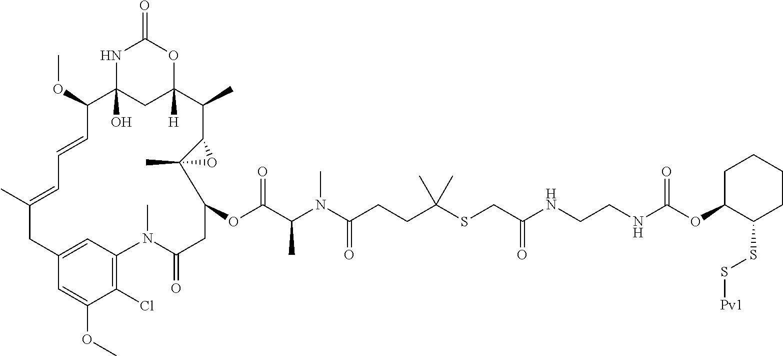
or a pharmaceutically acceptable salt thereof, wherein Pv1 is a peptide comprising at least the following sequence:
(SEQ ID NO. 1)
ADDQNPWRAYLDLLFPTDTLLLDLLWCG
